SEQUENCE LISTING (63 sequences)

SEQ ID NO: 1 - DNA, 1835 bp, Homo sapiens
caacagacacgctggagtttaacaaacagcaatactcttcgcgctcctgaaaagcaggtctggacgctctccgtggtgctgaaacgcctcgcagccgccgctgtccgtggtatctacgaccccctcgctccaatttcccctggggctctccctccgcgcccctgttccccgcctccctttaacatctggattattttttgcaatagcgctttctggttttgtaagtgccaatttgaaacatttttgcccccataactcgtggactacaaagcacaaggacctgaaaaatgtacagcttcaatactcttcgactctacctttgggagaccattgtattcttcagccttgctgcttctaaggaggctgaagctgctcgctccgcacccaagcctatgtcaccctcggatttcctggataagctaatggggagaacctccggatatgatgccaggatcaggcccaattttaaaggtcccccagtgaacgtgagctgcaacattttcatcaacagctttggttccattgctgagacaaccatggactatagggtcaacatcttcctgcggcagcaatggaacgacccccgcctggcctataatgaataccctgacgactctctggacctggacccatccatgctggactccatctggaaacctgacctgttctttgccaacgagaagggggcccacttccatgagatcaccacagacaacaaattgctaaggatctcccggaatgggaatgtcctctacagcatcagaatcaccctgacactggcctgccccatggacttgaagaatttccccatggatgtccagacatgtatcatgcaactggaaagctttggatatacgatgaatgacctcatctttgagtggcaggaacagggagccgtgcaggtagcagatggactaactctgccccagtttatcttgaaggaagagaaggacttgagatactgcaccaagcactacaacacaggtaaattcacctgcattgaggcccggttccacctggagcggcagatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtgaagggtctgaaaggttgggggaggctgggagaggggaacgtgggaatagcacaggaatctgagagactaaggaagagaaggggaacggagggagggggcacacttacacaactctctctgcaatatgtgcaatagcaaaatgcagtgatgcatgaattttaaaaaaaaaaaaaaa

SEQ ID NO: 2 - protein, 457 aa, Homo sapiens
MYSFNTLRLYLWETIVFFSLAASKEAEAARSAPKPMSPSDFLDKLMGRTSGYDARIRPNFKGPPVNVSCNIFINSFGSIAETTMDYRVNIFLRQQWNDPRLAYNEYPDDSLDLDPSMLDSIWKPDLFFANEKGAHFHEITTDNKLLRISRNGNVLYSIRITLTLACPMDLKNFPMDVQTCIMQLESFGYTMNDLIFEWQEQGAVQVADGLTLPQFILKEEKDLRYCTKHYNTGKFTCIEARFHLERQMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 3 - DNA, 3356 bp, Homo sapiens
tccttaaaggcgcgcgagccgagcggcgaggtgcctctgtggccgcaggcgcaggcccgggcgacagccgagacgtggagcgcgccggctcgctgcagctccgggactcaacatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaattcttctgggcattgccagtacctgcctccaggcatattcaagagttcctgctacatcgatgtacgctggtttccctttgatgtgcagcactgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggacgctctactatggcctcaacctgctgatcccctgtgtgctcatctccgccctcgccctgctggtgttcctgcttcctgcagattccggggagaagatttccctggggataacagtcttactctctcttaccgtcttcatgctgctcgtggctgagatcatgcccgcaacatccgattcggtaccattgatagcccagtacttcgccagcaccatgatcatcgtgggcctctcggtggtggtgacagtgatcgtgctgcagtaccaccaccacgaccccgacgggggcaagatgcccaagtggaccagagtcatccttctgaactggtgcgcgtggttcctgcgaatgaagaggcccggggaggacaaggtgcgcccggcctgccagcacaagcagcggcgctgcagcctggccagtgtggagatgagcgccgtggcgccgccgcccgccagcaacgggaacctgctgtacatcggcttccgcggcctggacggcgtgcactgtgtcccgacccccgactctggggtagtgtgtggccgcatggcctgctcccccacgcacgatgagcacctcctgcacggcgggcaaccccccgagggggacccggacttggccaagatcctggaggaggtccgctacattgccaaccgcttccgctgccaggacgaaagcgaggcggtctgcagcgagtggaagttcgccgcctgtgtggtggaccgcctgtgcctcatggccttctcggtcttcaccatcatctgcaccatcggcatcctgatgtcggctcccaacttcgtggaggccgtgtccaaagactttgcgtaaccacgcctggttctgtacatgtggaaaactcacagatgggcaaggcctttggcttggcgagatttgggggtgctaatccaggacagcattacacgccacaactccagtgttcccttctggctgtcagtcgtgttgcttacggtttctttgttactttaggtagtagaatctcagcactttgtttcatattctcagatgggctgatagatatccttggcacatccgtaccatcggtcagcagggccactgagtagtcattttgcccattagcccactgcctggaaagcccttcggagagctccccatggctcctcaccaccgagacagttggttttgcatgtctgcatgaaggtctacctgaaaattcaacatttgctttttgcttgtgtacaaacccagattgaagctaaaataaaccagactcactaaatcctttccaataattgactggtggaaggaaaacaaaaaacaaaaactaaaaacctcttagcttttctgcaattcaactttttatttttatttttatttctatcaaagacggtagagagaaacagcttgatgctgtttctacattaaaaaaaaaaaaaaaagacagactgttggtcttactaaggatgtttttaccagcctgcctgacttctgcaaacctaccctgtcaaggagatcaaagggacgcaggtttctgtttattctgaacaagggccaggccccgcggagtgtctttggtggatcccagataactcctaggtgctgctctcagacactgaggagttgagcaaatctgttctattctgcagaacccacaggacaaataagagttctactagaattaacagcccaaaagaatagctacagctaagtgaagccacttacgtgggctttaaaaaaataatgtgttagctgattcacatgcactggagttaattagtcttagaaatgtgtgcatccatacaaatgcacaacataaagtgaacatattcctaggccctttctgcctgtgtcagggccaggaagtagaggctgggaactcttctggtccccagtatggcaggcgccagggaggggatggtgtggcccatcccttctctggatacctggccagtggcaggcagcagggaggagctggccgaccctcagtgactgacaagccagcaattctgagttctggcctttgggagtctgcctgctccaagccagtccaccccagctgcagccccaaaagctggctcaaagtccttgggtggattcactggagatgggcaacttaaaacaagagaaactttaatttttaaacctaagtgatgatacagctcttcccttagattatcgcccaggctggagtgcagtggcatgatctcagctcactgcaagctccacctcctgggttcatgccattctcctgcctcagcctccccccgagtaactgggaatacaggcgcccgccaccatgcctggctaattttttgtatttttagtagagatagggtttcatcatgttagccaggatggtctcattcttattctttaatgagatcagagggtaattcaccaagaaagacctctcctgttccattgtgtcatccaacaactgctcagagctcaaaattatagaaggcttctgagcccctagagatttttaatttgcttctaatccctgaggtgggaacatcatgagggaagatttgattttcagagttaaataaattgtatgtgcttttccagccaaaaaaaaaaa

SEQ ID NO: 4 - protein, 502 aa, Homo sapiens
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRTLYYGLNLLIPCVLISALALLVFLLPADSGEKISLGITVLLSLTVFMLLVAEIMPATSDSVPLIAQYFASTMIIVGLSVVVTVIVLQYHHHDPDGGKMPKWTRVILLNWCAWFLRMKRPGEDKVRPACQHKQRRCSLASVEMSAVAPPPASNGNLLYIGFRGLDGVHCVPTPDSGVVCGRMACSPTHDEHLLHGGQPPEGDPDLAKILEEVRYIANRFRCQDESEAVCSEWKFAACVVDRLCLMAFSVFTIICTIGILMSAPNFVEAVSKDFA

SEQ ID NO: 5 - DNA, 2343 bp, Homo sapiens
ctctcagctgtcccctcccctttcctcccgcctgaaacatgatccagctgaaggactgattgcaggaaaacttggcagctccccaaccttggtggcccagggagtgtgaggctgcagcctcagaaggtgtgagcagtggccacgagaggcaggctggctgggacatgaggttggcagagggcaggcaagctggcccttggtgggcctcgtcctgagcactcggaggcactcctatgcttggaaagctcgctatgctgctgtgggtccagcaggcgctgctcgccttgctcctccccacactcctggcacagggagaagccaggaggagccgaaacaccaccaggcccgctctgctgaggctgtcggattaccttttgaccaactacaggaagggtgtgcgccccgtgagggactggaggaagccaaccaccgtatccattgacgtcattgtctatgccatcctcaacgtggatgagaagaatcaggtgctgaccacctacatctggtaccggcagtactggactgatgagtttctccagtggaaccctgaggactttgacaacatcaccaagttgtccatccccacggacagcatctgggtcccggacattctcatcaatgagttcgtggatgtggggaagtctccaaatatcccgtacgtgtatattcggcatcaaggcgaagttcagaactacaagccccttcaggtggtgactgcctgtagcctcgacatctacaacttccccttcgatgtccagaactgctcgctgaccttcaccagttggctgcacaccatccaggacatcaacatctctttgtggcgcttgccagaaaaggtgaaatccgacaggagtgtcttcatgaaccagggagagtgggagttgctgggggtgctgccctactttcgggagttcagcatggaaagcagtaactactatgcagaaatgaagttctatgtggtcatccgccggcggcccctcttctatgtggtcagcctgctactgcccagcatcttcctcatggtcatggacatcgtgggcttctacctgccccccaacagtggcgagagggtctctttcaagattacactcctcctgggctactcggtcttcctgatcatcgtttctgacacgctgccggccactgccatcggcactcctctcattggtaaggcccctcctggcagcagagctcagtctggtgagaaacccgccccctcccacctcctgcatgtgtctcttgcctctgccctgggctgcacaggtgtctactttgtggtgtgcatggctctgctggtgataagtttggccgagaccatcttcattgtgcggctggtgcacaagcaagacctgcagcagcccgtgcctgcttggctgcgtcacctggttctggagagaatcgcctggctactttgcctgagggagcagtcaacttcccagaggcccccagccacctcccaagccaccaagactgatgactgctcagccatgggaaaccactgcagccacatgggaggaccccaggacttcgagaagagcccgagggacagatgtagccctcccccaccacctcgggaggcctcgctggcggtgtgtgggctgctgcaggagctgtcctccatccggcaattcctggaaaagcgggatgagatccgagaggtggcccgagactggctgcgcgtgggctccgtgctggacaagctgctattccacatttacctgctagcggtgctggcctacagcatcaccctggttatgctctggtccatctggcagtacgcttgagtgggtacagcccagtggaggagggggtacagtcctggttaggtggggacagaggatttctgcttaggcccctcaggacccagggaatgccagggacattttcaagacacagacaaagtcccgtgccctgtttccaatgccaattcatctcagcaatcacaagccaaggtctgaacccttccaccaaaaactgggtgttcaaggcccttacacccttgtcccacccccagcagctcaccatggctttaaaacatgctgtcttagatcaggagaaactcgggcactccctaagtccactctagttgtggacttttccccattgaccctcacctgaataagggactttggaattctgcttctctttcacaactttgcttttaggttgaaggcaaaaccaactctctactacacaggcctgataactctgtacgaggcttctctaacccctagtgtcttttttttcttcacctcacttgtggcagcttccctgaacactcatcccccatcagatgatgggagtgggaagaataaaatgcagtgaaaccctaaaaaaaaaaaa

SEQ ID NO: 6 - protein, 516 aa, Homo sapiens
MLGKLAMLLWVQQALLALLLPTLLAQGEARRSRNTTRPALLRLSDYLLTNYRKGVRPVRDWRKPTTVSIDVIVYAILNVDEKNQVLTTYIWYRQYWTDEFLQWNPEDFDNITKLSIPTDSIWVPDILINEFVDVGKSPNIPYVYIRHQGEVQNYKPLQVVTACSLDIYNFPFDVQNCSLTFTSWLHTIQDINISLWRLPEKVKSDRSVFMNQGEWELLGVLPYFREFSMESSNYYAEMKFYVVIRRRPLFYVVSLLLPSIFLMVMDIVGFYLPPNSGERVSFKITLLLGYSVFLIIVSDTLPATAIGTPLIGKAPPGSRAQSGEKPAPSHLLHVSLASALGCTGVYFVVCMALLVISLAETIFIVRLVHKQDLQQPVPAWLRHLVLERIAWLLCLREQSTSQRPPATSQATKTDDCSAMGNHCSHMGGPQDFEKSPRDRCSPPPPPREASLAVCGLLQELSSIRQFLEKRDEIREVARDWLRVGSVLDKLLFHIYLLAVLAYSITLVMLWSIWQYA

SEQ ID NO: 7 - DNA, 5723 bp, Homo sapiens
gctccgggccagcgcggcggcggcggcggcggcggcagcagcaggagcagccccggctgcgggtcgcgacggcggcggggcgccccctcccccgtgccggggcgcggcggagggatgtggggccttgcgggaggaaggcttttcggcatcttctcggccccggtgctggtggctgtggtgtgctgcgcccagagtgtgaacgatcccgggaacatgtcctttgtgaaggagacggtggacaagctgttgaaaggctacgacattcgcctaagacccgacttcgggggtcccccggtctgcgtggggatgaacatcgacatcgccagcatcgacatggtttccgaagtcaacatggattataccttaaccatgtattttcaacaatattggagagataaaaggctcgcctattctgggatccctctcaacctcacgcttgacaatcgagtggctgaccagctatgggtgcccgacacatatttcttaaatgacaaaaagtcatttgtgcatggagtgacagtgaaaaaccgcatgatccgtcttcaccctgatgggacagtgctgtatgggctcagaatcaccacgacagcagcatgcatgatggacctcaggagataccccctggacgagcagaactgcactctggaaattgaaagctatggctacaccacggatgacattgagttttactggcgaggcggggacaaggctgttaccggagtggaaaggattgagctcccgcagttctccatcgtggagcaccgtctggtctcgaggaatgttgtcttcgccacaggtgcctatcctcgactgtcactgagctttcggttgaagaggaacattggatacttcattcttcagacttatatgccctctatactgataacgattctgtcgtgggtgtccttctggatcaattatgatgcatctgctgctagagttgccctcgggatcacaactgtgctgacaatgacaaccatcaacacccaccttcgggagaccttgcccaaaatcccctatgtcaaagccattgacatgtaccttatgggctgcttcgtctttgtgttcctggcccttctggagtatgcctttgtcaactacattttctttggaagaggccctcaaaggcagaagaagcttgcagaaaagacagccaaggcaaagaatgaccgttcaaagagcgaaagcaaccgggtggatgctcatggaaatattctgttgacatcgctggaagttcacaatgaaatgaatgaggtctcaggcggcattggcgataccaggaattcagcaatatcctttgacaactcaggaatccagtacaggaaacagagcatgcctcgagaagggcatgggcgattcctgggggacagaagcctcccgcacaagaagacccatctacggaggaggtcttcacagctcaaaattaaaatacctgatctaaccgatgtgaatgccatagacagatggtccaggatcgtgtttccattcactttttctcttttcaacttagtttactggctgtactatgttaactgagtgactgtacttgatttttcaaagacttcatttaacactgagtgaaatattactctgcctgtcaagtttttatacctgtacacacacagacacacaagcagacacacacatatatacatacgcaattgtatatatatgtgaactttctcagcatatatataaaatacacgtgtatatgaggatgtatgtgtatatgtttatacacacaggagtcagtgcccatgtgtatggaagacaaatacacatacatatatacattttgcagctatggacaatttaccacaggatgcatattaaagaaagtcatagtttttttcttttttaattgaaagggacaagtatcatctaaatattatgccttgagaatgagggcgtgaaacacaatatcatccccaaatgtgtcttgtattatcataagttagatgttttagtttaaaaatcagaaagacattcttagttaatctttgaaaactcatacagtggtattgctagtttaaaatgagtcacttacttcatatcctctcgttcagtttagtaagcaaaggcttcttggcttctctggtgatggggtttgttttcatcgggcatacgttttctgcaatggtttagtggctggggtgagccactggcagtgtgcttacctgttgtctgaaacatagatagatcccacgttgatgtctgaacgaccgtcttttgaaaactcatcgggagtgaatggcatctcgttgtaagtactctaatatacagtgtgtagtttgtttctgttgttcacttggagtggatccagcttcactgtcatgtgcgaacacagtgacacgtttggccagtgacatttcaatcactgaaaatgtgctctacatctcgtatggatttctaggcctgatatccaacagaaagcatagacgtctcaggttattcgttactctaaggtaaaaccatctaggatgattttcccccttgcagttatgttatcattcttataacattgtatgttaatagaaaatatatttgcataatatgcatatatatgtatatttaccaagattttgtttcttacgcttgctatcatggcagcatgcgatgtcatattttcctttatgtgatgtaactactttctgttatctagaaattaagattgaagctaaaacacttctactgttcaatttcagaaactaagaatcatcctcatgcctttatttctgtatctgacatatttcataagcacatccaactactcctagactgactaggattctgcaggaacatgacccgtacacaccacgcgtcacccaacgacccatgaccgttctctgaggcaaaggagggcaacctgacagcaaacacagtcactgttggttccttctgatccacagcctcatcagtatttggactttttaaagctcgtagaaacaagacaaggtgcaccggtttcatagacgcaaccttaacttactatttagatgagatctttctaaagaaaaaaaaaagagatgatatattttttgtaaacaatatttctatcacaggcatccataaactgaaatgactacagttgtgcaaacaggtgtcacagtgaagttgagcatttggagaaaaaaataaaaagcaaaatttgcaggaagaactgctaaattaatactttatcccaaaatgccacgtatgcctcaccctctctgttctatccaaaaccaaggaccagagtgctccaatggtaggccccagttgtctcgatgtaggtagaggcaccaccctccccgaggatgcgtgtggtgtggactatccccatgagctgaccactacttgatttttctttggtggccgtaacaacctttaatttgtgggcatctgcaacagttcaaaacccaccatcagataaaacataatccacaaaatctcacgctagaggcaattacctacttttagacccttttccctctttcattcctatctttttcctcctaatcgctgctctctggttttattttcatctggagactagccagggatttctttggctttggcttttctctgaccattttttccatgggtaacaatgggggatcctaaaagttaaacagattgggaaaaaccttgttaagtggccttatcattattatcatgttaaagaaaaaaaatacatttgcaaagaccttatccctttcaatacttcaggtatctttttctgtatccagtaattaaaggtgtgaggtccacatgcagaagagggaccccaaaatgtaaatggatgtggacaaaaacagtcaatggtctatttaagtgtataatttatactattcagaactgtgacattaattctttctgggagaaaagtgatttaaaacttttttgatgcatagttgaggtacccaaatatcaaaggcagagaccccatggggctccaaagagctgcagtctccttcccaaggttttctggatataaatttgcatggtatagtcataatagcttttgagctttttatatgcatttggcaccaagactgggatccacaactttgtagacactgcgatgaagttaacatattagctatactataaatagtgtttgtaactacacacacacacacacacacacacacaaatacatacatatattctgtaaaaacaaaaaaaaactttttaagatccttgggtatgtgtttgctttactccatttcagaagaaaattacttttcttctaacaaaattattttatagcttctccatttttaaaacttgtgagagcattgagaagagaactctgactgtgttaacagaagagagttgaattggagtctctgttgtgttaaaatgacctctcgttactccacagtagttatttgagccagtgactgagtcgcgttgaggaattctgaacccggacctctgacgttgttgggaggtggctgttacccacacccagacctcttgagtaaggacagaaacgttatcattggggatataatgagatttctttcttaacaattgaaagtaataaagagttaattttctcagtagtcctgtctttccaaaatgcgccacaggggctcaagatctacagaagaatcttgttatgacagtttgttattacccatattcagatatccttgagataatggaagagccctgctacataactccctacagagaaagactgatagacttgagtagtccttaaaacaagtgttattcctgttcaccaaccccgggactgtggaggggtttcactgtctataccatgcgacatccatttcccctgaatctcaaacgaactagaaagtatgtcatgataatatttccattagatttggaagctacctgtacatctgcaatattgtgtttttaacgccagcacccagaactttgacatgttcagtcactccctgaaaggcacttctctcttgtccaaacacagcgttgacatttttactgaggagatcatctcaaaggtgatgccaaacgagtctggggctggttttaaggggacaggcacattgcagttgtaggtgttcattttcagcatctagtagataatccattggtgtttgtcccaccattggtgtattctaacaagaatgtgtccaactttgaatctcctggcttgaaagtataaaccatcacttaattcttattttaactctccacctgaaaaccagttcatatttctggccattttatgtaagcaaaactgaacaattggtgaaacattttgttactcttggaattgactctggctgtcagtgtgagccattagagtaacatcgaatcttggggcaaagaactgcccaggtgaattaaatttttccaggacactagctagtgtgccttggattgattacctcttctactgcattgaaaggcgccatgttttcctgaaatactaaattcccaacacctgggtaaacaatgaccttccagagagtggctcccgtatgcctctccctaggaccaaccccatgaacatgttttgtcacgtttgtctcatgtttctacttcacaagtcagtgagtgtgtttaaggtaagtacaggattattctagtaggaataggcgattgctgtcataatcaattctcatgttgatttcattttattgtaaagataaatttaaacccagttttgcttaagcacattgatgtaattttttggtattatttgacatgaaaaaacagcaaaattgagtgatagatac

SEQ ID NO: 8 - protein, 473 aa, Homo sapiens [sequence omitted]

SEQ ID NO: 9 - DNA, 3174 bp, Homo sapiens
taataatggccgtaagcttaaaatagatccagggaggagctcattaacgtgaacatagaaagcagttccgcacctctggccttactcctcttggaaattgctttggtccatttttacttccttttattcgacgcaccagaaaataagacttttaccaacatttttactgcatttgacgatgaactaatttagaccggctaaaataattgttccactgggacacaggaattcaacctcagttcagaaaatccctgacatctgacgtaggaggatttataggtttagtggaaattgctttctcctgctctccagattgcatcctgtgggttgattttttttttgcatgagtaaacatccttctaataatgaacagaccaataatgtcttaagagagaaaaagaacaatcttttcctttttgctgtttctggagagagctgtttgaatttggaaacccatgttggctgtcccaaatatgagatttggcatctttcttttgtggtggggatgggttttggccactgaaagcagaatgcactggcccggaagagaagtccacgagatgtctaagaaaggcaggccccaaagacaaagacgagaagtacatgaagatgcccacaagcaagtcagcccaattctgagacgaagtcctgacatcaccaaatcgcctctgacaaagtcagaacagcttctgaggatagatgaccatgatttcagcatgaggcctggctttggaggccctgccattcctgttggtgtggatgtgcaggtggagagtttggatagcatctcagaggttgacatggactttacgatgaccctctacctgaggcactactggaaggacgagaggctgtcttttccaagcaccaacaacctcagcatgacgtttgacggccggctggtcaagaagatctgggtccctgacatgtttttcgtgcactccaaacgctccttcatccacgacaccaccacagacaacgtcatgttgcgggtccagcctgatgggaaagtgctctatagtctcagggttacagtaactgcaatgtgcaacatggacttcagccgatttcccttggacacacaaacgtgctctcttgaaattgaaagctatgcctatacagaagatgacctcatgctgtactggaaaaagggcaatgactccttaaagacagatgaacggatctcactctcccagttcctcattcaggaattccacaccaccaccaaactggctttctacagcagcacaggctggtacaaccgtctctacattaatttcacgttgcgtcgccacatcttcttcttcttgctccaaacttatttccccgctaccctgatggtcatgctgtcctgggtgtccttctggatcgaccgcagagccgtgcctgccagagtccccttaggtatcacaacggtgctgaccatgtccaccatcatcacgggcgtgaatgcctccatgccgcgcgtctcctacatcaaggccgtggacatctacctctgggtcagctttgtgttcgtgttcctctcggtgctggagtatgcggccgtcaactacctgaccactgtgcaggagaggaaggaacagaagctgcgggagaagcttccctgcaccagcggattacctccgccccgcactgcgatgctggacggcaactacagtgatggggaggtgaatgacctggacaactacatgccagagaatggagagaagcccgacaggatgatggtgcagctgaccctggcctcagagaggagctccccacagaggaaaagtcagagaagcagctatgtgagcatgagaatcgacacccacgccattgataaatactccaggatcatctttccagcagcatacattttattcaatttaatatactggtctattttctcctagatgcttgtaattctacaaatttcacatttccatggcatgcactacagaaataactgtataatgaaaaagtatttaaggatatggttaaaaaaaaatcccaggacccacccatgttttcactatcccttctgcagctttccaaagctacattgacgagacacttactggtttaatttgcacttattaaccatctattgaatacacagcattatattaggtgctgcaggaaatacgacactgtagcgactgatgttagttgttacccagatcccctggaaaagcacactaccagtgttgtgggcacatttagttccacccgttagacccttgatgctattcacatgaataatttattttcctcaagtgtcattacattgttcaggctacgtgaacttggaagcacctacaggccatttgcatgaaattcacatgcacctaaatcctcactttgacagaaactcatgcttcagtttataacctattacctattttgtatgcgactccacctccgcatgtttattttaataaaaggcaatgataacattcacattatttttctttatatgctgtggttcacaggctttaccccttcacaagaaaagctctttagattggcgcaattgcttctgattttggtgaaattttccctggtagggaaactttgaagataagagtacacacatgcattttgtctgttgtgtcatagaggtaactaggctagaaaatttgtgtttaaatgttccctattttatataatcaccacttcatgtttcttcttcttggagcatgtccttgttcaaagagaagtgctttctcagtgatgtgatatcttcactgaggaacttgggtagagaatgatttcttctgcataaacacttcaaggaaatacataatttgggactacttgtaactcattagaatgagaaatactcacatggtttcttaagagaaaaagaacatcggaaagcaaaataaatgggaagatatcactggacatctgcatttatactcgaaataccagcattttctatggaccagaaaactgccatcacctagaccacacagcccagataccaggcagacggatggcccaatggcaactgatgtcagggcatggggtaaaggagagggttctaatctggtgtatcacttaaaaacagttatttatattatatatctgctatatagatcaacctccaccaaacttacccaaacagcatttgttttatttgaaactcactttaataaagtgaattatatacacaaaaaaaaaaaaaaa

SEQ ID NO: 10 - protein, 479 aa, Homo sapiens
MLAVPNMRFGIFLLWWGWVLATESRMHWPGREVHEMSKKGRPQRQRREVHEDAHKQVSPILRRSPDITKSPLTKSEQLLRIDDHDFSMRPGFGGPAIPVGVDVQVESLDSISEVDMDFTMTLYLRHYWKDERLSFPSTNNLSMTFDGRLVKKIWVPDMFFVHSKRSFIHDTTTDNVMLRVQPDGKVLYSLRVTVTAMCNMDFSRFPLDTQTCSLEIESYAYTEDDLMLYWKKGNDSLKTDERISLSQFLIQEFHTTTKLAFYSSTGWYNRLYINFTLRRHIFFFLLQTYFPATLMVMLSWVSFWIDRRAVPARVPLGITTVLTMSTIITGVNASMPRVSYIKAVDIYLWVSFVFVFLSVLEYAAVNYLTTVQERKEQKLREKLPCTSGLPPPRTAMLDGNYSDGEVNDLDNYMPENGEKPDRMMVQLTLASERSSPQRKSQRSSYVSMRIDTHAIDKYSRIIFPAAYILFNLIYWSIFS

SEQ ID NO: 11 - DNA, 4620 bp, Homo sapiens
atgccttattttacaagactcattttgttcttgttttgcttgatggttctcgtggagagcagaaaacccaagaggaagcgatggacagggcaggtggaaatgcccaagccaagtcacttatataagaagaaccttgatgtgaccaagatccggaagggaaagcctcagcagcttctcagagtggacgagcacgacttcagcatgagacccgccttcggaggccctgccatcccggtgggcgtggacgtacaggtggagagcctggacagcatctccgaggtggacatggacttcactatgaccctgtacctgcggcattactggaaggatgagaggctagctttctccagcgccagcaacaagagcatgaccttcgatggccggctggtgaagaagatctgggtccctgatgtcttctttgttcactccaaaagatcgttcactcatgacaccaccactgacaacatcatgctgagggtgttcccagatggacacgtgctgtacagcatgaggattacggtcactgccatgtgcaacatggacttcagccactttcccctggactcccagacctgttctttggagctggagagctatgcctatacagatgaagatctaatgctgtactggaagaatggggatgaatccctaaaaacagatgagaagatctccttgtctcagtttctgattcagaaatttcacacaacttccaggctggccttctacagcagcactggctggtacaaccgtctgtacattaacttcacgttgcgtcgccacatcttcttcttcttgctccaaacatatttccctgccactctgatggtcatgctgtcctgggtgtccttctggatcgaccgcagagctgtgcctgccagagtttcactgggtatcacgacggtgctgaccatgaccaccatcatcacgggcgtgaatgcctccatgccgcgcgtctcctacgtcaaggccgtggacatctacctctgggtcagctttgtgttcgtgttcctctcggtgctggagtatgcggctgtcaactacctgaccaccgtgcaggagcgcaaggaacggaagctgcgggagaagttcccgtgcatgtgtggaatgcttcattcaaaaaccatgatgctggatggaagctacagtgagtctgaggccaacagcctggctgggtaccccagaagccatatcctgacagaagaagaaaggcaagacaaaatagtggtccacctgggcctgagtggtgaagccaacgctgccagaaagaaggggcttctgaagggccagacgggttttcgtatcttccagaatacccatgccattgacaaatactctaggttgatattccctgcctcctacatatttttcaacttaatttattggtcagtgttttcctaggggctccaaggctgttcctagaagagggcatagacatcgagggggcctggccagtcattgacagacggacttgttgaccacacgcccctcaccaaacaatgcagcagctactggaccaccctgagcagcactcatctctcagagaagcccaggagccttccagctgccctgaccccagaccccgtggggctgctccatgttcatgctgtctcgcgtcacacttcacatctctctgggaccttctgttcttgtgtgtgaactaattcacaagaactcccctcctataaacaaggattcaaatgcctcctagacattcttagaccctcagattgcctaggagtcagttgtggagggaaaaggaaaaactgaagtacaagcttaggggcattctggataggagataggaaattaagaacaaaaaacaccctgtaaacccattgaacttgattaagtgcctacctagaagggaaaagagctgagatcccagacaggaaatgatttggcctgtgggagtatggcaacccacaggattctgcaatattagaggagattattcatttatccatccattcaaccattcattaaatattgaggtcccactctgtgccaggtacggtagaaatgagaaaatagtctctttccttagagatcttctctaagttgtgctcattctacatatggcatgatgtacttctgctctttcccctccttcaaccccgcttaccccacagacccattctgtttgctgttgcttttactcttaggcatataaggcagggtccaggagaggccaggtccagaactttcaactatcctcctccagtgaagttacacagatagcactaattttgcccagcaatgacatgtagcaatgtgcatggagtattgccagccagagaaacttacccaggctcaccacggtgtacagtttttttattggggtcggtcatggatttatagctgattgcccacatggctgactttagtcttttgcccctccagaggttaagctggtaccttgaggtccagggcacctatcataaatcacattgttggcatagactatctggtgtggcccaaggccccagtagacaaagacccttctatctcacaggacattccaagggcatagaggttgcttcctagtaaaggttagtcctttactacacagcaactcttgtcaattccctattgttaatgttaattgcggtgttactattatatattcaggcaataaacattttaaagtatctctgcactaggtacattggggtccaaattaagaataagagtagtcgaagctctcagagaaggaactagtaaaggaaataccacacaaatatctataatataagattaagagtgtttagtgcctcagtaatagttcagtgtgagatgggaattccaagggaggaaaaacagtcccaccagggagacagacctcatggagaaagcagccctgagatgccccttgaaggtcaggattttaagaagaagaaatggagcagaatgcatcctagatctgggctgtctcatatggtagccactagccacatgtctatttaaatttaaattagtaaaaattaaaaatattagccaggtgtggtggtgtgtgcctggggttctagctactcaggaggctgaggcgggaggattgcttgagcccaggaattccaggctgccgtgagctgtgatcaagccactgcagtccagcctgagtgacagagcgagaccctatctctaaaataataattattattatttaaattagttgaagttaaataaaattgaaaactcagttccttggttacaccatccacactcaagtgggcttagtaagcacagttgctagtagctactgcactgggctgggcagcctaaagaccaggaagctctaccagccaagagaccagcttgagcggacgcatggggagaatgggaaggtttgccattgcagagaacagggtttgtgggcaagtcattcattttggcaggatcctaggctgtgataagaggaggtgagcgactagctttttccacaacactagggatcaaggcaggtccccaccatgccggtgcttatgaggacagcttccccactatggagttgctggacccaagactactagatttcccagtctcacagaccaatgaagaacaaaaatgcataaatatgggtaattgggtggtacagggcatctggacttgtttagaaacctttttttcttttctgagacagagtttcactcttgttgctcaggctggagtgcaatggcttgatcttggctcaccacaacctctgcctcccgggttcaagcaattctcctgcctcagccctggagtagctgggattacaggcatgcgccaccatgcctggctaacttttgtattttttagtagagatggggtttctccatgttagtcagcctggtcttgaactcctgacctcaggtgatccgcctgcctcagcctcccaaagtgctgtgattacaggcgtgagccaccacacccggccagaaaccttttatctatagagactctcccaggccacagtgctgctcgaagaatgataattctattgtcattaaatatatcatattgctttgcttgtaagtcttatttaattccaacccttctctgcttctgactgcattgttttcatgcattatggctcatataacatagttgcctgttcacaaattaaaacaaatatttatttttgctagcttctatgttaaactggagttaattctatagcagacatatttagaatcataaacagtgttttatcatgcagggccatgatacaatgacaccattgtatttagcattgccttattacagtatactagtctgacactacttgagaaaagtaggaaatttaaagaggtaaagtggagacaacattggtaggagtagaaaacaggaactaaggaaatagtgagaacatgggatgattcaccctggaggcaggaagacagagaagcaaacacaattgttatcaggaatgcatgaatgatactttacttcactgaatctaagacatggtgaaatgtaagatgcacccatattttatgtactgagaaaaaatactaacaatcaaattctggaatgccaatgattataatatgaatcctgatttcaccaatatgaaactgtgaaaaaatgttaatgttacaataaaagaaatgggttttatacaaaaaaaaaaaaaaaaaaaaa

SEQ ID NO: 12 - protein, 465 aa, Homo sapiens
MPYFTRLILFLFCLMVLVESRKPKRKRWTGQVEMPKPSHLYKKNLDVTKIRKGKPQQLLRVDEHDFSMRPAFGGPAIPVGVDVQVESLDSISEVDMDFTMTLYLRHYWKDERLAFSSASNKSMTFDGRLVKKIWVPDVFFVHSKRSFTHDTTTDNIMLRVFPDGHVLYSMRITVTAMCNMDFSHFPLDSQTCSLELESYAYTDEDLMLYWKNGDESLKTDEKISLSQFLIQKFHTTSRLAFYSSTGWYNRLYINFTLRRHIFFFLLQTYFPATLMVMLSWVSFWIDRRAVPARVSLGITTVLTMTTIITGVNASMPRVSYVKAVDIYLWVSFVFVFLSVLEYAAVNYLTTVQERKERKLREKFPCMCGMLHSKTMMLDGSYSESEANSLAGYPRSHILTEEERQDKIVVHLGLSGEANAARKKGLLKGQTGFRIFQNTHAIDKYSRLIFPASYIFFNLIYWSVFS

SEQ ID NO: 13 - DNA, 1404 bp, Homo sapiens
atggtcctggctttccagttagtctccttcacctacatctggatcatattgaaaccaaatgtttgtgctgcttctaacatcaagatgacacaccagcggtgctcctcttcaatgaaacaaacctgcaaacaagaaactagaatgaagaaagatgacagtaccaaagcgcggcctcagaaatatgagcaacttctccatatagaggacaacgatttcgcaatgagacctggatttggagggtctccagtgccagtaggtatagatgtccatgttgaaagcattgacagcatttcagagactaacatggactttacaatgactttttatctcaggcattactggaaagacgagaggctctcctttcctagcacagcaaacaaaagcatgacatttgatcatagattgaccagaaagatctgggtgcctgatatcttttttgtccactctaaaagatccttcatccatgatacaactatggagaatatcatgctgcgcgtacaccctgatggaaacgtcctcctaagtctcaggataacggtttcggccatgtgctttatggatttcagcaggtttcctcttgacactcaaaattgttctcttgaactggaaagctatgcctacaatgaggatgacctaatgctatactggaaacacggaaacaagtccttaaatactgaagaacatatgtccctttctcagttcttcattgaagacttcagtgcatctagtggattagctttctatagcagcacaggttggtacaataggcttttcatcaactttgtgctaaggaggcatgttttcttctttgtgctgcaaacctatttcccagccatattgatggtgatgctttcatgggtttcattttggattgaccgaagagctgttcctgcaagagtttccctgggaatcaccacagtgctgaccatgtccacaatcatcactgctgtgagcgcctccatgccccaggtgtcctacctcaaggctgtggatgtgtacctgtgggtcagctccctctttgtgttcctgtcagtcattgagtatgcagctgtgaactacctcaccacagtggaagagcggaaacaattcaagaagacaggaaagatttctaggatgtacaatattgatgcagttcaagctatggcctttgatggttgttaccatgacagcgagattgacatggaccagacttccctctctctaaactcagaagacttcatgagaagaaaatcgatatgcagccccagcaccgattcatctcggataaagagaagaaaatccctaggaggacatgttggtagaatcattctggaaaacaaccatgtcattgacacctattctaggattttattccccattgtgtatattttatttaatttgttttactggggtgtatatgtatga

SEQ ID NO: 14 - protein, 467 aa, Homo sapiens
MVLAFQLVSFTYIWIILKPNVCAASNIKMTHQRCSSSMKQTCKQETRMKKDDSTKARPQKYEQLLHIEDNDFAMRPGFGGSPVPVGIDVHVESIDSISETNMDFTMTFYLRHYWKDERLSFPSTANKSMTFDHRLTRKIWVPDIFFVHSKRSFIHDTTMENIMLRVHPDGNVLLSLRITVSAMCFMDFSRFPLDTQNCSLELESYAYNEDDLMLYWKHGNKSLNTEEHMSLSQFFIEDFSASSGLAFYSSTGWYNRLFINFVLRRHVFFFVLQTYFPAILMVMLSWVSFWIDRRAVPARVSLGITTVLTMSTIITAVSASMPQVSYLKAVDVYLWVSSLFVFLSVIEYAAVNYLTTVEERKQFKKTGKISRMYNIDAVQAMAFDGCYHDSEIDMDQTSLSLNSEDFMRRKSICSPSTDSSRIKRRKSLGGHVGRIILENNHVIDTYSRILFPIVYILFNLFYWGVYV

SEQ ID NO: 15 - DNA, 1320 bp, Artificial Sequence
  synthetic sequence of chimeric LGIC
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgctacatcgatgtacgctggtttccctttgatgtgcagcactgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtga

SEQ ID NO: 16 - protein, 439 aa, Artificial Sequence
  synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 17 - protein, 439 aa, Artificial Sequence
  synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRQMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 18 - protein, 439 aa, Artificial Sequence
  synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVHLERQMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 19 - protein, 439 aa, Artificial Sequence
  synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRTLYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 20 - DNA, 1329 bp, Artificial Sequence
  synthetic sequence of chimeric LGIC
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgctacatcgatgtacgctggtttccctttgatgtgcagcactgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctgagatcatgcccgcaacatccgattcggtatcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtga

SEQ ID NO: 21 - protein, 442 aa, Artificial Sequence
  synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSEIMPATSDSVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 22 - DNA, 1332 bp, Artificial Sequence
  synthetic sequence of chimeric LGIC
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgctacatcgatgtacgctggtttccctttgatgtgcagcactgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctgagatcatgcccgcaacatccgattcggtaccattgatagcccaggccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtga

SEQ ID NO: 23 - protein, 443 aa, Artificial Sequence
  synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSEIMPATSDSVPLIAQAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 24 - DNA, 1323 bp, Artificial Sequence
  synthetic sequence of chimeric LGIC
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgctacatcgatgtacgctggtttccctttgatgtgcagcactgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtccgattcggtaccattgattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtga

SEQ ID NO: 25 - protein, 440 aa, Artificial Sequence
  synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSRASLPKVSDSVPLIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 26 - DNA, 1332 bp, Artificial Sequence
  synthetic sequence of chimeric LGIC
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgctacatcgatgtacgctggtttccctttgatgtgcagcactgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatggagcggcagatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctgagatcatgcccgcaacatccgattcggtaccattgatagcccaggccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtga

SEQ ID NO: 27 - protein, 443 aa, Artificial Sequence
  synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMERQMGYYLIQMYIPSLLIVILSWISFWINMDAAPARVGLGITTVLTMTTQSSGSEIMPATSDSVPLIAQAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 28 - DNA, 1332 bp, Artificial Sequence
  synthetic sequence of chimeric LGIC
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgctacatcgatgtacgctggtttccctttgatgtgcagcactgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggacgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctgagatcatgcccgcaacatccgattcggtaccattgatagcccaggccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtga

SEQ ID NO: 29 - protein, 443 aa, Artificial Sequence
  synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRTGYYLIQMYIPSLLIVILSWISFWINMDAAPARVGLGITTVLTMTTQSSGSEIMPATSDSVPLIAQAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 30 - DNA, 1332 bp, Artificial Sequence
  synthetic sequence of chimeric LGIC
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgctacatcgatgtacgctggtttccctttgatgtgcagcactgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggacgctctactacctgattcagatgtatattcccagcctgctcattgtcatcctctcatggatctccttctggatcaacatggatgctgcacctgctcgtgtgggcctaggcatcaccactgtgctcaccatgaccacccagagctccggctctgagatcatgcccgcaacatccgattcggtaccattgatagcccaggccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtga

SEQ ID NO: 31 - protein, 443 aa, Artificial Sequence
  synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRTLYYLIQMYIPSLLIVILSWISFWINMDAAPARVGLGITTVLTMTTQSSGSEIMPATSDSVPLIAQAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 32 - DNA, 1320 bp, Artificial Sequence
  synthetic sequence of chimeric LGIC
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtga

SEQ ID NO: 33 - protein, 439 aa, Artificial Sequence
  synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCPMDLKNFPMDVQTCKLKFGSWSYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 34 - DNA, 1320 bp, Artificial Sequence
  synthetic sequence of chimeric LGIC
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaattttccctttgatgtgcagcactgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtga

SEQ ID NO: 35 - protein, 439 aa, Artificial Sequence
  synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCPMDLKNFPFDVQHCKLKFGSWSYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 36 - DNA, 1320 bp, Artificial Sequence
  synthetic sequence of chimeric LGIC
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagacaaccatggttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgctacatcgatgtacgctggtttccctttgatgtgcagcactgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtga

SEQ ID NO: 37 - protein, 439 aa, Artificial Sequence
  synthetic sequence of chimeric LGIC
MRCSPGGVWLALAASLLHVSLQGEFQRKLYKELVKNYNPLERPVANDSQPLTVYFSLSLLQIMDVDETTMVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 38 - DNA, 1320 bp, Artificial Sequence
  synthetic sequence of chimeric LGIC
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacattgctgagacaaccatggacttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtga

SEQ ID NO: 39 - protein, 439 aa, Artificial Sequence
  synthetic sequence of chimeric LGIC
MRCSPGGVWLALAASLLHVSLQGEFQRKLYKELVKNYNPLERPVANDSQPLTVYFSLSLLQIMDIAETTMDLTTNIWLQMSWTDHYLQWNVSEYPGVKTVRFPDGQIWKPDILLYNSADERFDATFHTNVLVNSSGHCQYLPPGIFKSSCPMDLKNFPMDVQTCKLKFGSWSYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 40 - DNA, 1320 bp, Artificial Sequence
  synthetic sequence of chimeric LGIC
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagacaaccatggttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtga

SEQ ID NO: 41 - protein, 439 aa, Artificial Sequence
  synthetic sequence of chimeric LGIC
MRCSPGGVWLALAASLLHVSLQGEFQRKLYKELVKNYNPLERPVANDSQPLTVYFSLSLLQIMDVDETTMVLTTNIWLQMSWTDHYLQWNVSEYPGVKTVRFPDGQIWKPDILLYNSADERFDATFHTNVLVNSSGHCQYLPPGIFKSSCPMDLKNFPMDVQTCKLKFGSWSYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 42 - DNA, 1317 bp, Artificial Sequence
  synthetic sequence of chimeric LGIC
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagacaaccatggttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatggagcggcagatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccag

SEQ ID NO: 43 - protein, 439 aa, Artificial Sequence
  synthetic sequence of chimeric LGIC
MRCSPGGVWLALAASLLHVSLQGEFQRKLYKELVKNYNPLERPVANDSQPLTVYFSLSLLQIMDVDETTMVLTTNIWLQMSWTDHYLQWNVSEYPGVKTVRFPDGQIWKPDILLYNSADERFDATFHTNVLVNSSGHCQYLPPGIFKSSCPMDLKNFPMDVQTCKLKFGSWSYGGWSLDLQMQEADISGYIPNGEWDLVGIPGKRSERFYECCKEPYPDVTFTVTMERQMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 44 - DNA, 1317 bp, Artificial Sequence
  synthetic sequence of chimeric LGIC
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacattgctgagacaaccatggacttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatggagcggcagatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccag

SEQ ID NO: 45 - protein, 439 aa, Artificial Sequence
  synthetic sequence of chimeric LGIC
MRCSPGGVWLALAASLLHVSLQGEFQRKLYKELVKNYNPLERPVANDSQPLTVYFSLSLLQIMDIAETTMDLTTNIWLQMSWTDHYLQWNVSEYPGVKTVRFPDGQIWKPDILLYNSADERFDATFHTNVLVNSSGHCQYLPPGIFKSSCPMDLKNFPMDVQTCKLKFGSWSYGGWSLDLQMQEADISGYIPNGEWDLVGIPGKRSERFYECCKEPYPDVTFTVTMERQMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 46 - DNA, 1329 bp, Artificial Sequence
  synthetic sequence of chimeric LGIC
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagacaaccatggttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgctacatcgatgtacgctggtttccctttgatgtgcagcactgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctgagatcatgcccgcaacatccgattcggtaccattgatagcccaggccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccag

SEQ ID NO: 47 - protein, 443 aa, Artificial Sequence
  synthetic sequence of chimeric LGIC
MRCSPGGVWLALAASLLHVSLQGEFQRKLYKELVKNYNPLERPVANDSQPLTVYFSLSLLQIMDVDETTMVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSEIMPATSDSVPLIAQAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 48 - DNA, 1332 bp, Artificial Sequence
  synthetic sequence of chimeric LGIC
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctgagatcatgcccgcaacatccgattcggtaccattgatagcccaggccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtga

SEQ ID NO: 49 - protein, 443 aa, Artificial Sequence
  synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCPMDLKNFPMDVQTCKLKFGSWSYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSEIMPATSDSVPLIAQAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 50 - protein, 451 aa, Artificial Sequence
  synthetic sequence of chimeric LGIC
MLLWVQQALLALLLPTLLAQGEARRSRNTTRPALLRLSDYLLTNYRKGVRPVRDWRKPTTVSIDVIVYAILNVDEKNQVLTTYIWYRQYWTDEFLQWNPEDFDNITKLSIPTDSIWVPDILINEFVDVGKSPNIPYVYIRHQGEVQNYKPLQVVTACSLDIYNFPFDVQNCSLTFTSWLHTIQDINISLWRLPEKVKSDRSVFMNQGEWELLGVLPYFREFSMESSNYYAEMKFYVVIRRRMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 51 - protein, 451 aa, Artificial Sequence
  synthetic sequence of chimeric LGIC
MLLWVQQALLALLLPTLLAQGEARRSRNTTRPALLRLSDYLLTNYRKGVRPVRDWRKPTTVSIDVIVYAILNVDEKNQVLTTYIWYRQYWTDEFLQWNPEDFDNITKLSIPTDSIWVPDILINEFVDVGKSPNIPYVYIRHQGEVQNYKPLQVVTACSLDIYNFPFDVQNCSLTFTSWLHTIQDINISLWRLPEKVKSDRSVFMNQGEWELLGVLPYFREFSMESSNYYAEMKFYVHLERQMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 52 - protein, 455 aa, Artificial Sequence
  synthetic sequence of chimeric LGIC
MWGLAGGRLFGIFSAPVLVAVVCCAQSVNDPGNMSFVKETVDKLLKGYDIRLRPDFGGPPVCVGMNIDIASIDMVSEVNMDYTLTMYFQQYWRDKRLAYSGIPLNLTLDNRVADQLWVPDTYFLNDKKSFVHGVTVKNRMIRLHPDGTVLYGLRITTTAACMMDLRRYPLDEQNCTLEIESYGYTTDDIEFYWRGGDKAVTGVERIELPQFSIVEHRLVSRNVVFATGAYPRLSLSFRLKRNIGYMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 53 - protein, 31 aa, Artificial Sequence
  C-terminal amino acids of GABAR1
IDRLSRIAFPLLFGIFNLVYWATYLNREPQL

SEQ ID NO: 54 - protein, 22 aa, Artificial Sequence
  Amino acid residues 260-281 of human CHRNA7
EKISLGITVLLSLTVFMLLVAE

SEQ ID NO: 55 - protein, 23 aa, Artificial Sequence
  Synthetic polypeptide sequence
PAKIGLGITVLLSLTTFMSGVAN

SEQ ID NO: 56 - DNA, 1780 bp, Homo sapiens
atgttgtcaagtgtaatggctcccctgtgggcctgcatcctggtggctgcaggaattctagccacagatacacatcatccccaggattctgctctgtatcatctcagcaagcagctattacagaaatatcataaagaagtgagacctgtttacaactggaccaaggccaccacagtctacctggacctgttcgtccatgctatattggatgtggatgcagagaatcaaatattaaagacaagtgtatggtaccaagaggtctggaatgatgaatttttatcctggaactccagcatgtttgatgagattagagagatctccctacctctaagtgccatctgggcccccgatatcatcatcaatgagtttgtggacattgaaagataccctgaccttccctatgtttatgtgaactcatctgggaccattgagaactataagcccatccaggtggtctctgcgtgcagtttagagacatatgcttttccatttgatgtccagaattgcagcctgaccttcaagagcattctgcatacagtggaagacgtagacctggcctttctgaggagcccagaagacattcagcatgacaaaaaggcgtttttgaatgacagtgagtgggaacttctatctgtgtcctccacatacagcatcctgcagagcagcgctggaggatttgcacagattcagtttaatgtggtgatgcgcaggcaccccctggtctatgtcgtgagtctgctgattcctagcatctttctcatgctggtggacctggggagcttctacctgccacccaactgccgagccaggattgtgttcaagaccagtgtgctggtgggctacaccgtcttcagggtcaacatgtccaaccaggtgccacggagtgtagggagcacccctctgattgggcacttcttcaccatctgcatggccttcttggttctcagcttagctaagtccatcgtgttggtcaaattcctccatgatgagcagcgtggtggacaggagcagcccttcttgtgccttcgaggggacaccgatgctgacaggcctagagtggaacccagggcccaacgtgctgtggtaacagagtcctcgctgtatggagagcacctggcccagccaggaaccctgaaggaagtctggtcgcagcttcaatctatcagcaactacctccaaactcaggaccagacagaccaacaggaggcagagtggctggtcctcctgtcccgctttgaccgactgctcttccaaagctaccttttcatgctggggatctacaccatcactctgtgctccctctgggcactgtggggcggcgtgtgaagactgaagtgttcttcagtaattgtgctggcacttaggagagagaggagggggaataatagtgggttaaaaagctttctgggtcgggtgtggtggttcttgcctatagtcccagtgctttgggaggccatagcaggaggattgcttgagcccaggagttcgagaccagccagagcaacatagtgagaccacatctctaccagtaaataaataaataaataaataaataaataaataaataaatagctgggcatagtggctcatgcctgtactctcagctacttgggaggttgaggtgggaggattgcttgagcccaggatttcaaggctgcagtgagccatgattgcaccactgcaccccagcctgggtgacagagcaagaccctgtctcaaaaaaaataaaataaaaggctttctgccttcaaaaaaaaaaaaaaaaaaaaaaaaaaaaaaa

SEQ ID NO: 57 - protein, 441 aa, Homo sapiens
MLSSVMAPLWACILVAAGILATDTHHPQDSALYHLSKQLLQKYHKEVRPVYNWTKATTVYLDLFVHAILDVDAENQILKTSVWYQEVWNDEFLSWNSSMFDEIREISLPLSAIWAPDIIINEFVDIERYPDLPYVYVNSSGTIENYKPIQVVSACSLETYAFPFDVQNCSLTFKSILHTVEDVDLAFLRSPEDIQHDKKAFLNDSEWELLSVSSTYSILQSSAGGFAQIQFNVVMRRHPLVYVVSLLIPSIFLMLVDLGSFYLPPNCRARIVFKTSVLVGYTVFRVNMSNQVPRSVGSTPLIGHFFTICMAFLVLSLAKSIVLVKFLHDEQRGGQEQPFLCLRGDTDADRPRVEPRAQRAVVTESSLYGEHLAQPGTLKEVWSQLQSISNYLQTQDQTDQQEAEWLVLLSRFDRLLFQSYLFMLGIYTITLCSLWALWGGV

SEQ ID NO: 58 - protein, 439 aa, Artificial Sequence
  synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVDVNSSGHCQYLPPGIFKSSCPMDLKNFPMDVQTCKLKFGSWDYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 59 - protein, 439 aa, Artificial Sequence
  synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVSVNSSGHCQYLPPGIFKSSCPMDLKNFPMDVQTCKLKFGSWDYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 60 - protein, 439 aa, Artificial Sequence
  synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVTVNSSGHCQYLPPGIFKSSCPMDLKNFPMDVQTCKLKFGSWDYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 61 - protein, 439 aa, Artificial Sequence
  synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLDNSADERFDATFHTNVEVNSSGHCQYLPPGIFKSSCPMDLKNFPMDVQTCKLKFGSWSYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 62 - protein, 439 aa, Artificial Sequence
  synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLDNSADERFDATFHTNVQVNSSGHCQYLPPGIFKSSCPMDLKNFPMDVQTCKLKFGSWSYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 63 - protein, 439 aa, Artificial Sequence
  synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLDNSADERFDATFHTNVLVNSSGHCQYLPPGIFKSSCPMDLKNFPMDVQTCKLKFGTWSYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

(19) United States
(12) Patent Application Publication
(10) Pub. No.: US 2022/0348635 A1
(43) Pub. Date: Nov. 3, 2022

(54) COMPOSITIONS AND METHODS FOR NEUROLOGICAL DISEASES

(71) Applicant: Coda Biotherapeutics, Inc., South San Francisco, CA (US)

(72) Inventors: Anthony LAU, Jr., South San Francisco, CA (US); Orion P. KEIFER, Jr., South San Francisco, CA (US); Stefanie MAKINSON, South San Francisco, CA (US)

(21) Appl. No.: 17/636,235

(22) PCT Filed: Aug. 21, 2020
(86) PCT No.: PCT/US2020/047503

§ 371 Date: Feb. 17, 2022

Related U.S. Application Data
(60) Provisional application No. 62/889,963, filed on Aug. 21, 2019.

(51) Int. Cl. C07K 14/705; A61K 48/00; A61P 25/08; A61P 25/16; A61P 25/24; A61P 1/04; A61P 25/22; A61P 25/04

(52) U.S. Cl. CPC C07K 14/70571; C07K 14/705; A61K 48/005; A61P 25/08; A61P 25/16; A61P 25/24; A61P 1/04; A61P 25/22; A61P 25/04; A61K 38/00

(57) ABSTRACT

Compositions and methods are provided for modulating the activity of cells using engineered receptors, polynucleotide encoded engineered receptors, and gene therapy vectors comprising polynucleotides encoding engineered receptors. These compositions and methods find particular use in modulating the activity of neurons, for example in the treatment of disease or in the study of neuronal circuits.

### CROSS REFERENCE TO RELATED APPLICATIONS

This application claims the benefit of the U.S. Provisional Patent Application Ser. No. 62/889,963 filed Aug. 21, 2019, the contents of which are herein incorporated by reference in its entirety.

### DESCRIPTION OF THE TEXT FILE SUBMITTED ELECTRONICALLY

The sequence listing associated with this application is provided in text format in lieu of a paper copy, and is hereby incorporated by reference into the specification. The name of the text file containing the sequence listing is “SWCH02901WO-Sequence_Listing”. The text file is 200 kb, was created on Aug. 21, 2020, and is being submitted electronically via EFS-Web.

### FIELD

This disclosure pertains to engineered receptors and the use of engineered receptors and small molecule ligands to modulate the activity of cells and treat disease.

### BACKGROUND

Intractable neurological disease is often associated with aberrantly acting neurons. Attempts to develop therapies to treat these conditions have been hampered by a lack of tractable target proteins associated with the disease. For example, unrelieved chronic pain is a critical health problem in the US and worldwide. A report by the Institute of Medicine estimated that 116 million Americans suffer from pain that persists for weeks to years, with resulting annual costs exceeding $560 million. There are no adequate long-term therapies for chronic pain sufferers, leading to significant cost for both society and the individual. Pain often results in disability and, even when not disabling, it has a profound effect on the quality of life. Pain treatment frequently fails even when the circumstances of care delivery are optimal, such as attentive, well-trained physicians; ready access to opioids; use of adjuvant analgesics; availability of patient-controlled analgesia; and evidence-based use of procedures like nerve blocks and IT pumps.

The most commonly used therapy for chronic pain is the application of opioid analgesics and nonsteroidal anti-inflammatory drugs, but these drugs can lead to addiction and may cause side effects, such as drug dependence, tolerance, respiratory depression, sedation, cognitive failure, hallucinations, and other systemic side effects. Despite the wide usage of pharmaceuticals, there is a strikingly low success rate for its effectiveness in pain relief. A large randomized study with various medications found only one out of every two or three patients achieving at least 50% pain relief (Finnerup et al., 2005). A follow-up study using the most developed pharmacological treatments found the same results, indicating that there was no improvement in the efficacy of medications for pain (Finnerup et al., Pain, 150(3):573-81, 2010).

More invasive options for the treatment of pain include nerve blocks and electrical stimulation. A nerve block is a local anesthetic injection usually in the spinal cord to interrupt pain signals to the brain, the effect of which only lasts from weeks to months. Nerve blocks are not the recommended treatment option in most cases (Mailis and Taenzer, Pain Res Manag. 17(3):150-158, 2012). Electrical stimulation involves providing electric currents to block pain signals. Although the effect may last longer than a nerve block, complications arise with the electrical leads itself: dislocation, infection, breakage, or the battery dying. One review found that 40% of patients treated with electrical stimulation for neuropathy experienced one or more of these issues with the device (Wolter, 2014).

The most invasive, and least preferred, method for managing pain is complete surgical removal of the nerve or section thereof that is causing the pain. This option is only recommended when the patient has exhausted the former and other less invasive, treatments and found them ineffective. Radiofrequency nerve ablation uses heat to destroy problematic nerves and provides a longer pain relief than a nerve block. However, one study found no difference between the control and treatment groups in partial radiofrequency lesioning of the DRG for chronic lumbosacral radicular pain (Geurts et al., 2003). Other surgical methods for surgically removing the pain nerves suffer from similar shortcomings and have serious side effects long-term, including sensory or motor deficits, or cause pain elsewhere.

Methods for treating neurological disorders should be safe, efficient and cost-effective. Gene therapy could provide non-invasive treatment options for a variety of neurological diseases, including managing pain. However, to date, gene therapy methods have not found widespread use in the treatment of neurological diseases. The present disclosure addresses these needs.

### BRIEF DESCRIPTION OF THE DRAWINGS

The disclosure is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures:

FIGS. 1A-FIG. 1G show the heat maps of the percent quench of YFP fluorescence in the mutants of the engineered chimeric receptor SEQ ID NO:33 that comprise the indicated double amino acid substitutions following stimulation by various doses of either acetylcholine or the indicated non-native ligand. Ligand doses are written across the top of each chart. Numbers in the boxes indicate the absolute amount of quench observed. Dark blue=80% maximal quench of YFP reporter. Light blue=quench of 30-80%. White=10-30%. Orange=0-10% quench. Negative values represent non-responders that have a negative quench due to a stimulation artifact. SEQ ID NO:29 is a non-responding chimera used as a negative control. Abbreviations for non-native ligand names: abt: ABT-126; ach: acetylcholine; apn: APN-1125; azd: AZD-0328; brd: TC-5619; fac: RG3487; tc6: TC-6987.

FIG. 2A-FIG. 2B show the concentration-response curves of CR-11 (Chemogenic receptor-11, an engineered receptor comprising an amino acid sequence having the amino acid substitutions of Y115D and L131Q in SEQ ID NO: 33) expressed in HEK 293 cells to acetylcholine as well as to non-native ligands RG-3487 (SA-2, Synthetic Agonist-2). The responses were evaluated using manual patch clamp electrophysiology. Currents were normalized to unity for the maximal response. The continuous line through the data points is the best fit obtained with the Hill equation, and EC₅₀s for each ligand are estimated from the concentration-response curves. FIG. 2A shows the concentration-response curves of wild type and CR-11 receptors to acetylcholine. FIG. 2B shows the concentration-response curves of wild type and CR-11 receptors to RG-3487 (SA-2).

FIG. 3 shows exemplary chloride currents induced by RG-3487 (SA-2) in adult rat DRG neurons transduced with a Lentivirus expressing CR-11 (Chemogenic receptor-11, an engineered receptor comprising an amino acid sequence having the amino acid substitutions of Y115D and L131Q in SEQ ID NO: 33).

FIG. 4A shows the evoked action potential of transduced DRG neurons expressing CR-11 (an engineered receptor comprising an amino acid sequence having the amino acid substitutions of Y115D and L131Q in SEQ ID NO: 33) or control DRG neurons (without CR-11 expression) at different current injections (50 pA to 700 pA). The upper panel shows the evoked action potential of a control DGF neuron. The lower left panel shows the evoked action potential of a transduced DRG neuron expressing CR-11 in the presence of 3 μM RG-3487 (SA-2). The lower right panel shows the evoked action potential of a transduced DRG neuron expressing CR-11 after RG-3487 (SA-2) is washed away. FIG. 4B shows the rheobase value (current required to elicit action potentials) for the control DRG neurons and transduced DRG neurons expressing CR-11 in the absence or presence of indicated ligand.

FIG. 5 shows the % of HA-tag positive cells that are expressing the engineered receptors, normalized to control cells expressing the amino acid sequence of SEQ ID NO: 33 (“Average HA tag %”); and the % of α-bungarotoxin positive cells that are expressing the engineered receptors, normalized to control cells expressing the amino acid sequence of SEQ ID NO: 33 (“Normalized AB %”). FIG. 5 also shows the median fluorescent intensity (MFI) of a cell expressing the engineered receptors, normalized to control cells expressing the amino acid sequence of SEQ ID NO: 33, as evaluated using anti-HA antibodies (“Average HA MFI”) or fluorescently labeled α-bungarotoxin conjugated to Alexa Fluor 647 (“Normalized AB MFI”).

### SUMMARY

The disclosure provides engineered receptors, comprising: a ligand binding domain derived from the human α7 nicotinic acetylcholine receptor (α7-nAChR) and comprising a Cys-loop domain from the human Glycine receptor α1 subunit; and an ion pore domain derived from the human Glycine receptor α1 subunit. In some embodiments, the engineered receptor is a chimeric ligand gated ion channel (LGIC) receptor. In some embodiments, the ligand binding domain comprises: (i) two amino acid substitutions at a pair of amino acids residues selected from the group consisting of L131 and S172, Y115 and S170, and Y115 and L131; or (ii) an amino acid substitution of L131E, wherein the amino acid residues correspond to the amino acid residues of α7-nAChR. In some embodiments, the engineered receptor comprises an amino acid sequence of SEQ ID NO: 33, wherein the amino acid sequence further comprises the two amino acid substitutions at a pair of amino acids residues selected from the group consisting of L131 and S172, Y115 and S170, and Y115 and L131; or the amino acid substitution of L131E, wherein the amino acid residues correspond to the amino acid residues of α7-nAChR.

In some embodiments, the ligand binding domain comprises two amino acid substitutions at a pair of amino acids residues selected from the group consisting of L131 and S172, Y115 and S170, and Y115 and L131. In some embodiments, the ligand binding domain comprises a pair of amino acid substitutions selected from the group consisting of L131S and S172D, L131T and S172D, L131D and S172D, Y115D and S170T, Y115D and L131Q, and Y115D and L131E. In some embodiments, the engineered receptor comprises an amino acid sequence of SEQ ID NO: 33, wherein the amino acid sequence further comprises a pair of amino acid substitutions selected from the group consisting of L131S and S172D, L131T and S172D, L131D and S172D, Y115D and S170T, Y115D and L131Q, and Y115D and L131E.

In some embodiments, the ligand binding domain comprises an amino acid substitution of L131E. In some embodiments, the engineered receptor comprises an amino acid sequence of SEQ ID NO: 33, wherein the amino acid sequence further comprises an amino acid substitution of L131E.

In some embodiments, the Cys-loop domain comprises amino acids 166-172 of SEQ ID NO: 2. In some embodiments, the Cys-loop domain comprises amino acids 166-180 of SEQ ID NO: 2. In some embodiments, the receptor comprises a β1-2 loop domain from the human Glycine receptor α1 subunit. In some embodiments, the β1-2 loop domain comprises amino acids 81-84 of SEQ ID NO:2. In some embodiments, the engineered receptor comprises an amino acid sequence selected from the group consisting of SEQ ID Nos. 58-63. In some embodiments, the engineered receptor comprises an amino acid sequence selected from the group consisting of SEQ ID NO: 58, SEQ ID NO: 59, SEQ ID NO: 60, SEQ ID NO: 61, SEQ ID NO: 62, and SEQ ID NO: 63.

In some embodiments, the potency of the engineered receptor to acetylcholine is lower than the potency of the human α7 nicotinic acetylcholine receptor (α7-nAChR) to acetylcholine. In some embodiments, the potency of the engineered receptor to acetylcholine is at least 2-fold lower than the potency of the human α7 nicotinic acetylcholine receptor (α7-nAChR) to acetylcholine. In some embodiments, the potency of the engineered receptor to a non-native ligand is about the same as the potency of the human α7 nicotinic acetylcholine receptor (α7-nAChR) to the non-native ligand.

In some embodiments, the potency of the engineered receptor to a non-native ligand is higher than the potency of the human α7 nicotinic acetylcholine receptor (α7-nAChR) to the non-native ligand. In some embodiments, the potency of the engineered receptor to the non-native ligand is at least 2-fold higher than the potency of the human α7 nicotinic acetylcholine receptor (α7-nAChR) to the non-native ligand. In some embodiments, determining the potency comprises determining the EC50.

In some embodiments, the efficacy of the engineered receptor in the presence of a non-native ligand is higher than the efficacy the human α7 nicotinic acetylcholine receptor (α7-nAChR) in presence of the non-native ligand. In some embodiments, the efficacy of the engineered receptor in the presence of a non-native ligand is at least 2-fold higher than the efficacy the human α7 nicotinic acetylcholine receptor (α7-nAChR) in presence of the non-native ligand. In some embodiments, determining the efficacy comprises determining the amount of current passed through the engineered receptor in vitro in the presence of the non-native ligand.

In some embodiments, the non-native ligand is selected from the group consisting of AZD-0328, TC-6987, ABT-126, APN-1125, TC-5619, and Facinicline/RG3487. In some embodiments, the non-native ligand is selected from the group consisting of ABT-126, RG3487, and APN-1125. In some embodiments, the non-native ligand is TC-5619.

The disclosure provides polynucleotides comprising a nucleic acid encoding any one of the engineered receptors disclosed herein. In some embodiments, the polynucleotide comprises a promoter operably linked to the nucleic acid encoding the engineered receptor. In some embodiments, the promoter is a regulatable promoter. In some embodiments, the regulatable promoter is active in an excitable cell. In some embodiments, the excitable cell is a neuron or a myocyte. In some embodiments, the excitable cell is a neuron.

The disclosure provides vectors comprising any one of the polynucleotides disclosed herein. In some embodiments, the vector is a plasmid, or a viral vector. In some embodiments, the vector is a viral vector selected from the group consisting of an adenoviral vector, a retroviral vector, an adeno-associated viral (AAV) vector, and a herpes simplex-1 viral vector (HSV-1). In some embodiments, the viral vector is an AVV vector, and wherein the AAV vector is AAV5 or a variant thereof, AAV6 or a variant thereof or AAV9 or a variant thereof.

The disclosure provides compositions comprising any one of the engineered receptors disclosed herein, any one of the polynucleotides disclosed herein, or any one of the vectors disclosed herein. The disclosure further provides pharmaceutical compositions comprising any one of the engineered receptors disclosed herein, any one of the polynucleotides disclosed herein, or any one of the vectors disclosed herein; and a pharmaceutically acceptable carrier.

The disclosure provides methods of producing an engineered receptor in a neuron, comprising contacting the neuron with any one of the polynucleotides disclosed herein, any one of the vectors disclosed herein, any one of the compositions disclosed herein, or any one of the pharmaceutical compositions disclosed herein. In some embodiments, the neuron is a neuron of the peripheral nervous system. In some embodiments, the neuron is a neuron of the central nervous system. In some embodiments, the neuron is a nociceptive neuron. In some embodiments, the neuron is a non-nociceptive neuron. In some embodiments, the neuron is a dorsal root ganglion (DRG) neuron, a trigeminal ganglion (TG) neuron, a motor neuron, an excitatory neuron, an inhibitory neuron, or a sensory neuron. In some embodiments, the neuron is an Aδ afferent fiber, a C fiber or an Aβ afferent fiber. In some embodiments, the neuron is Aβ afferent fiber. In some embodiments, the Aβ afferent fiber is an injured Aβ afferent fiber. In some embodiments, the Aβ afferent fiber is an uninjured Aβ afferent fiber. In some embodiments, wherein the neuron expresses neurofilament 200 (NF200), piezo 2, and TLR-5. In some embodiments, the neuron does not express TrpV1, prostatic acid phosphatase, NaV1.1.

In some embodiments, the contacting step is performed in vitro, ex vivo, or in vivo. In some embodiments, the contacting step is performed in vivo in a subject. In some embodiments, the contacting step comprises administering the polynucleotide, the vector, the composition, or the pharmaceutical composition to the subject. In some embodiments, the contacting step is performed in vitro or ex vivo. In some embodiments, the contacting step comprises lipofection, nanoparticle delivery, particle bombardment, electroporation, sonication, or microinjection. In some embodiments, the engineered receptor is capable of localizing to the cell surface of the neuron.

The disclosure provides methods of inhibiting the activity of a neuron, comprising (a) contacting the neuron with any one of the engineered receptors disclosed herein, any one of the polynucleotides disclosed herein, any one of the vectors disclosed herein, any one of the compositions disclosed herein, or any one of the pharmaceutical compositions disclosed herein, and (b) contacting the neuron with a non-native ligand of the engineered receptor. In some embodiments, the neuron is a neuron of the peripheral nervous system. In some embodiments, the neuron is a neuron of the central nervous system. In some embodiments, the neuron is a nociceptive neuron. In some embodiments, the neuron is a non-nociceptive neuron. In some embodiments, the neuron is a dorsal root ganglion (DRG) neuron, a trigeminal ganglion (TG) neuron, a motor neuron, an excitatory neuron, an inhibitory neuron, or a sensory neuron. In some embodiments, the neuron is an Aδ afferent fiber, a C fiber or an Aβ afferent fiber. In some embodiments, the neuron is Aβ afferent fiber. In some embodiments, the Aβ afferent fiber is an injured Aβ afferent fiber. In some embodiments, the Aβ afferent fiber is an uninjured Aβ afferent fiber. In some embodiments, the neuron expresses neurofilament 200 (NF200), piezo 2, and TLR-5. In some embodiments, the neuron does not express TrpV1, prostatic acid phosphatase, NaV1.1.

In some embodiments, the contacting step (a) is performed in vitro, ex vivo, or in vivo. In some embodiments, the contacting step (b) is performed in vitro, ex vivo, or in vivo. In some embodiments, the contacting steps (a) and/or (b) are performed in vivo in a subject. In some embodiments, the contacting step (a) comprises administering the engineered receptor, the polynucleotide, the vector, or the pharmaceutical composition to the subject; and/or the contacting step (b) comprises administering the non-native ligand to the subject. In some embodiments, the contacting step (a) and/or (b) comprises lipofection, nanoparticle delivery, particle bombardment, electroporation, sonication, or microinjection. In some embodiments, the engineered receptor is capable of localizing to the cell surface of the neuron.

The disclosure provides methods of treating and/or delaying the onset of a neurological disorder in a subject, in need thereof, comprising: administering to the subject, a therapeutically effective amount of any one of the engineered receptors disclosed herein, any one of the polynucleotides disclosed herein, any one of the vectors disclosed herein, any one of the compositions disclosed herein, or any one of the pharmaceutical compositions disclosed herein, and administering to the subject a non-native ligand of the engineered receptor. In some embodiments, the subject is administered the non-native ligand after step (a). In some embodiments, the subject is administered the non-native ligand concurrently with step (a).

In some embodiments, the neurological disorder is a seizure disorder, a movement disorder, an eating disorder, a spinal cord injury, neurogenic bladder, allodynia, a spasticity disorder, pruritus, Alzheimer's disease, Parkinson's disease, post-traumatic stress disorder (PTSD), gastroesophageal reflux disease (GERD), addiction, anxiety, depression, memory loss, dementia, sleep apnea, stroke, narcolepsy, urinary incontinence, essential tremor, trigeminal neuralgia, burning mouth syndrome, or atrial fibrillation. In some embodiments, the neurological disorder is allodynia. In some embodiments, the non-native ligand is selected from the group consisting of AZD-0328, ABT-126, TC6987, APN-1125, TC-5619, and Facinicline/RG3487.

In some embodiments, the non-native ligand is administered orally, subcutaneously, topically, or intravenously. In some embodiments, the non-native ligand is administered orally. In some embodiments, the engineered receptor, the polynucleotide, the vector, the composition, or the pharmaceutical composition is administered subcutaneously, orally, intrathecally, topically, intravenously, intraganglioncally, intraneurally, intracranially, intraspinally, or to the cisterna magna. In some embodiments, the engineered receptor, the polynucleotide, the vector, the composition, or the pharmaceutical composition is administered by transforaminal injection or intrathecally. In some embodiments, the subject suffers from trigeminal neuralgia, and wherein the engineered receptor, the polynucleotide, the vector, the composition, or the pharmaceutical composition is administered to the trigeminal ganglion (TG) of the subject. In some embodiments, the subject suffers from neuropathic pain, and wherein the engineered receptor, the polynucleotide, the vector, the composition, or the pharmaceutical composition is administered to the dorsal root ganglion (DRG) of the subject. In some embodiments, the subject is a human.

In some embodiments, the therapeutically effectively amount diminishes the severity of a sign and/or or a symptom of the neurological disorder. In some embodiments, the therapeutically effectively amount delays the onset of a sign and/or or a symptom of the neurological disorder. In some embodiments, the therapeutically effectively amount eliminates a sign and/or or a symptom of the neurological disorder. In some embodiments, the sign of the neurological disorder is nerve damage, nerve atrophy, and/or seizure. In some embodiments, the nerve damage is peripheral nerve damage. In some embodiments, the symptom of the neurological disorder is pain.

The disclosure provides methods of treating and/or delaying the onset of pain in a subject, in need thereof, comprising: administering to the subject, a therapeutically effective amount of any one of the engineered receptors disclosed herein, any one of the polynucleotides disclosed herein, any one of the vectors disclosed herein, any one of the compositions disclosed herein, or any one of the pharmaceutical compositions disclosed herein, and administering to the subject a non-native ligand of the engineered receptor. In some embodiments, the subject is administered the non-native ligand after step (a). In some embodiments, the subject is administered the non-native ligand concurrently with step (a). In some embodiments, the non-native ligand is selected from the group consisting of AZD-0328, ABT-126, TC6987, APN-1125, TC-5619, and Facinicline/RG3487.

In some embodiments, the non-native ligand is administered orally, subcutaneously, topically, or intravenously. In some embodiments, the non-native ligand is administered orally. In some embodiments, the engineered receptor, the polynucleotide, the vector, the composition, or the pharmaceutical composition is administered subcutaneously, orally, intrathecally, topically, intravenously, intraganglioncally, intraneurally, intracranially, intraspinally, or to the cisterna magna. In some embodiments, the engineered receptor, the polynucleotide, the vector, the composition, or the pharmaceutical composition is administered by transforaminal injection or intrathecally.

In some embodiments, the subject suffers from trigeminal neuralgia, and wherein the engineered receptor, the polynucleotide, the vector, the composition, or the pharmaceutical composition is administered to the trigeminal ganglion (TG) of the subject. In some embodiments, the subject suffers from neuropathic pain, and wherein the engineered receptor, the polynucleotide, the vector, the composition, or the pharmaceutical composition is administered to the dorsal root ganglion (DRG) of the subject.

In some embodiments, the subject is a human. In some embodiments, the pain is neuropathic pain. In some embodiments, the pain is associated with, caused by, or resulting from chemotherapy. In some embodiments, the pain is associated with, caused by, or resulting from trauma. In some embodiments, the subject suffers from allodynia. In some embodiments, the pain manifests after a medical procedure. In some embodiments, the pain is associated with, is caused by, or resulting from childbirth or Caesarean section. In some embodiments, the pain is associated with, is caused by, or resulting from migraine. In some embodiments, the therapeutically effectively amount diminishes pain in the subject transiently, diminishes pain in the subject permanently, prevents the onset of pain in the subject, and/or eliminates pain in the subject. In some embodiments, steps (a) and (b) are performed before the manifestation of pain in the subject.

### DETAILED DESCRIPTION

### A. Overview

Compositions and methods are provided for modulating the activity of cells using engineered ligand gated ion channel (LGIC) receptors, polynucleotide encoded engineered LGIC receptors, and gene therapy vectors comprising polynucleotides encoding engineered LGIC receptors. These compositions and methods find particular use in modulating the activity of neurons, for example in the treatment of disease or in the study of neuronal circuits. In addition, reagents, devices and kits thereof that find use in practicing the subject methods are provided.

In particular, the present disclosure provides engineered receptors that bind to and signal in response to known drugs, ligands, and/or binding agents. In some embodiments, the engineered receptors described herein demonstrate increased affinity for a known agonist binding agent. In some embodiments, the engineered receptors described herein demonstrate an affinity for an antagonist or modulator binding agent and respond to the antagonist and/or modulator agents as if they were agonist agents. The present disclosure further provides for methods of treating neurological diseases in subjects in need thereof. The present disclosure increases the number of clinical indications that a known drug may be used for by utilizing engineered receptors that respond to a known drug in a manner that is distinct from the wild-type endogenous receptor.

Before the present methods and compositions are described, it is to be understood that this disclosure is not limited to particular method or composition described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

### B. Definitions

As used in this specification and the appended claims, the singular forms “a,” “an” and “the” include plural references unless the content clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

As used in this specification, the term “and/or” is used in this disclosure to either “and” or “or” unless indicated otherwise.

Throughout this specification, unless the context requires otherwise, the word “comprise”, or variations such as “comprises” or “comprising” refers to the inclusion of a stated element or integer or group of elements or integers but not the exclusion of any other element or integer or group of elements or integers. Further, the statement of numerical ranges throughout this specification specifically includes all integers and decimal points in between.

Throughout this specification, unless the context requires otherwise, the phrase “consisting essentially of” refers to a limitation of the scope of composition, method, or kit described to the specified materials or steps that do not materially affect the basic and novel characteristic(s) of the subject disclosure. For example, a ligand binding domain “consisting essentially of” a disclosed sequence has the amino acid sequence of the disclosed sequence plus or minus about 5 amino acid residues at the boundaries of the sequence, e.g. about 5 residues, 4 residues, 3 residues, 2 residues or about 1 residue less than the recited bounding amino acid residue, or about 1 residue, 2 residues, 3 residues, 4 residues, or 5 residues more than the recited bounding amino acid residue.

Throughout this specification, unless the context requires otherwise, the phrase “consisting of” refers to the exclusion from the composition, method, or kit of any element, step, or ingredient not specified in the claim. For example, a ligand binding domain “consisting of” a disclosed sequence consists only of the disclosed amino acid sequence.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “isolated” means material that is substantially or essentially free from components that normally accompany is as found in its native state. In some embodiments, the terms “obtained” or “derived” are used synonymously with isolated.

The terms “subject,” “individual,” and “patient” are used interchangeably herein to refer to a vertebrate, such as a mammal. The mammal may be, for example, a mouse, a rat, a rabbit, a cat, a dog, a pig, a sheep, a horse, a non-human primate (e.g., cynomolgus monkey, chimpanzee), or a human. A subject's tissues, cells, or derivatives thereof, obtained in vivo or cultured in vitro are also encompassed. A human subject may be an adult, a teenager, a child (2 years to 14 years of age), an infant (1 month to 24 months), or a neonate (up to 1 month). In some embodiments, the adults are seniors about 65 years or older, or about 60 years or older. In some embodiments, the subject is a pregnant woman or a woman intending to become pregnant.

The term “sample” refers to a volume and/or mass of biological material that is subjected to analysis. In some embodiments, a sample comprises a tissue sample, cell sample, a fluid sample, and the like. In some embodiments, a sample is taken from or provided by a subject (e.g., a human subject). In some embodiments, a sample comprises a portion of tissue taken from any internal organ, a cancerous, pre-cancerous, or non-cancerous tumor, brain, skin, hair (including roots), eye, muscle, bone marrow, cartilage, white adipose tissue, and/or brown adipose tissue. In some embodiments, a fluid sample comprises buccal swabs, blood, cord blood, saliva, semen, urine, ascites fluid, pleural fluid, spinal fluid, pulmonary lavage, tears, sweat, and the like. Those of ordinary skill in the art will appreciate that, in some embodiments, a “sample” is a “primary sample” in that it is obtained directly from a source (e.g., a subject). In some embodiments, a “sample” is the result of processing of a primary sample, for example to remove certain potentially contaminating components, to isolate certain components, and/or to purify certain components of interest. In some embodiments, a sample is a cell or population of cells (e.g., a neuronal cell). A cell sample may be derived directly from a subject (e.g., a primary sample) or may be a cell line. Cell lines may include non-mammalian cells (e.g., insect cells, yeast cells, and/or bacterial cells) or mammalian cells (e.g., immortalized cell lines).

“Treating” or “treatment” as used herein refers to delivering a composition (e.g., an engineered receptor and/or a binding agent) to a subject and/or population of cells to affect a physiologic outcome. In particular embodiments, treatment results in an improvement (e.g., reduction, amelioration, or remediation) of one or more disease symptoms. The improvement may be an observable or measurable improvement, or may be an improvement in the general feeling of well-being of the subject. Treatment of a disease can refer to a reduction in the severity of disease symptoms. In some embodiments, treatment can refer to a reduction in the severity of disease symptoms to levels comparable to those prior to disease onset. In some embodiments, treatment may refer to a short-term (e.g., temporary or acute) and/or a long-term (e.g., sustained or chronic) reduction in disease symptoms. In some embodiments, treatment may refer to a remission of disease symptoms. In some embodiments, treatment may refer to the prophylactic treatment of a subject at risk of developing a particular disease in order to prevent disease development. Prevention of disease development can refer to complete prevention of the disease symptoms, a delay in disease onset, a lessening of the severity of the symptoms in a subsequently developed disease, or reducing the likelihood of disease development.

As used herein, “management” or “controlling” refers to the use of the compositions or methods contemplated herein, to improve the quality of life for an individual suffering from a particular disease. In certain embodiments, the compositions and methods described herein provide analgesia to a subject suffering from pain.

A “therapeutically effective amount” is an amount of a composition required to achieve a desired therapeutic outcome. The therapeutically effective amount may vary according to factors such as, but not limited to, disease state and age, sex, and weight of the subject. Generally, a therapeutically effective amount is also one in which any toxic or detrimental effects of a composition are outweighed by the therapeutically beneficial effects. A “therapeutically effective amount” includes an amount of a composition that is effective to treat a subject.

An “increase” refers to an increase in a value (e.g., increased binding affinity, increased physiologic response, increased therapeutic effect, etc.) of at least 5% as compared to a reference or control level. For example, an increase may include a 5, 6, 7, 8, 9, 10, 15, 20, 25, 30, 35, 40, 45, 50, 60, 70, 80, 90, 100, 150, 200, 250, 500, 1000% or more increase. Increase also means an increase that is 1.1, 1.2, 1.5, 2, 3, 4, 5, 6, 7, 8, 9, 10, 15, 20, 30 or more times (e.g., 500, 1000 times) higher than a reference or control level.

A “decrease”, “reduce”, “diminish” or synonyms thereof refers to a decrease in a value (e.g., decreased binding affinity, decreased physiologic response, decreased therapeutic effect, decrease in pain in a subject etc.) of at least 5% as compared to a reference or control level. For example, a decrease may include a 5, 6, 7, 8, 9, 10, 15, 20, 25, 30, 35, 40, 45, 50, 60, 70, 80, 90, 100, 150, 200, 250, 500, 1000% or more decrease. Decrease also means a decrease that is 1.1, 1.2, 1.5, 2, 3, 4, 5, 6, 7, 8, 9, 10, 15, 20, 30 or more times (e.g., 500, 1000 times) lower than a reference or control level.

By “maintain,” or “preserve,” or “maintenance,” or “no change,” or “no substantial change,” or “no substantial decrease” refers generally to a physiologic and/or therapeutic effect that is comparable to an effect caused by either vehicle, or a control molecule/composition. A comparable response is one that is not significantly different or measurable different from the reference response

The terms “reference” or “control” level are used interchangeably herein and refer the value of a particular physiologic and/or therapeutic effect in a subject or sample that has not been treated with a composition described herein, or a subject or sample that has been treated with a vehicle control. In some embodiments, a reference level refers to a value of a particular physiologic and/or therapeutic effect that is measure in a subject or sample prior to the administration of a composition described herein (e.g., a baseline level).

As used herein, “ligand” refers to a molecule that binds to another, larger molecule. In some embodiments, the ligand binds to a receptor. In some embodiments, the binding of the ligand to the receptor alters the function of the receptor—to activate or repress its function. In some embodiments, the binding of the ligand to a receptor such a ligand gated ion channel (LGIC) leads to the opening or closing of the ion channel.

“Receptor-ligand binding” and “ligand binding” are used interchangeably herein and refer to the physical interaction between a receptor (e.g., a LGIC) and a ligand. The term “ligand” as used herein may refer to an endogenous or naturally occurring ligand. For example, in some embodiments, a ligand refers to a neurotransmitter (e.g., λ-aminobutyric acid (GABA), acetylcholine, serotonin, and others) and signaling intermediate (e.g., phosphatidylinositol 4,5-bisphosphate (PIP₂)), amino acids (e.g., glycine), or nucleotides (e.g., ATP). In some embodiments, a ligand may refer to a non-native, i.e. synthetic or non-naturally occurring, ligand (e.g., a binding agent). For example, in some embodiments, a ligand refers to a small molecule. Ligand binding can be measured by a variety of methods known in the art (e.g., detection of association with a radioactively labeled ligand).

“Binding affinity” generally refers to the strength of the sum total of noncovalent interactions between a single binding site of a receptor and a ligand. Unless indicated otherwise, as used herein, “binding affinity” refers to intrinsic binding affinity which reflects a 1:1 interaction between members of a binding pair (e.g., receptor and ligand). The affinity of a molecule X for its partner Y can generally be represented by the dissociation constant (K_{d}). Affinity can be measured by common methods known in the art, including those described herein.

The terms “specific binding affinity” or “specific binding” are used interchangeably throughout the specification and claims and refer to binding which occurs between a paired species of molecules, e.g., receptor and ligand. When the interaction of the two species produces a non-covalently bound complex, the binding which occurs is typically electrostatic, hydrogen-bonding, or the result of lipophilic interactions. In various embodiments, the specific binding between one or more species is direct. In one embodiment, the affinity of specific binding is about 2 times greater than background binding (non-specific binding), about 5 times greater than background binding, about 10 times greater than background binding, about 20 times greater than background binding, about 50 times greater than background binding, about 100 times greater than background binding, or about 1000 times greater than background binding or more.

“Signaling” refers to the generation of a biochemical or physiological response as a result of ligand binding to a receptor (e.g., as a result of a binding agent binding to an engineered receptor described herein).

The term “wild type” or “native” is a term of the art understood by skilled persons and means the typical form of an organism, strain, gene, protein, or characteristic as it occurs in nature as distinguished from mutant or variant forms. For example, a wild type protein is the typical form of that protein as it occurs in nature.

The terms “non-native”, “variant”, and “mutant” are used interchangeably throughout the specification and the claims to refer to a mutant of a native, or wild type, composition, for example a variant polypeptide having less than 100% sequence identity with the native, or wild type, sequence.

Amino acid modifications may be amino acid substitutions, amino acid deletions and/or amino acid insertions. Amino acid substitutions may be conservative amino acid substitutions or non-conservative amino acid substitutions. A conservative replacement (also called a conservative mutation, a conservative substitution or a conservative variation) is an amino acid replacement in a protein that changes a given amino acid to a different amino acid with similar biochemical properties (e.g. charge, hydrophobicity and size). As used herein, “conservative variations” refer to the replacement of an amino acid residue by another, biologically similar residue. Examples of conservative variations include the substitution of one hydrophobic residue such as isoleucine, valine, leucine or methionine for another; or the substitution of one polar residue for another, such as the substitution of arginine for lysine, glutamic for aspartic acids, or glutamine for asparagine, and the like. Other illustrative examples of conservative substitutions include the changes of: alanine to serine; arginine to lysine; asparagine to glutamine or histidine; aspartate to glutamate; cysteine to serine; glutamine to asparagine; glutamate to aspartate; glycine to praline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine or leucine, and the like.

The term “parental” or “starter” are used interchangeably throughout the specification and claims to refer to an initial composition, or protein that is mutated, modified, or derivatized, to create an engineered composition having novel properties. In some embodiments, the parental protein is a chimeric protein.

The term “engineered” is used throughout the specification and claims to refer to a non-naturally occurring composition, or protein having properties that are distinct from the parental composition, or protein from which it was derivatized.

In general, “sequence identity” or “sequence homology” refers to the nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (generally nucleotides or amino acids), divided by the total number of symbols in the shorter of the two sequences. The program may be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993). Ranges of desired degrees of sequence identity are approximately 80% to 100% and intervening integer values. Typically, the percent identities between a disclosed sequence and a claimed sequence are at least 80%, at least 85%, at least 90%, at least 95%, or at least 98%.

As used herein, “substantially identical” refers to having a sequence identity that is 85% or more, for example 90% or more, e.g. 95%, 96%, 97%, 98%, 99%, 99.5%, 99.9%, or 100%, wherein the activity of the composition is unaltered by the modifications in the sequence that result in the difference in sequence identity.

As used herein, the term “promoter” refers to one or more nucleic acid control sequences that direct transcription of an operably linked nucleic acid. Promoters may include nucleic acid sequences near the start site of transcription, such as a TATA element. Promoters may also include cis-acting polynucleotide sequences that can be bound by transcription factors. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

As used herein, the terms “virus vector,” “viral vector,” or “gene delivery vector” refer to a virus particle that functions as a nucleic acid delivery vehicle, and which comprises a nucleic acid (e.g., an AAV expression cassette) packaged within a virion. Exemplary virus vectors of the disclosure include adenovirus vectors, adeno-associated virus vectors (AAVs), lentivirus vectors, and retrovirus vectors.

As used herein, “neuronal activity”, “activity of a neuron”, “neuronal firing” and variations and synonyms thereof, refer to the electrical activity resulting from the stimulation or excitation of a neuron. In some embodiments, neuronal activity is measured using automated or manual patch clamp techniques. In some embodiments, determining the activity of a neuron comprises determining the excitatory postsynaptic potential (EPSP), inhibitory postsynaptic potential (IPSP), and/or action potential of the neuron. In some embodiments, the level of activity of a neuron depends on, or is affected by, the excitatory postsynaptic potential (EPSP), inhibitory postsynaptic potential (IPSP), and/or action potential.

As used herein, a “neurological disease” or “neurological disorder” refers to a disease or disorder of the nervous system. In some embodiments, the neurological disease is associated with, caused by, or results from structural, biochemical, and/or electrical abnormalities in the brain, spinal cord, a nerve or any component of the nervous system.

As used herein, a “sign” of a disease refers to a physical or mental feature which is regarded as indicating a condition of disease. In some embodiments, a sign is an objective indication of the disease. In some embodiments, a sign is evaluated, examined, observed or measured objectively by a person other than the patient, such as a doctor.

As used herein, a “symptom” of a disease refers to a physical or mental feature which is regarded as indicating a condition of disease, particularly such a feature that is apparent to the patient. In some embodiments, the symptom is subjectively evaluated by the patient. For example, in some embodiments, the symptom is pain.

As used herein, “potency” refers to the amount of ligand required to produce a certain level of activity of a protein, such as a LGIC. In some embodiments, the activity of the protein, such as LGIC, refers to the opening or closing of the ion channel. In some embodiments, determining the potency comprises determining the half maximal effective concentration (EC50) of the protein, such as a LGIC, to a ligand under specific conditions. The EC50 refers to the concentration of the ligand which induces a response halfway between the baseline and maximum after a specific exposure time.

As used herein, “efficacy” refers to a measure of the activity of a protein, such as a LGIC, in the presence of a ligand. In some embodiments, the efficacy refers to the amount of current passed through the LGIC under specific conditions, such as in the presence of a specific concentration of the ligand. In some embodiments, determining the efficacy comprises determining the amount of current passed through the receptor, and/or the rheobase of the receptor.

As used herein, “responsiveness” refers to a measure of the overall function of a protein, such as a LGIC, in the presence of a ligand. Determining the responsiveness may include the determination and consideration of one or more factors, such as potency, efficacy, and the sub-cellular localization of the protein.

### C. Engineered Receptors

The present disclosure is directed to engineered receptors, engineered receptor mutants, and methods for their use. The term “receptor” as used herein refers to any protein that is situated on the surface of a cell and that can mediate signaling to and/or from the cell. The term “engineered receptor” is used herein to refer to a receptor that has been experimentally altered such that it is physically and/or functionally distinct from a corresponding parental receptor. In some embodiments, the parental receptor is a wild-type receptor. The term “wild-type receptor” is used herein to refer to a receptor having a polypeptide sequence that is identical to the polypeptide sequence of a protein found in nature. Wild-type receptors include receptors that naturally occur in humans as well as orthologs that naturally occur in other eukaryotes, e.g. protist, fungi, plants or animals, for example yeast, insects, nematodes, sponge, mammals, non-mammalian vertebrates. In some embodiments, the parental receptor is a non-native receptor; that is, it is a receptor that does not occur in nature, for example, a receptor that is engineered from a wild type receptor. For example, a parental receptor may be an engineered receptor comprising one or more subunits from one wild-type receptor with one or more subunits from a second wild-type receptor. The resulting proteins are therefore comprised of subunits from two or more wild-type receptors. Therefore, in some embodiments, the parental receptor is a chimeric receptor. Engineered receptors of the present disclosure include, for example, parental receptors, parental receptor mutants, and switch receptors.

In some aspects, an engineered receptor of the present disclosure comprises at least one amino acid mutation relative to the corresponding parental receptor, e.g. one or more mutations in one or more domains of a wild-type receptor. By an “amino acid mutation” it is meant any difference in an amino acid sequence relative to a corresponding parental sequence, e.g. an amino acid substitution, deletion, and/or insertion. In some embodiments, the engineered receptor shares a sequence identity of about 99%, about 98%, about 95%, about 90%, about 85%, about 80%, about 70%, about 60%, about 50%, or less with the corresponding parental receptor, inclusive of all values and subranges that lie therebetween. In some embodiments, the parental receptor mutant has a sequence identity of 85% or more with the corresponding parental receptor, e.g. 90% or more or 95% or more, for example, about 96%, about 97%, about 98% or about 99% identity with the corresponding parental receptor, inclusive of all values and subranges that lie therebetween. In some embodiments, an engineered receptor (e.g., a parental receptor mutant) is generated by error prone PCR.

In some embodiments, the amino acid mutation is a loss-of-function amino acid mutation relative to a corresponding parental receptor. “Loss-of-function” amino acid mutations refer to one or more mutations that reduce, substantially decrease, or abolish the function of the engineered receptor relative to the parental receptor, for example by reducing the binding of an endogenous ligand to an engineered receptor relative to the binding of endogenous ligand to the parental receptor, or by reducing the activity of signaling pathway(s) downstream of the engineered receptor that are typically activated in response to the binding of a binding agent to the corresponding parental receptor.

In some embodiments, the amino acid mutation is a gain-of-function amino acid mutation relative to a corresponding parental receptor. “Gain-of-function” amino acid mutations refer to one or more mutations that modify the function of the engineered receptor relative to the parental receptor, for example by altering or enhancing the affinity of an engineered receptor for a binding agent relative to the binding of endogenous ligand to the parental receptor, or by altering or enhancing the activity of the signaling pathways that are activated in response to the binding of a binding agent to an engineered receptor relative to the binding of the endogenous ligand to the corresponding parental receptor. In some embodiments, a gain-of-function mutation results in an increased affinity of the engineered receptor for a binding agent. In particular embodiments, a gain-of-function mutation results in an increased affinity of the engineered receptor for an agonist binding agent. In some embodiments, a gain-of-function mutation results in an antagonist binding agent acting as an agonist binding agent upon binding to the engineered receptor (e.g., results in the activation of agonist signaling pathways instead of antagonist signaling pathways). In some embodiments, a gain-of-function mutation results in a modulator binding agent acting as an agonist binding agent upon binding to the engineered receptor. In some embodiments, the subject engineered receptor of the present disclosure comprises one or more loss-of-function amino acid mutations and one or more gain-of-function amino acid mutations relative to a corresponding parental receptor.

In some embodiments, the loss of function mutation and the gain of function mutation are at the same residue, i.e. they are the same mutation. In other embodiments, the loss of function mutation and the gain of function mutation are mutations at different amino acid residues. In some embodiments, the subject engineered receptor comprising the loss of function mutation and/or gain of function mutation shares a sequence identity of about 99%, about 98%, about 95%, about 90%, about 85%, about 80%, about 70%, about 60%, about 50%, or less with the corresponding parental receptor, e.g. wild type receptor or non-native receptor. In some embodiments, the subject engineered receptor shares a sequence identity of 85% or more with the corresponding parental receptor, for example 85%, 90%, or 95% or more sequence identity, in some instances 96%, 97%, 98% or more sequence identity, e.g. 99% or 99.5% or more sequence identity, inclusive of all values and subranges that lie therebetween.

In some aspects, engineered receptors of the present disclosure include receptors produced by the combination of one or more amino acid sequences, e.g. subunits, from one wild-type receptor with one or more amino acid sequences, e.g. subunits, from a second wild-type receptor. In other words, the engineered receptor comprises amino acid sequences that are heterologous to one another, where by “heterologous”, it is meant not occurring together in nature. Such receptors are referred to herein as “chimeric receptors”. In some embodiments, chimeric receptors serve as parental receptors from which an engineered receptor of the present disclosure is generated.

In some embodiments, a parental receptor mutant demonstrates increased affinity for an agonist binding agent. In some embodiments, a ligand or a binding agent that functions as an antagonist or modulator when binding to a wild type receptor functions as an agonist when binding to a parental receptor mutant.

In some embodiments, the engineered receptor is a “ligand-gated ion channel” or LGIC. An LGIC refers to a large group of transmembrane proteins that allow passage of ions upon activation by a specific ligand (e.g., chemical or binding agent). LGIC are composed of at least two domains: a ligand binding domain and a transmembrane ion pore domain. Ligand binding to an LGIC results in activation of the LGIC and opening of the ion pore. Ligand binding causes a drastic change in the permeability of the channel to a specific ion or ions; effectively no ions can pass through the channel when it is inactive or closed but up to 10⁷ ions/second can pass through upon ligand binding. In some embodiments, LGICs respond to extracellular ligands (e.g., neurotransmitters) and facilitate an influx of ions into the cytosol. In some embodiments, LGICs respond to intracellular ligands (e.g., nucleotides such at ATP and signaling intermediates such as PIP₂) and facilitate an efflux of ions from the cytosol into the extracellular environment. Importantly, activation of LGIC results in the transport of ions across the cellular membrane (e.g., Ca²⁺, Na⁺, K⁺, Cl⁻, etc.) and does not result in the transport of the ligand itself.

LGIC receptors are comprised of multiple subunits and can be either homomeric receptors or heteromeric receptors. A homomeric receptor is comprised of subunits that are all the same type. A heteromeric receptor is comprised of subunits wherein at least one subunit is different from at least one other subunit comprised within the receptor. For example, the glycine receptor is comprised of 5 subunits of which there are two types: α-subunits, of which there are four isoforms (α₁-α₄) and β-subunits, of which there is a single known isoform. An exemplary homomeric GlyR is a GlyR comprised of 5 α₁-GlyR subunits. Similarly, a homomeric GABA_{A} receptor may be comprised of β₃-GABA_{A} subunits, and an nAchR receptor may be comprised of α₇-nAchR subunits. An exemplary heteromeric GlyR may be comprised of one or more α-subunits and one or more of β-subunits (e.g., an α₁β-GlyR). Subunits of example LGIC receptors are shown in Table 1.

TABLE 1
LGIC Receptors and Subunits

| Receptor | Subunits | Subunit Isoforms |
|---|---|---|
| GlyR | GLRA1 | |
| | GLRA2 | |
| | GLRB | |
| 5HT₃ | 5-HT3A | |
| | 5-HT3B | |
| | 5-HT3C | |
| | 5-HT3D | |
| | 5-HT3E | |
| nAChR | α | α1 |
| | | α2 |
| | | α3 |
| | | α4 |
| | | α5 |
| | | α6 |
| | | α7 |
| | | α8 |
| | | α9 |
| | | α10 |
| | β | β1 |
| | | β2 |
| | | β3 |
| | | β4 |
| | γ | γ |
| | δ | δ |
| | ε | ε |
| GABA_{A} | α | α1 |
| | | α2 |
| | | α3 |
| | | α4 |
| | | α5 |
| GABA_{A} | α | α6 |
| | β | β1 |
| | | β2 |
| | | β3 |
| | γ | γ1 |
| | | γ2 |
| | | γ3 |
| | δ | δ |
| | ε | ε |
| | π | π |
| | ρ | ρ1 |
| | | ρ2 |
| | | ρ3 |
| P2X | P2X1 | |
| | P2X2 | |
| | P2X3 | |
| | P2X4 | |
| | P2X5 | |
| | P2X6 | |
| | P2X7 | |
| KCNQ | α | Kᵥα1 |
| | | Kᵥα2 |
| | | Kᵥα3 |
| | | Kᵥα4 |
| | | Kᵥα5 |
| | | Kᵥα6 |
| | | Kᵥα7 |
| | | Kᵥα8 |
| | | Kᵥα9 |
| | | Kᵥα10 |
| | | Kᵥα11 |
| | | Kᵥα12 |
| | β | Kᵥβ1 |
| | | Kᵥβ2 |
| | | Kᵥβ3 |
| | | minK |
| | | MiRP1 |
| | | MiRP2 |
| | | MiRP3 |
| | | KCNE1-like |
| | | KCNIP1 |
| | | KCNIP2 |
| | | KCNIP3 |
| | | KCNIP4 |

Illustrative examples of families of LGICs suitable for use in particular embodiments include, but are not limited to Cys-loop receptors such as Glycine receptors (GlyR), serotonin receptors (e.g., 5-HT3 receptors), λ-Aminobutyric Acid A (GABA-A) receptors, and Nicotinic acetylcholine receptors (nAchR); as well as Acid-sensing (proton-gated) ion channels (ASICs), Epithelial sodium channels (ENaC), Ionotropic glutamate receptors, IP3 receptor, P2X receptors, the Ryanodine receptor, and Zinc activated channels (ZAC).

Specific non-limiting examples of LGICs that are suitable for use with the methods described herein include: HTR3A; HTR3B; HTR3C; HTR3D; HTR3E; ASIC1; ASIC2; ASIC3; SCNN1A; SCNN1B; SCNN1D; SCNN1G; GABRA1; GABRA2; GABRA3; GABRA4; GABRA5; GABRA6; GABRB1; GABRB2; GABRB3; GABRG1; GABRG2; GABRG3; GABRD; GABRE; GABRQ; GABRP; GABRR1; GABRR2; GABRR3; GLRA1; GLRA2; GLRA3; GLRA4; GLRB; GRIA1; GRIA2; GRIA3; GRIA4; GRID1; GRID2; GRIK1; GRIK2; GRIK3; GRIK4; GRIK5; GRIN1; GRIN2A; GRIN2B; GRIN2C; GRIN2D; GRIN3A; GRIN3B; ITPR1; ITPR2; ITPR3; CHRNA1; CHRNA2; CHRNA3; CHRNA4; CHRNA5; CHRNA6; CHRNA7; CHRNA9; CHRNA10; CHRNB1; CHRNB2; CHRNB3; CHRNB4; CHRNG; CHRND; CHRNE; P2RX1; P2RX2; P2RX3; P2RX4; P2RX5; P2RX6; P2RX7; RYR1; RYR2; RYR3; and ZACN.

TRPV1, TRPM8 and P2X₂ are members of large LGIC families that share structural features as well as gating principles. For example TRPV4, similar to TRPV1, is also triggered by heat, but not by capsaicin; and P2X₃, is triggered by ATP, but desensitizes more rapidly than P2X₂. TRPV1, TRPM8 and P2X₂ are, therefore, non-limiting examples of LGIC suitable for use in particular embodiments.

In one embodiment, the engineered receptor is a TRPV1 or TRPM8 receptor or a mutein thereof. TRPV1 and TRPM8, are vanilloid and menthol receptors expressed by nociceptive neurons of the peripheral nervous system. Both channels are thought to function as non-selective, sodium- and calcium-permeable homotetramers. In addition, both channels and their principal agonists—capsaicin and cooling compounds, such as menthol, respectively—are virtually absent from the central nervous system. Capsaicin and some cooling compounds, including menthol and icilin, contain potential acceptor sites for photolabile blocking groups. Association of a photolabile blocking group with such an acceptor would result in a ligand-gated ion channel in which light acts as an indirect trigger by releasing the active ligand.

In one embodiment, the engineered receptor is a P2X₂ receptor or a mutein thereof. P2X₂ is an ATP-gated non-selective cation channel distinguished by its slow rate of desensitization. P2X₂ may be used as a selectively addressable source of depolarizing current and present a platform for the generation of engineered channel-ligand combinations that lack natural agonists altogether.

Non-limiting examples of sequences of wild-type LGIC receptor that find use in the generation of engineered receptors of the present disclosure include the following. In the sequences, the signal peptide is italicized, the ligand binding domain is bolded, and the ion pore domain is underlined:

In some embodiments, the wild-type LGIC receptor is a human alpha 1 glycine receptor (GlyRα1) (GenBank Accession No. NP 001139512.1, SEQ ID NO:2), encoded by the GLRA1 gene (GenBank Accession No. NM_001146040.1 (SEQ ID NO:1):

(SEQ ID NO: 2)
(a) MYSFNTLRLYLWETIVFFSLAASKEAEAARSAPKPMSPSDFLDKLM
GRTSGYDARIRPNFKGPPVNVSCNIFINSFGSIAETTMDYRVNIFLRQQW
NDPRLAYNEYPDDSLDLDPSMLDSIWKPDLFFANEKGAHFHEITTDNKLL
RISRNGNVLYSIRITLTLACPMDLKNFPMDVQTCIMQLESFGYTMNDLIF
EWQEQGAVQVADGLTLPQFILKEEKDLRYCTKHYNTGKFTCIEARFHLER
QMGYYLIQMYIPSLLIVILSWISFWINMDAAPARVGLGITTVLTMTTQSS
GSRASLPKVSYVKAIDIWMAVCLLFVFSALLEYAAVNFVSRQHKELLRFR
RKRRHHKSPMLNLFQEDEAGEGRFNFSAYGMGPACLQAKDGISVKGANNS
NTTNPPPAPSKSPEEMRKLFIQRAKKIDKISRIGFPMAFLIFNMFYWIIY
KIVRREDVHNQ.

In some embodiments, the wild-type LGIC receptor is a human nicotinic cholinergic receptor alpha 7 subunit (α7-nAchR) (GenBank Accession No. NP_000737.1, SEQ ID NO:4), encoded by the CHRNA7 gene (GenBank Accession No. NM_000746.5 (SEQ ID NO:3):

(SEQ ID NO: 4)
(a) MRCSPGGVWLALAASLLHVSLQGEFQRKLYKELVKNYNPLERPVAN
DSQPLTVYFSLSLLQIMDVDEKNQVLTTNIWLQMSWTDHYLQWNVSEYPG
VKTVRFPDGQIWKPDILLYNSADERFDATFHTNVLVNSSGHCQYLPPGIF
KSSCYIDVRWFPFDVQHCKLKFGSWSYGGWSLDLQMQEADISGYIPNGEW
DLVGIPGKRSERFYECCKEPYPDVTFTVTMRRRTLYYGLNLLIPCVLISA
LALLVFLLPADSGEKISLGITVLLSLTVFMLLVAEIMPATSDSVPLIAQY
FASTMIIVGLSVVVTVIVLQYHHHDPDGGKMPKWTRVILLNWCAWFLRMK
RPGEDKVRPACQHKQRRCSLASVEMSAVAPPPASNGNLLYIGFRGLDGVH
CVPTPDSGVVCGRMACSPTHDEHLLHGGQPPEGDPDLAKILEEVRYIANR
FRCQDESEAVCSEWKFAACVVDRLCLMAFSVFTIICTIGILMSAPNFVEA
VSKDFA.

In some embodiments, the wild-type LGIC receptor is a human 5-hydroxytryptamine receptor 3A (5HT3A, GenBank Accession No. NP_998786.2, SEQ ID NO:6), encoded by the HTR3A gene (GenBank Accession No. NM_213621.3, SEQ ID NO:5):

(SEQ ID NO: 6)
(a) MLGKLAMLLWVQQALLALLLPTLLAQGEARRSRNTTRPALLRLSDY
LLTNYRKGVRPVRDWRKPTTVSIDVIVYAILNVDEKNQVLTTYIWYRQYW
TDEFLQWNPEDFDNITKLSIPTDSIWVPDILINEFVDVGKSPNIPYVYIR
HQGEVQNYKPLQVVTACSLDIYNFPFDVQNCSLTFTSWLHTIQDINISLW
RLPEKVKSDRSVFMNQGEWELLGVLPYFREFSMESSNYYAEMKFYVVIRR
RPLFYVVSLLLPSIFLMVMDIVGFYLPPNSGERVSFKITLLLGYSVFLII
VSDTLPATAIGTPLIGKAPPGSRAQSGEKPAPSHLLHVSLASALGCTGVY
FVVCMALLVISLAETIFIVRLVHKQDLQQPVPAWLRHLVLERIAWLLCLR
EQSTSQRPPATSQATKTDDCSAMGNHCSHMGGPQDFEKSPRDRCSPPPPP
REASLAVCGLLQELSSIRQFLEKRDEIREVARDWLRVGSVLDKLLFHIYL
LAVLAYSITLVMLWSIWQYA.

In some embodiments, the wild-type LGIC receptor is a human 5-hydroxytryptamine receptor 3B (5HT3B GenBank Accession No. NP 006019.1, SEQ ID NO:57), encoded by the HTR3B gene (GenBank Accession No. NM_006028.4, SEQ ID NO:56):

(SEQ ID NO: 57)
(a) MLSSVMAPLWACILVAAGILATDTHHPQDSALYHLSKQLLQKYHKE
VRPVYNWTKATTVYLDLFVHAILDVDAENQILKTSVWYQEVWNDEFLSWN
SSMFDEIREISLPLSAIWAPDIIINEFVDIERYPDLPYVYVNSSGTIENY
KPIQVVSACSLETYAFPFDVQNCSLTFKSILHTVEDVDLAFLRSPEDIQH
DKKAFLNDSEWELLSVSSTYSILQSSAGGFAQIQFNVVMRRHPLVYVVSL
LIPSIFLMLVDLGSFYLPPNCRARIVFKTSVLVGYTVFRVNMSNQVPRSV
GSTPLIGHFFTICMAFLVLSLAKSIVLVKFLHDEQRGGQEQPFLCLRGDT
DADRPRVEPRAQRAVVTESSLYGEHLAQPGTLKEVWSQLQSISNYLQTQD
QTDQQEAEWLVLLSRFDRLLFQSYLFMLGIYTITLCSLWALWGGV.

In some embodiments, the wild-type LGIC receptor is a human Gamma-aminobutyric acid receptor A (GABA-A), subunit beta-3 (GABA-A β3) (GenBank Accession No. NP 000805.1, SEQ ID NO:8), encoded by the GABRB3 gene (GenBank Accession No. NM_000814.5, SEQ ID NO:7):

(SEQ ID NO: 8)
(a) MWGLAGGRLFGIFSAPVLVAVVCCAQSVNDPGNMSFVKETVDKLLK
GYDIRLRPDFGGPPVCVGMNIDIASIDMVSEVNMDYTLTMYFQQYWRDKR
LAYSGIPLNLTLDNRVADQLWVPDTYFLNDKKSFVHGVTVKNRMIRLHPD
GTVLYGLRITTTAACMMDLRRYPLDEQNCTLEIESYGYTTDDIEFYWRGG
DKAVTGVERIELPQFSIVEHRLVSRNVVFATGAYPRLSLSFRLKRNIGYF
ILQTYMPSILITILSWVSFWINYDASAARVALGITTVLTMTTINTHLRET
LPKIPYVKAIDMYLMGCFVFVFLALLEYAFVNYIFFGRGPQRQKKLAEKT
AKAKNDRSKSESNRVDAHGNILLTSLEVHNEMNEVSGGIGDTRNSAISFD
NSGIQYRKQSMPREGHGRFLGDRSLPHKKTHLRRRSSQLKIKIPDLTDVN
AIDRWSRIVFPFTFSLFNLVYWLYYVN.

In some embodiments, the wild-type LGIC receptor is a human GABA-A, subunit rho1 (ρ1) (GABA-A ρ1) (GenBank Accession No. NP_002033.2, SEQ ID NO:10), encoded by the GABRR1 gene (GenBank Accession No. NM_002042.4, SEQ ID NO:9):

(SEQ ID NO: 10)
(a) MLAVPNMRFGIFLLWWGWVLATESRMHWPGREVHEMSKKGRPQRQR
REVHEDAHKQVSPILRRSPDITKSPLTKSEQLLRIDDHDFSMRPGFGGPA
IPVGVDVQVESLDSISEVDMDFTMTLYLRHYWKDERLSFPSTNNLSMTFD
GRLVKKIWVPDMFFVHSKRSFIHDTTTDNVMLRVQPDGKVLYSLRVTVTA
MCNMDFSRFPLDTQTCSLEIESYAYTEDDLMLYWKKGNDSLKTDERISLS
QFLIQEFHTTTKLAFYSSTGWYNRLYINFTLRRHIFFFLLQTYFPATLMV
MLSWVSFWIDRRAVPARVPLGITTVLTMSTIITGVNASMPRVSYIKAVDI
YLWVSFVFVFLSVLEYAAVNYLTTVQERKEQKLREKLPCTSGLPPPRTAM
LDGNYSDGEVNDLDNYMPENGEKPDRMMVQLTLASERSSPQRKSQRSSYV
SMRIDTHAIDKYSRIIFPAAYILFNLIYWSIFS.

In some embodiments, the wild-type LGIC receptor is a human GABA-A, subunit rho2 (ρ2) (GABA-A ρ2) (GenBank Accession No. NP_002034.3, SEQ ID NO:12), encoded by the GABRR2 gene (GenBank Accession No. NM_002043.4, SEQ ID NO:11):

(a)
(SEQ ID NO: 12)
MPYFTRLILFLFCLMVLVESRKPKRKRWTGQVEMPKPSHLYKKNLDVTKI
RKGKPQQLLRVDEHDFSMRPAFGGPAIPVGVDVQVESLDSISEVDMDFTM
TLYLRHYWKDERLAFSSASNKSMTFDGRLVKKIWVPDVFFVHSKRSFTHD
TTTDNIMLRVFPDGHVLYSMRITVTAMCNMDFSHFPLDSQTCSLELESYA
YTDEDLMLYWKNGDESLKTDEKISLSQFLIQKFHTTSRLAFYSSTGWYNR
LYINFTLRRHIFFFLLQTYFPATLMVMLSWVSFWIDRRAVPARVSLGITT
VLTMTTIITGVNASMPRVSYVKAVDIYLWVSFVFVFLSVLEYAAVNYLTT
VQERKERKLREKFPCMCGMLHSKTMMLDGSYSESEANSLAGYPRSHILTE
EERQDKIVVHLGLSGEANAARKKGLLKGQTGFRIFQNTHAIDKYSRLIFP
ASYIFFNLIYWSVFS.

In some embodiments, the wild-type LGIC receptor is a human GABA-A, subunit rho3 (ρ3) (GABA-A ρ3) (GenBank Accession No. NP_001099050.1, SEQ ID NO: 14), encoded by the GABRR3 gene (GenBank Accession No. NM_001105580.2, SEQ ID NO:13):

(a)
(SEQ ID NO: 14)
MVLAFQLVSFTYIWIILKPNVCAASNIKMTHQRCSSSMKQTCKQETRMKK
DDSTKARPQKYEQLLHIEDNDFAMRPGFGGSPVPVGIDVHVESIDSISET
NMDFTMTFYLRHYWKDERLSFPSTANKSMTFDHRLTRKIWVPDIFFVHSK
RSFIHDTTMENIMLRVHPDGNVLLSLRITVSAMCFMDFSRFPLDTQNCSL
ELESYAYNEDDLMLYWKHGNKSLNTEEHMSLSQFFIEDFSASSGLAFYSS
TGWYNRLFINFVLRRHVFFFVLQTYFPAILMVMLSWVSFWIDRRAVPARV
SLGITTVLTMSTIITAVSASMPQVSYLKAVDVYLWVSSLFVFLSVIEYAA
VNYLTTVEERKQFKKTGKISRMYNIDAVQAMAFDGCYHDSEIDMDQTSLS
LNSEDFMRRKSICSPSTDSSRIKRRKSLGGHVGRIILENNHVIDTYSRIL
FPIVYILFNLFYWGVYV.

In some aspects, the subject engineered receptor is a chimeric receptor. In some embodiments, the chimeric receptor comprises a ligand binding domain sequence from at least at first LGIC and an ion pore conduction domain sequence, or more simply, “ion pore domain sequence” from a least a second LGIC. In some embodiments, the first and second LGIC are Cys-loop receptors. Ligand binding domain sequences and ion pore domain sequences of the Cys-loop receptors are well known in the art and can be readily identified from the literature by use of publicly available software, e.g. PubMed, Genbank, Uniprot, and the like. In the sequences described above, the ligand binding domain is bolded, and the ion pore domain is underlined.

In some embodiments, the ligand binding domain of the chimeric receptor comprises the ligand binding domain sequence of a human glycine receptor. In some embodiments, the human glycine receptor is human GlyRα1 (SEQ ID NO:2). In some such embodiments, the ligand binding domain comprises about amino acids 29-235 of GlyRα1, e.g. amino acids 29-235, amino acids 29-240, amino acids 29-246, amino acids 29-248, amino acids 29-250, or amino acids 29-252 of SEQ ID NO:2. In certain such embodiments, the ligand binding domain consists essentially of amino acids 29-235 of SEQ ID NO:2, consists essentially of amino acids 29-240 of SEQ ID NO:2, consists essentially of amino acids 29-246 of SEQ ID NO:2, consists essentially of amino acids 29-248 of SEQ ID NO:2, consists essentially of amino acids 29-250 of SEQ ID NO:2, consists essentially of amino acids 29-252 of SEQ ID NO:2. In some embodiments, the ion pore domain sequence is from a Cys-loop receptor other than the human GlyRα1.

In some embodiments, the ligand binding domain of the chimeric receptor comprises the ligand binding domain sequence of a human nicotinic cholinergic receptor. In some embodiments, the human nicotinic cholinergic receptor is human α7-nAChR. In some such embodiments, the ligand binding domain comprises about amino acids 23-220 of α7-nAChR (SEQ ID NO:4), e.g. amino acids 23-220, amino acids 23-226, amino acids 23-229, amino acids 23-230, in some instances amino acids 23-231 of SEQ ID NO:4. In certain such embodiments, the ligand binding domain consists essentially of amino acids 23-220 of SEQ ID NO:4, consists essentially of amino acids 23-226 of SEQ ID NO:4, consists essentially of amino acids 23-229 of SEQ ID NO:4, consists essentially of amino acids 23-230 of SEQ ID NO:4, or consists essentially of amino acids 23-231 of SEQ ID NO:4. In some embodiments, the ion pore domain sequence is from a Cys-loop receptor other than the human α7-nAChR.

In some embodiments, the ligand binding domain of the chimeric receptor comprises the ligand binding domain sequence of a human serotonin receptor. In some embodiments, the human serotonin receptor is human 5HT3A or 5HT3B. In some such embodiments, the ligand binding domain comprises about amino acids 23-247 of 5HT3A (SEQ ID NO:6), e.g. amino acids 23-240, amino acids 30-245, amino acids 23-247, amino acids 23-250, in some instances amino acids 30-255 of SEQ ID NO:6. In certain embodiments, the ligand binding domain consists essentially of amino acids 23-240 of SEQ ID NO:6, consists essentially of amino acids 23-245 of SEQ ID NO:6, consists essentially of amino acids 30-247 of SEQ ID NO:6, consists essentially of amino acids 23-250 of SEQ ID NO:6, consists essentially of amino acids 23-255 of SEQ ID NO:6. In some such embodiments, the ligand binding domain comprises about amino acids 21-239 of 5HT3B (SEQ ID NO:57), e.g. amino acids 21-232, amino acids 21-235, amino acids 21-240, amino acids 21-245, in some instances amino acids 21-247 of SEQ ID NO:57. In certain embodiments, the ligand binding domain consists essentially of amino acids 21-239 of SEQ ID NO:57, consists essentially of amino acids 21-232 of SEQ ID NO:57, consists essentially of amino acids 21-235 of SEQ ID NO:57, consists essentially of amino acids 21-240 of SEQ ID NO:57, consists essentially of amino acids 21-245 of SEQ ID NO:57. In some embodiments, the ion pore domain sequence is from a Cys-loop receptor other than the human 5-hydroxytryptamine receptor 3.

In some embodiments, the ligand binding domain of the chimeric receptor comprises the ligand binding domain sequence of a human GABA receptor. In some embodiments, the human GABA receptor is human GABA-A (33. In some such embodiments, the ligand binding domain comprises about amino acids 26-245 of GABA-A β3 (SEQ ID NO:8), e.g. amino acids 26-240, amino acids 26-245, amino acids 26-248, amino acids 26-250, in some instances amino acids 26-255 of SEQ ID NO:8. In certain such embodiments, the ligand binding domain consists essentially of amino acids 26-240 of SEQ ID NO:8, consists essentially of amino acids 26-245 of SEQ ID NO:8, consists essentially of amino acids 26-248 of SEQ ID NO:8, consists essentially of amino acids 26-250 of SEQ ID NO:8, or consists essentially of amino acids 26-255 of SEQ ID NO:8. In some embodiments, the ion pore domain sequence is from a Cys-loop receptor other than the human GABA-A receptor.

In some embodiments, the ion pore domain to which the ligand binding domain is fused conducts anions, e.g. it comprises an ion pore domain sequence of a human glycine receptor or a human serotonin receptor. In other embodiments, the ion conduction pore domain to which the ligand binding domain is fused conducts cations, e.g. it comprises an ion pore domain sequence of a human acetylcholine receptor or a human gamma-aminobutyric acid receptor A.

In some embodiments, the ion pore domain comprises the ion pore domain sequence of a human glycine receptor. In some embodiments, the human glycine receptor is human GlyRα1. In some such embodiments, the ion pore domain comprises about amino acids 245-457 of GlyRα1 (SEQ ID NO:2), e.g. amino acids 240-457, amino acids 245-457, amino acids 248-457, amino acids 249-457, amino acids 250-457, amino acids 255-457, or amino acids 260-457 of SEQ ID NO:2. In certain such embodiments, the ion pore domain consists essentially of amino acids 245-457 of SEQ ID NO:2, consists essentially of amino acids 248-457 of SEQ ID NO:2, consists essentially of amino acids 249-457 of SEQ ID NO:2, or consists essentially of amino acids 250-457 of SEQ ID NO:2.

In some embodiments, the ion pore domain comprises the ion pore domain sequence of a human nicotinic cholinergic receptor. In some embodiments, the human nicotinic cholinergic receptor is human α7-nAChR. In some such embodiments, the ion pore domain comprises about amino acids 230-502 of α7-nAChR (SEQ ID NO:4), e.g. amino acids 227-502, amino acids 230-502, amino acids 231-502, amino acids 232-502, or amino acids 235-502. In certain such embodiments, the ion pore domain consists essentially of amino acids 227-502 of SEQ ID NO:4, consists essentially of amino acids 230-502 of SEQ ID NO:4, consists essentially of amino acids 231-502 of SEQ ID NO:4, consists essentially of amino acids 232-502 of SEQ ID NO:4, or consists essentially of amino acids 235-502 of SEQ ID NO:4.

In some embodiments, the ion pore domain comprises the ion pore domain sequence of a human serotonin receptor. In some embodiments, the human serotonin receptor is human 5HT3A or 5HT3B. In some such embodiments, the ion pore domain comprises about amino acids 248-516 of 5HT3A (SEQ ID NO:6), e.g. amino acids 240-516, amino acids 245-516, amino acids 248-516, amino acids 250-516, or amino acids 255-516 of SEQ ID NO:6. In certain such embodiments, the ion pore domain consists essentially of amino acids 240-516 of SEQ ID NO:6, consists essentially of amino acids 245-516 of SEQ ID NO:6, consists essentially of amino acids 248-516 of SEQ ID NO:6, consists essentially of amino acids 250-516 of SEQ ID NO:6, or consists essentially of amino acids 253-516. In some such embodiments, the ion pore domain comprises about amino acids 240-441 of 5HT3B (SEQ ID NO:57), e.g. amino acids 230-441, amino acids 235-441, amino acids 240-441, amino acids 245-441, or amino acids 250-441 of SEQ ID NO:57. In certain such embodiments, the ion pore domain consists essentially of amino acids 230-441 of SEQ ID NO:57, consists essentially of amino acids 235-441 of SEQ ID NO:57, consists essentially of amino acids 240-441 of SEQ ID NO:57, consists essentially of amino acids 245-441 of SEQ ID NO:57, or consists essentially of amino acids 250-441.

In some embodiments, the ion pore domain comprises the ion pore domain sequence of a human GABA receptor. In some embodiments, the human GABA receptor is human GABA-A 133. In some such embodiments, the ion pore domain comprises about amino acids 246-473 of GABA-A (SEQ ID NO:8), e.g. amino acids 240-473, amino acids 245-473, amino acids 247-473, amino acids 250-473, or amino acids 253-473 of SEQ ID NO:8. In certain such embodiments, the ion pore domain consists essentially of amino acids 240-473 of SEQ ID NO:8, amino acids 245-473 of SEQ ID NO:8, amino acids 247-473 of SEQ ID NO:8, amino acids 250-473 of SEQ ID NO:8, or amino acids 253-473 of SEQ ID NO:8.

In some embodiments, the ion pore domain of the subject chimeric ligand-gated ion channel comprises an M2-M3 linker domain that is heterologous to the M2-M3 linker domain of the ion pore domain. By an “M2-M3 linker domain”, or “M2-M3 linker”, it is meant the sequence within an ion pore domain of a LGIC that is flanked at its amino (N) terminus by the C-terminal end of transmembrane domain 2 (M2) of the receptor and at its carboxy (C) terminus by the N-terminal end of transmembrane domain 3 (M3) of the receptor. The M2-M3 linker of a LGIC may be readily determined from the art and/or by using any publicly available protein analysis tool, e.g. Expasy, uniProt, etc. Typically, when the ion pore domain of a chimeric receptor comprises a heterologous M2-M3 linker, the M2-M3 linker is derived from the same receptor as the ligand binding domain of the chimeric receptor. For example, when the subject ligand-gated ion channel comprises a ligand binding domain from an AChR and an ion pore domain from a GlyR, the subject ligand-gated ion channel may comprise an ion pore domain sequence from a GlyR except for the M2-M3 linker, which would instead be derived from a AChR. In some embodiments, the ion pore domain is from GlyRα1 and the M2-M3 linker is from α7-nAChR. In some such embodiments, the M2-M3 linker sequence that is removed from the GlyRα1 is about amino acids 293-311 of GlyRα1 (SEQ ID NO:2), e.g. amino acids 304-310, 293-306, 298-310, 305-311, etc. In some such embodiments, the M2-M3 linker that is inserted is about amino acids 281-295 of α7-nAChR (SEQ ID NO:4), e.g. amino acids 290-295, 281-290, 281-295, 287-292, etc. or a sequence having about 95% identity or more to amino acids 281-295 of α7-nAChR.

In some embodiments, the ligand binding domain of the subject chimeric ligand-gated ion channel comprises a Cys-loop domain sequence that is heterologous to the Cys-loop sequence of the ligand binding domain. By a “Cys-loop domain sequence”, or “Cys-loop sequence”, it is meant the domain within a ligand binding domain of a Cys-loop LGIC that forms a loop structure flanked by a cysteine at the N-terminus and the C-terminus. Without wishing to be bound by theory, it is believed that upon binding of the ligand to the ligand binding domain, the Cys-loop structurally moves to be in close proximity to the M2-M3 loop, this movement mediating the biophysical translation of ligand binding in the extracellular domain to signal transduction in the ion pore domain (as reviewed in Miller and Smart, Trends in Pharmacological Sci 2009:31(4)). The substitution of an endogenous Cys-loop sequence with a heterologous Cys-loop sequence may increase the conductivity of the LGIC by 1.5-fold or more, e.g. at least 2-fold, 3-fold or 4-fold, in some instances at least 5-fold or 6-fold, and at certain doses, at least 7-fold, 8-fold, 9-fold or 10-fold. The Cys-loop domain of a Cys-loop receptor may be readily determined from the art and/or by using any publicly available protein analysis tool, e.g. Expasy, uniProt, etc. Typically, when the ligand binding domain of a chimeric receptor comprises a heterologous Cys-loop sequence, the Cys-loop sequence is derived from the same receptor as the ion pore domain of the chimeric receptor. For example, when the subject chimeric ligand-gated ion channel comprises a ligand binding domain from an AChR and an ion pore domain from a GlyR, the subject ligand-gated ion channel may comprise ligand binding domain sequence from an AChR except for the sequence of the Cys-loop domain, which is instead derived from a GlyR. In some embodiments, the ligand binding domain is from α7-nAChR and the Cys-loop sequence is from GlyRα1. In some such embodiments, the Cys-loop sequence that is removed from the α7-nAChR is about amino acids 150-164 of α7-nAChR (SEQ ID NO:4), e.g. amino acids 150-157 of α7-nAChR. In some such embodiments, the Cys loop sequence that is inserted is about amino acids 166-180 of GlyRα1 (SEQ ID NO:2), e.g. amino acids 166-172 of GlyRα1, or a sequence having about 95% identity or more to amino acids 166-180 of GlyRα1.

In some embodiments, the ligand binding domain of the subject chimeric ligand-gated ion channel comprises a β1-2 loop domain sequence that is heterologous to the β1-2 loop domain sequence of the ligand binding domain. By a “β1-2 loop domain sequence”, or “β1-2 loop, or β1-β2 loop”, it is meant the domain within a ligand binding domain of a Cys-loop LGIC that is flanked at its N-terminus by the C-terminus of the β1 sheet and, at its C-terminus, by the N-terminus of the β2 sheet. Without wishing to be bound by theory, it is believed that the β1-2 loop helps to mediate biophysical translation of ligand binding in the extracellular domain to the ion pore domain and subsequent signal transduction (i.e. chloride influx in case of GlyR). It is believed that upon binding of ligand, the β1-2 loop, together with the Cys-loop, come in close proximity to the M2-M3 loop to mediate the biophysical translation of ligand binding in the extracellular domain to signal transduction in the ion pore domain where the M2-M3 loop resides (as reviewed in Miller and Smart, supra). The substitution of an endogenous β1-2 loop sequence with a heterologous β1-2 loop sequence may increase the conductivity of the LGIC by 1.5-fold or more, e.g. at least 2-fold, 3-fold or 4-fold, in some instances at least 5-fold or 6-fold, and at certain doses, at least 7-fold, 8-fold, 9-fold or 10-fold. The β1-2 loop of a Cys-loop receptor may be readily determined from the art and/or by using any publicly available protein analysis tool, e.g. Expasy, uniProt, etc. Typically, when the ligand binding domain of a chimeric receptor comprises a heterologous β1-2 loop sequence, the β1-2 loop sequence is derived from the same receptor as the ion pore domain of the chimeric receptor. For example, when the subject chimeric ligand-gated ion channel comprises a ligand binding domain from an AChR and an ion pore domain from a GlyR, the subject ligand-gated ion channel may comprise ligand binding domain sequence from an AChR except for the sequence of the β1-2 loop domain, which is instead derived from a GlyR. In some embodiments, the ligand binding domain is from α7-nAChR and the β1-2 loop sequence is from GlyRα1. In some embodiments, the β1-2 loop sequence that is removed from the α7-nAChR is about amino acids 67-70 of α7-nAChR (SEQ ID NO:4), e.g. amino acids 67-70, 66-71 or 64-72 of α7-nAChR. In some embodiments, the β1-2 loop sequence that is inserted is about amino acids 79-85 of GlyRα1 (SEQ ID NO:2), e.g. amino acids 81-84, 79-85, or 81-84 of GlyRα1, or a sequence having about 95% identity or more to amino acids 79-85 of GlyRα1.

Non-limiting examples of sequences of chimeric LGIC receptors of the present disclosure include the sequences disclosed herein as SEQ ID NO:15-SEQ ID NO:52. In some embodiments, the chimeric LGIC receptor or the polynucleotide that encodes it has a sequence identity of 85% or more to a sequence provided in SEQ ID NO:15-SEQ ID NO:52 herein, e.g. a sequence identity of 90% or more, 93% or more, or 95% or more, i.e. about 96%, about 97%, about 98%, about 99% or about 100% to a sequence provided in SEQ ID NO:15-SEQ ID NO:52. In the sequences, the signal peptide is italicized, the ligand binding domain is bolded, and the ion pore domain is underlined.

In some embodiments, the chimeric LGIC receptor is a CHRNA7/GLRA1 chimera (R229 junction), comprising the human α7-nAChR signal peptide (italics) and ligand binding domain (bold), fused to the human GlyRα1 ion pore domain (underlined):

(SEQ ID NO: 16, encoded by SEQ ID NO: 15)
MRCSPGGVWLALAASLLHVSLQGEFQRKLYKELVKNYNPLERPVANDSQP
LTVYFSLSLLQIMDVDEKNQVLTTNIWLQMSWTDHYLQWNVSEYPGVKTV
RFPDGQIWKPDILLYNSADERFDATFHTNVLVNSSGHCQYLPPGIFKSSC
YIDVRWFPFDVQHCKLKFGSWSYGGWSLDLQMQEADISGYIPNGEWDLVG
IPGKRSERFYECCKEPYPDVTFTVTMRRRMGYYLIQMYIPSLLIVILSWI
SFWINMDAAPARVGLGITTVLTMTTQSSGSRASLPKVSYVKAIDIWMAVC
LLFVFSALLEYAAVNFVSRQHKELLRFRRKRRHHKSPMLNLFQEDEAGEG
RFNFSAYGMGPACLQAKDGISVKGANNSNTTNPPPAPSKSPEEMRKLFIQ
RAKKIDKISRIGFPMAFLIFNMFYWIIYKIVRREDVHNQ.

In some embodiments, the chimeric LGIC receptor is a CHRNA7/GLRA1 (R228 junction) chimera, comprising the human α7-nAChR signal peptide (italics) and ligand binding domain (bold), fused to the human GlyRα1 ion pore domain (underlined):

(SEQ ID NO: 17)
MRCSPGGVWLALAASLLHVSLQGEFQRKLYKELVKNYNPLERPVANDSQ
PLTVYFSLSLLQIMDVDEKNQVLTTNIWLQMSWTDHYLQWNVSEYPGVK
TVRFPDGQIWKPDILLYNSADERFDATFHTNVLVNSSGHCQYLPPGIFK
SSCYIDVRWFPFDVQHCKLKFGSWSYGGWSLDLQMQEADISGYIPNGEW
DLVGIPGKRSERFYECCKEPYPDVTFTVTMRRQMGYYLIQMYIPSLLIV
ILSWISFWINMDAAPARVGLGITTVLTMTTQSSGSRASLPKVSYVKAID
IWMAVCLLFVFSALLEYAAVNFVSRQHKELLRFRRKRRHHKSPMLNLFQ
EDEAGEGRFNFSAYGMGPACLQAKDGISVKGANNSNTTNPPPAPSKSPE
EMRKLFIQRAKKIDKISRIGFPMAFLIFNMFYWIIYKIVRREDVHNQ.

In some embodiments, the chimeric LGIC receptor is a CHRNA7/GLRA1 (V224 junction) chimera, comprising the human α7-nAChR signal peptide (italics) and ligand binding domain (bold), fused to the human GlyRα1 ion pore domain (underlined):

(SEQ ID NO: 18)
MRCSPGGVWLALAASLLHVSLQGEFQRKLYKELVKNYNPLERPVANDSQP
LTVYFSLSLLQIMDVDEKNQVLTTNIWLQMSWTDHYLQWNVSEYPGVKTV
RFPDGQIWKPDILLYNSADERFDATFHTNVLVNSSGHCQYLPPGIFKSSC
YIDVRWFPFDVQHCKLKFGSWSYGGWSLDLQMQEADISGYIPNGEWDLVG
IPGKRSERFYECCKEPYPDVTFTVHLERQMGYYLIQMYIPSLLIVILSWI
SFWINMDAAPARVGLGITTVLTMTTQSSGSRASLPKVSYVKAIDIWMAVC
LLFVFSALLEYAAVNFVSRQHKELLRFRRKRRHHKSPMLNLFQEDEAGEG
RFNFSAYGMGPACLQAKDGISVKGANNSNTTNPPPAPSKSPEEMRKLFIQ
RAKKIDKISRIGFPMAFLIFNMFYWIIYKIVRREDVHNQ.

In some embodiments, the chimeric LGIC receptor is a CHRNA7/GLRA1 (Y233 junction) chimera, comprising the human α7-nAChR signal peptide (italics) and ligand binding domain (bold), fused to the human GlyRα1 ion pore domain (underlined):

(SEQ ID NO: 19)
MRCSPGGVWLALAASLLHVSLQGEFQRKLYKELVKNYNPLERPVANDSQP
LTVYFSLSLLQIMDVDEKNQVLTTNIWLQMSWTDHYLQWNVSEYPGVKTV
RFPDGQIWKPDILLYNSADERFDATFHTNVLVNSSGHCQYLPPGIFKSSC
YIDVRWFPFDVQHCKLKFGSWSYGGWSLDLQMQEADISGYIPNGEWDLVG
IPGKRSERFYECCKEPYPDVTFTVTMRRRTLYYLIQMYIPSLLIVILSWI
SFWINMDAAPARVGLGITTVLTMTTQSSGSRASLPKVSYVKAIDIWMAVC
LLFVFSALLEYAAVNFVSRQHKELLRFRRKRRHHKSPMLNLFQEDEAGEG
RFNFSAYGMGPACLQAKDGISVKGANNSNTTNPPPAPSKSPEEMRKLFIQ
RAKKIDKISRIGFPMAFLIFNMFYWIIYKIVRREDVHNQ.

In some embodiments, the chimeric LGIC receptor is a CHRNA7/GLRA1 chimera (R229 junction), comprising the human α7-nAChR signal peptide (italics) and ligand binding domain (bold), fused to the human GlyRα1 ion pore domain (underlined) comprising an α7-nAChR M2-M3 linker (lowercase):

(a)
(SEQ ID NO: 21, encoded by SEQ ID NO: 20)
MRCSPGGVWLALAASLLHVSLQGEFQRKLYKELVKNYNPLERPVANDSQP
LTVYFSLSLLQIMDVDEKNQVLTTNIWLQMSWTDHYLQWNVSEYPGVKTV
RFPDGQIWKPDILLYNSADERFDATFHTNVLVNSSGHCQYLPPGIFKSSC
YIDVRWFPFDVQHCKLKFGSWSYGGWSLDLQMQEADISGYIPNGEWDLVG
IPGKRSERFYECCKEPYPDVTFTVTMRRRMGYYLIQMYIPSLLIVILSWI
SFWINMDAAPARVGLGITTVLTMTTQSSGSeimpatsdsvSYVKAIDIWM
AVCLLFVFSALLEYAAVNFVSRQHKELLRFRRKRRHHKSPMLNLFQEDEA
GEGRFNFSAYGMGPACLQAKDGISVKGANNSNTTNPPPAPSKSPEEMRKL
FIQRAKKIDKISRIGFPMAFLIFNMFYWIIYKIVRREDVHNQ;
(b)
(SEQ ID NO: 23, encoded by SEQ ID NO: 22)
MRCSPGGVWLALAASLLHVSLQGEFQRKLYKELVKNYNPLERPVANDSQP
LTVYFSLSLLQIMDVDEKNQVLTTNIWLQMSWTDHYLQWNVSEYPGVKTV
RFPDGQIWKPDILLYNSADERFDATFHTNVLVNSSGHCQYLPPGIFKSSC
YIDVRWFPFDVQHCKLKFGSWSYGGWSLDLQMQEADISGYIPNGEWDLVG
IPGKRSERFYECCKEPYPDVTFTVTMRRRMGYYLIQMYIPSLLIVILSWI
SFWINMDAAPARVGLGITTVLTMTTQSSGSeimpatsdvpliaqAIDIWM
AVCLLFVFSALLEYAAVNFVSRQHKELLRFRRKRRHHKSPMLNLFQEDEA
GEGRFNFSAYGMGPACLQAKDGISVKGANNSNTTNPPPAPSKSPEEMRKL
FIQRAKKIDKISRIGFPMAFLIFNMFYWIIYKIVRREDVHNQ;
(c)
(SEQ ID NO: 25, encoded by SEQ ID NO: 24)
MRCSPGGVWLALAASLLHVSLQGEFQRKLYKELVKNYNPLERPVANDSQP
LTVYFSLSLLQIMDVDEKNQVLTTNIWLQMSWTDHYLQWNVSEYPGVKTV
RFPDGQIWKPDILLYNSADERFDATFHTNVLVNSSGHCQYLPPGIFKSSC
YIDVRWFPFDVQHCKLKFGSWSYGGWSLDLQMQEADISGYIPNGEWDLVG
IPGKRSERFYECCKEPYPDVTFTVTMRRRMGYYLIQMYIPSLLIVILSWI
SFWINMDAAPARVGLGITTVLTMTTQSSGSRASLPKVsdsvplIDIWMAV
CLLFVFSALLEYAAVNFVSRQHKELLRFRRKRRHHKSPMLNLFQEDEAGE
GRFNFSAYGMGPACLQAKDGISVKGANNSNTTNPPPAPSKSPEEMRKLFI
QRAKKIDKISRIGFPMAFLIFNMFYWIIYKIVRREDVHNQ;
(d)
(SEQ ID NO: 27, encoded by SEQ ID NO: 26)
MRCSPGGVWLALAASLLHVSLQGEFQRKLYKELVKNYNPLERPVANDSQP
LTVYFSLSLLQIMDVDEKNQVLTTNIWLQMSWTDHYLQWNVSEYPGVKTV
RFPDGQIWKPDILLYNSADERFDATFHTNVLVNSSGHCQYLPPGIFKSSC
YIDVRWFPFDVQHCKLKFGSWSYGGWSLDLQMQEADISGYIPNGEWDLVG
IPGKRSERFYECCKEPYPDVTFTVTMERQMGYYLIQMYIPSLLIVILSWI
SFWINMDAAPARVGLGITTVLTMTTQSSGSeimpatsdsvpliaqAIDIW
MAVCLLFVFSALLEYAAVNFVSRQHKELLRFRRKRRHHKSPMLNLFQEDE
AGEGRFNFSAYGMGPACLQAKDGISVKGANNSNTTNPPPAPSKSPEEMRK
LFIQRAKKIDKISRIGFPMAFLIFNMFYWIIYKIVRREDVHNQ;
(e)
(SEQ ID NO: 29, encoded by SEQ ID NO: 28)
MRCSPGGVWLALAASLLHVSLQGEFQRKLYKELVKNYNPLERPVANDSQP
LTVYFSLSLLQIMDVDEKNQVLTTNIWLQMSWTDHYLQWNVSEYPGVKTV
RFPDGQIWKPDILLYNSADERFDATFHTNVLVNSSGHCQYLPPGIFKSSC
YIDVRWFPFDVQHCKLKFGSWSYGGWSLDLQMQEADISGYIPNGEWDLVG
IPGKRSERFYECCKEPYPDVTFTVTMRRRTGYYLIQMYIPSLLIVILSWI
SFWINMDAAPARVGLGITTVLTMTTQSSGSeimnatsdsvDliaaAIDIW
MAVCLLFVFSALLEYAAVNFVSRQHKELLRFRRKRRHHKSPMLNLFQEDE
AGEGRFNFSAYGMGPACLQAKDGISVKGANNSNTTNPPPAPSKSPEEMRK
LFIQRAKKIDKISRIGFPMAFLIFNMFYWIIYKIVRREDVHNQ;
or
(f)
(SEQ ID NO: 31, encoded by SEQ ID NO: 30)
MRCSPGGVWLALAASLLHVSLQGEFQRKLYKELVKNYNPLERPVANDSQP
LTVYFSLSLLQIMDVDEKNQVLTTNIWLQMSWTDHYLQWNVSEYPGVKTV
RFPDGQIWKPDILLYNSADERFDATFHTNVLVNSSGHCQYLPPGIFKSSC
YIDVRWFPFDVQHCKLKFGSWSYGGWSLDLQMQEADISGYIPNGEWDLVG
IPGKRSERFYECCKEPYPDVTFTVTMRRRTLYYLIQMYIPSLLIVILSWI
SFWINMDAAPARVGLGITTVLTMTTQSSGSeimpatsdsvpliaqAIDIW
MAVCLLFVFSALLEYAAVNFVSRQHKELLRFRRKRRHHKSPMLNLFQEDE
AGEGRFNFSAYGMGPACLQAKDGISVKGANNSNTTNPPPAPSKSPEEMRK
LFIQRAKKIDKISRIGFPMAFLIFNMFYWIIYKIVRREDVHNQ.

In some embodiments, the chimeric LGIC receptor is a CHRNA7/GLRA1 chimera comprising the human α7-nAChR signal peptide (italics) and ligand binding domain (bold) comprising an GlyRα1 Cys-loop sequence (lowercase); fused to the human GlyRα1 ion pore domain (underlined). In some embodiments, the chimeric LGIC receptor comprises an amino acid sequence having a sequence identity of 80% or more, 85% or more, 90% or more, 95% or more, 97% or more, 98% or more, 99% or more, or 100%, to SEQ ID NO:33:

(SEQ ID NO: 33, encoded by SEQ ID NO: 32)
MRCSPGGVWLALAASLLHVSLQGEFQRKLYKELVKNYNPLERPVANDSQP
LTVYFSLSLLQIMDVDEKNQVLTTNIWLQMSWTDHYLQWNVSEYPGVKTV
RFPDGQIWKPDILLYNSADERFDATFHTNVLVNSSGHCQYLPPGIFKSSc
pmdlknfpmdvqtcKLKFGSWSYGGWSLDLQMQEADISGYIPNGEWDLVG
IPGKRSERFYECCKEPYPDVTFTVTMRRRMGYYLIQMYIPSLLIVILSWI
SFWINMDAAPARVGLGITTVLTMTTQSSGSRASLPKVSYVKAIDIWMAVC
LLFVFSALLEYAAVNFVSRQHKELLRFRRKRRHHKSPMLNLFQEDEAGEG
RFNFSAYGMGPACLQAKDGISVKGANNSNTTNPPPAPSKSPEEMRKLFIQ
RAKKIDKISRIGFPMAFLIFNMFYWIIYKIVRREDVHNQ.
(a)
(SEQ ID NO: 35, encoded by SEQ ID NO: 34)
MRCSPGGVWLALAASLLHVSLQGEFQRKLYKELVKNYNPLERPVANDSQP
LTVYFSLSLLQIMDVDEKNQVLTTNIWLQMSWTDHYLQWNVSEYPGVKTV
RFPDGQIWKPDILLYNSADERFDATFHTNVLVNSSGHCQYLPPGIFKSSc
pmdlknFPFDVQHCKLKFGSWSYGGWSLDLQMQEADISGYIPNGEWDLVG
IPGKRSERFYECCKEPYPDVTFTVTMRRRMGYYLIQMYIPSLLIVILSWI
SFWINMDAAPARVGLGITTVLTMTTQSSGSRASLPKVSYVKAIDIWMAVC
LLFVFSALLEYAAVNFVSRQHKELLRFRRKRRHHKSPMLNLFQEDEAGEG
RFNFSAYGMGPACLQAKDGISVKGANNSNTTNPPPAPSKSPEEMRKLFIQ
RAKKIDKISRIGFPMAFLIFNMFYWIIYKIVRREDVHNQ.

In some embodiments, the chimeric LGIC receptor is a CHRNA7/GLRA1 chimera comprising the human α7-nAChR signal peptide (italics) and ligand binding domain (bold) comprising an GlyRα1 β1-2 loop sequence (lowercase); fused to the human GlyRα1 ion pore domain (underlined):

(a)
(SEQ ID NO: 37, encoded by SEQ ID NO: 36)
MRCSPGGVWLALAASLLHVSLQGEFQRKLYKELVKNYNPLERPVANDSQP
LTVYFSLSLLQIMDVDettmVLTTNIWLQMSWTDHYLQWNVSEYPGVKTV
RFPDGQIWKPDILLYNSADERFDATFHTNVLVNSSGHCQYLPPGIFKSSC
YIDVRWFPFDVQHCKLKFGSWSYGGWSLDLQMQEADISGYIPNGEWDLVG
IPGKRSERFYECCKEPYPDVTFTVTMRRRMGYYLIQMYIPSLLIVILSWI
SFWINMDAAPARVGLGITTVLTMTTQSSGSRASLPKVSYVKAIDIWMAVC
LLFVFSALLEYAAVNFVSRQHKELLRFRRKRRHHKSPMLNLFQEDEAGEG
RFNFSAYGMGPACLQAKDGISVKGANNSNTTNPPPAPSKSPEEMRKLFIQ
RAKKIDKISRIGFPMAFLIFNMFYWIIYKIVRREDVHNQ.

In some embodiments, the chimeric LGIC receptor is a CHRNA7/GLRA1 chimera comprising the human α7-nAChR signal peptide (italics) and ligand binding domain (bold) comprising an GlyRα1 β1-2 loop sequence (lowercase) and Cys-loop sequence (lowercase); fused to the human GlyRα1 ion pore domain (underlined):

(a)
(SEQ ID NO: 39, encoded by SEQ ID NO: 38)
MRCSPGGVWLALAASLLHVSLQGEFQRKLYKELVKNYNPLERPVANDSQP
LTVYFSLSLLQIMDiaettmdLTTNIWLQMSWTDHYLQWNVSEYPGVKTV
RFPDGQIWKPDILLYNSADERFDATFHTNVLVNSSGHCQYLPPGIFKSSc
pmdlknfpmdvqtcKLKFGSWSYGGWSLDLQMQEADISGYIPNGEWDLVG
IPGKRSERFYECCKEPYPDVTFTVTMRRRMGYYLIQMYIPSLLIVILSWI
SFWINMDAAPARVGLGITTVLTMTTQSSGSRASLPKVSYVKAIDIWMAVC
LLFVFSALLEYAAVNFVSRQHKELLRFRRKRRHHKSPMLNLFQEDEAGEG
RFNFSAYGMGPACLQAKDGISVKGANNSNTTNPPPAPSKSPEEMRKLFIQ
RAKKIDKISRIGFPMAFLIFNMFYWIIYKIVRREDVHNQ.
(b)
(SEQ ID NO: 41, encoded by SEQ ID NO: 40)
MRCSPGGVWLALAASLLHVSLQGEFQRKLYKELVKNYNPLERPVANDSQP
LTVYFSLSLLQIMDVDettmVLTTNIWLQMSWTDHYLQWNVSEYPGVKTV
RFPDGQIWKPDILLYNSADERFDATFHTNVLVNSSGHCQYLPPGIFKSSc
pmdlknfpmdvqtcKLKFGSWSYGGWSLDLQMQEADISGYIPNGEWDLVG
IPGKRSERFYECCKEPYPDVTFTVTMRRRMGYYLIQMYIPSLLIVILSWI
SFWINMDAAPARVGLGITTVLTMTTQSSGSRASLPKVSYVKAIDIWMAVC
LLFVFSALLEYAAVNFVSRQHKELLRFRRKRRHHKSPMLNLFQEDEAGEG
RFNFSAYGMGPACLQAKDGISVKGANNSNTTNPPPAPSKSPEEMRKLFIQ
RAKKIDKISRIGFPMAFLIFNMFYWIIYKIVRREDVHNQ.
(c)
(SEQ ID NO: 43, encoded by SEQ ID NO: 42)
MRCSPGGVWLALAASLLHVSLQGEFQRKLYKELVKNYNPLERPVANDSQP
LTVYFSLSLLQIMDVDettmVLTTNIWLQMSWTDHYLQWNVSEYPGVKTV
RFPDGQIWKPDILLYNSADERFDATFHTNVLVNSSGHCQYLPPGIFKSSc
pmdlknfpmdvqtcKLKFGSWSYGGWSLDLQMQEADISGYIPNGEWDLVG
IPGKRSERFYECCKEPYPDVTFTVTMERQMGYYLIQMYIPSLLIVILSWI
SFWINMDAAPARVGLGITTVLTMTTQSSGSRASLPKVSYVKAIDIWMAVC
LLFVFSALLEYAAVNFVSRQHKELLRFRRKRRHHKSPMLNLFQEDEAGEG
RFNFSAYGMGPACLQAKDGISVKGANNSNTTNPPPAPSKSPEEMRKLFIQ
RAKKIDKISRIGFPMAFLIFNMFYWIIYKIVRREDVHNQ.
(d)
(SEQ ID NO: 45, encoded by SEQ ID NO: 44)
MRCSPGGVWLALAASLLHVSLQGEFQRKLYKELVKNYNPLERPVANDSQP
LTVYFSLSLLQIMDiaettmdLTTNIWLQMSWTDHYLQWNVSEYPGVKTV
RFPDGQIWKPDILLYNSADERFDATFHTNVLVNSSGHCQYLPPGIFKSSc
pmdlknfpmdvqtcKLKFGSWSYGGWSLDLQMQEADISGYIPNGEWDLVG
IPGKRSERFYECCKEPYPDVTFTVTMERQMGYYLIQMYIPSLLIVILSWI
SFWINMDAAPARVGLGITTVLTMTTQSSGSRASLPKVSYVKAIDIWMAVC
LLFVFSALLEYAAVNFVSRQHKELLRFRRKRRHHKSPMLNLFQEDEAGEG
RFNFSAYGMGPACLQAKDGISVKGANNSNTTNPPPAPSKSPEEMRKLFIQ
RAKKIDKISRIGFPMAFLIFNMFYWIIYKIVRREDVHNQ.

In some embodiments, the chimeric LGIC receptor is a CHRNA7/GLRA1 chimera comprising the human α7-nAChR signal peptide (italics) and ligand binding domain (bold) comprising an GlyRα1 β1-2 loop sequence (lowercase); fused to the human GlyRα1 ion pore domain (underlined) comprising human α7-nAChR M2-M3 linker (lowercase):

(a)
(SEQ ID NO: 47, encoded by SEQ ID NO: 46)
MRCSPGGVWLALAASLLHVSLQGEFQRKLYKELVKNYNPLERPVANDSQP
LTVYFSLSLLQIMDVDettmVLTTNIWLQMSWTDHYLQWNVSEYPGVKTV
RFPDGQIWKPDILLYNSADERFDATFHTNVLVNSSGHCQYLPPGIFKSSC
YIDVRWFPFDVQHCKLKFGSWSYGGWSLDLQMQEADISGYIPNGEWDLVG
IPGKRSERFYECCKEPYPDVTFTVTMRRRMGYYLIQMYIPSLLIVILSWI
SFWINMDAAPARVGLGITTVLTMTTQSSGSeimpatsdsvpliaqAIDIW
MAVCLLFVFSALLEYAAVNFVSRQHKELLRFRRKRRHHKSPMLNLFQEDE
AGEGRFNFSAYGMGPACLQAKDGISVKGANNSNTTNPPPAPSKSPEEMRK
LFIQRAKKIDKISRIGFPMAFLIFNMFYWIIYKIVRREDVHNQ.

In some embodiments, the chimeric LGIC receptor is a CHRNA7/GLRA1 chimera comprising the human α7-nAChR signal peptide (italics) and ligand binding domain (bold) comprising a GlyRα1 Cys-loop sequence (lowercase); fused to the human GlyRα1 ion pore domain (underlined) comprising a human α7-nAChR M2-M3 linker (lowercase):

(a)
(SEQ ID NO: 49, encoded by SEQ ID NO: 48)
MRCSPGGVWLALAASLLHVSLQGEFQRKLYKELVKNYNPLERPVANDSQP
LTVYFSLSLLQIMDVDEKNQVLTTNIWLQMSWTDHYLQWNVSEYPGVKTV
RFPDGQIWKPDILLYNSADERFDATFHTNVLVNSSGHCQYLPPGIFKSSc
pmdlknfpmdvqtcKLKFGSWSYGGWSLDLQMQEADISGYIPNGEWDLVG
IPGKRSERFYECCKEPYPDVTFTVTMRRRMGYYLIQMYIPSLLIVILSWI
SFWINMDAAPARVGLGITTVLTMTTQSSGSeimDatsdsvDliaqAIDIW
MAVCLLFVFSALLEYAAVNFVSRQHKELLRFRRKRRHHKSPMLNLFQEDE
AGEGRFNFSAYGMGPACLQAKDGISVKGANNSNTTNPPPAPSKSPEEMRK
LFIQRAKKIDKISRIGFPMAFLIFNMFYWIIYKIVRREDVHNQ.

In some embodiments, the chimeric LGIC receptor is a HTR3A/GLRA1 chimera (R241 junction), comprising the human 5HT3A serotonin receptor signal peptide (italics) and ligand binding domain (bold), fused to the human GlyRα1 ion pore domain (underlined):

(a)
(SEQ ID NO: 50)
MLLWVQQALLALLLPTLLAQGEARRSRNTTRPALLRLSDYLLTNYRKGVR
PVRDWRKPTTVSIDVIVYAILNVDEKNQVLTTYIWYRQYWTDEFLQWNPE
DFDNITKLSIPTDSIWVPDILINEFVDVGKSPNIPYVYIRHQGEVQNYKP
LQVVTACSLDIYNFPFDVQNCSLTFTSWLHTIQDINISLWRLPEKVKSDR
SVFMNQGEWELLGVLPYFREFSMESSNYYAEMKFYVVIRRRMGYYLIQMY
IPSLLIVILSWISFWINMDAAPARVGLGITTVLTMTTQSSGSRASLPKVS
YVKAIDIWMAVCLLFVFSALLEYAAVNFVSRQHKELLRFRRKRRHHKSPM
LNLFQEDEAGEGRFNFSAYGMGPACLQAKDGISVKGANNSNTTNPPPAPS
KSPEEMRKLFIQRAKKIDKISRIGFPMAFLIFNMFYWIIYKIVRREDVHN
Q.

In some embodiments, the chimeric LGIC receptor is a HTR3A/GLRA1 chimera (V236 junction) comprising the human 5HT3A serotonin receptor signal peptide (italics) and ligand binding domain (bold), fused to the human GlyRα1 ion pore domain (underlined):

(a)
(SEQ ID NO: 51)
MLLWVQQALLALLLPTLLAQGEARRSRNTTRPALLRLSDYLLTNYRKGVR
PVRDWRKPTTVSIDVIVYAILNVDEKNQVLTTYIWYRQYWTDEFLQWNPE
DFDNITKLSIPTDSIWVPDILINEFVDVGKSPNIPYVYIRHQGEVQNYKP
LQVVTACSLDIYNFPFDVQNCSLTFTSWLHTIQDINISLWRLPEKVKSDR
SVFMNQGEWELLGVLPYFREFSMESSNYYAEMKFYVHLERQMGYYLIQMY
IPSLLIVILSWISFWINMDAAPARVGLGITTVLTMTTQSSGSRASLPKVS
YVKAIDIWMAVCLLFVFSALLEYAAVNFVSRQHKELLRFRRKRRHHKSPM
LNLFQEDEAGEGRFNFSAYGMGPACLQAKDGISVKGANNSNTTNPPPAPS
KSPEEMRKLFIQRAKKIDKISRIGFPMAFLIFNMFYWIIYKIVRREDVHN
Q.

In some embodiments, the chimeric LGIC receptor is a GABRB3/GLRA1 chimera (Y245 junction), comprising the human GABA-A β3 signal peptide (italics) and ligand binding domain (bold), fused to the human GlyRα1 ion pore domain (underlined):

(a)
(SEQ ID NO: 52)
MWGLAGGRLFGIFSAPVLVAVVCCAQSVNDPGNMSFVKETVDKLLKGYDI
RLRPDFGGPPVCVGMNIDIASIDMVSEVNMDYTLTMYFQQYWRDKRLAYS
GIPLNLTLDNRVADQLWVPDTYFLNDKKSFVHGVTVKNRMIRLHPDGTVL
YGLRITTTAACMMDLRRYPLDEQNCTLEIESYGYTTDDIEFYWRGGDKAV
TGVERIELPQFSIVEHRLVSRNVVFATGAYPRLSLSFRLKRNIGYMGYYL
IQMYIPSLLIVILSWISFWINMDAAPARVGLGITTVLTMTTQSSGSRASL
PKVSYVKAIDIWMAVCLLFVFSALLEYAAVNFVSRQHKELLRFRRKRRHH
KSPMLNLFQEDEAGEGRFNFSAYGMGPACLQAKDGISVKGANNSNTTNPP
PAPSKSPEEMRKLFIQRAKKIDKISRIGFPMAFLIFNMFYWIIYKIVRRE
DVHNQ.

As discussed above, in some aspects, the subject engineered receptor comprises at least one amino acid mutation that alters the potency of a ligand on the engineered receptor relative to its potency on the unmutated parental receptor. Put another way, the one or more amino acid mutations, e.g. a loss-of-function mutations or a gain-of-function mutations, shift the responsiveness of the engineered receptor to the ligand relative to the responsiveness of the unmutated parental receptor. In some such embodiments the one or more mutations is in the ligand binding domain of the engineered receptor. In some embodiments, as when the ligand binding domain of the engineered receptor is a Cys-loop receptor protein, the one or more amino acid mutations is a substitution at a residue corresponding to a residue of α7-nAChR (SEQ ID NO:4) selected from the group consisting of W77, Y94, R101, W108, Y115, T128, N129, V130, L131, Q139, L141, Y151, 5170, W171, 5172, 5188, Y190, Y210, C212, C213 and Y217. In some embodiments, one residue is substituted. In some embodiments, 2, 3, 4, or 5 or more residues are substituted, e.g. 6, 7, 8, 9 or 10 residues are substituted. In certain embodiments, the residue corresponds to a residue of α7-nAChR (SEQ ID NO:4) that is selected from the group consisting of W77, R101, Y115, N129, L131, S170, S172, and S188. In certain embodiments, the one or more substitutions is within an α7-nAChR sequence.

In some embodiments, the one or more substitutions decreases, e.g. 2-fold or more, 3-fold or more, 4-fold or more. 5-fold or more, 10-fold or more, 20-fold or more, 30-fold or more, 50-fold or more, or 100-fold, the responsiveness of an engineered receptor to acetylcholine and a non-native ligand. In certain embodiments, the one or more substitutions is a substitution corresponding to R101I, R101S, R101D, Y115L, Y115M, Y115D, Y115T, T128M, T128R, T1281, N1291, N129V, N129P, N129W, N129T, N129D, N129E, L131E, L131P, L131T, L131D, L131S, L141S, L141R, W171F, W171H, S172F, S172Y, S172R, S172D, C212A, C212L, or C213P of α7-nAChR. In other instances, the one or more substitutions decreases the potency of acetylcholine on the engineered receptor selectively. In other words, the one or more substitutions decreases the responsiveness of the engineered receptor to acetylcholine while essentially maintaining responsiveness to non-native ligand or otherwise decreasing the responsiveness of the engineered receptor to acetylcholine 2-fold or more, e.g. 3-fold, 4-fold, 5-fold or more, in some instances 10-fold, 20-fold, 50-fold, or 100-fold or more, than it decreases the responsiveness of the engineered receptor to non-native ligand. Exemplary substitutions, namely, a substitution corresponding to L131E, L131S, L131T, L131D, or S172D of α7-nAChR. In yet other embodiments, the one or more substitutions decreases the potency of a non-native ligand on the engineered receptor selectively. In other words, the one or more substitutions decreases the responsiveness of the engineered receptor to non-native ligand while essentially maintaining responsiveness to acetylcholine or otherwise decreasing the responsiveness of the engineered receptor to non-native ligand 2-fold or more, e.g. 3-fold, 5-fold or more, in some instances 10-fold, 20-fold or 50-fold or more, than it decreases the responsiveness of the engineered receptor to acetylcholine. Exemplary substitutions include a substitution corresponding to W77M, Y115W, S172T, or S172C of α7-nAChR. In certain embodiments, the one or more substitutions is within an α7-nAChR sequence. In certain embodiments, the non-native ligand is selected from AZD-0328, TC6987, ABT-126 and Facinicline/RG3487.

In other embodiments, the one or more substitutions increases, e.g. 2-fold or more, 3-fold or more, 4-fold or more. 5-fold or more, 10-fold or more, 20-fold or more, 30-fold or more, 50-fold or more, or 100-fold, the responsiveness of the engineered receptor to acetylcholine and/or non-native ligand. Exemplary substitutions include a substitution corresponding to L131N, L141W, S170G, S170A, S170L, S170I, S170V, S170P, S170F, S170M, S170T, S170C, S172T, S172C, 51881, S188V, S188F, S188M, S188Q, S188T, S188P or S188W. In some instance, the one or more substitutions increases potency of both acetylcholine and non-native ligand, e.g. substitutions corresponding to L131N, S170G, S170A, S170L, S170I, S170V, S170P, S170F, S170M, S170T, S170C, S172T, S1881, S188V, S188F, S188M, S188Q and S188T of α7-nAChR. In other instances, the one or more substitutions increases the potency of acetylcholine on the engineered receptor selectively. In other words, the one or more substitutions increases the responsiveness of the engineered receptor to acetylcholine 2-fold or more, e.g. 3-fold, 4-fold, or 5-fold or more, in some instances 10-fold, 20-fold, 50-fold, or 100-fold, than it increases the responsiveness of the engineered receptor to non-native ligand, e.g. substitutions corresponding to L141W, S172T, S172C, S188P or S188W, of α7-nAChR. In certain embodiments, the one or more substitutions is within an α7-nAChR sequence. In certain embodiments, the non-native ligand is selected from AZD-0328, TC6987, ABT-126 and Facinicline/RG3487. In yet other instances, the one or more substitutions increases the potency of the non-native ligand on the engineered receptor selectively. In other words, the one or more substitutions increases the responsiveness of the engineered receptor to non-native ligand 2-fold or more, e.g. 3-fold, 5-fold or more, in some instances 10-fold, 20-fold or 50-fold or more, than it increases the responsiveness of the engineered receptor to acetylcholine.

In some embodiments, the amino acid residue that is mutated in the subject engineered receptor is not an amino acid corresponding to R27, E41, Q79, Q139, L141, G175, Y210, P216, Y217, or D219 of wild type α7 nAChR (SEQ ID NO:4). In some embodiments, the amino acid residue that is mutated in the subject engineered receptor is an amino acid corresponding to R27, E41, Q79, Q139, L141, G175, Y210, P216, Y217, or D219 of wild type α7 nAChR (SEQ ID NO:4). In some embodiments, the substitution is not a substitution corresponding to W77F, W77Y, W77M, Q79A, Q79Q, Q79S, Q79G, Y115F, L131A, L131G, L131M, L131N, L131Q, L131V, L131F, Q139G, Q139L, G175K, G175A, G175F, G175H, G175M, G175R, G175S, G175V, Y210F, P2161, Y217F, or D219A in wild type α7 nAChR. In some embodiments, the substitution is a substitution corresponding to W77F, W77Y, W77M, Q79A, Q79Q, Q79S, Q79G, Y115F, L131A, L131G, L131M, L131N, L131Q, L131V, L131F, Q139G, Q139L, G175K, G175A, G175F, G175H, G175M, G175R, G175S, G175V, Y210F, P2161, Y217F, or D219A in wild type α7 nAChR. In some embodiments, when such a substitution exists within the engineered receptor, it exists in combination with one or more of the amino acid mutations described herein.

For example, it has been discovered that residues Y94, Y115, Y151, and Y190 of α7-nAChR (SEQ ID NO:4) mediate binding of the native ligand acetylcholine. Mutations at these residues will reduce binding of acetylcholine and hence are loss of function mutations. In contrast, residues W77, Y115, N129, V130, L131, Q139, L141, 5170, Y210, C212, C213 and Y217 of the α7-nAChR mediate the binding of non-native ligand AZD0328 to this receptor, and mutation of these residues may increase the affinity of AZD0328 and/or other ligands for this receptor and hence be gain-of-function mutations. In some embodiments, the subject engineered receptor comprises a mutation in one or more amino acid residues of the ligand binding domain region of α7-nAChR (SEQ ID NO:4) or the ligand binding domain of a chimeric receptor that comprises the ligand binding domain region of α7-nAChR, wherein the one or more amino acid residues is selected from the group consisting of W77, Y94, Y115, N129, V130, L131, Q139, L141, Y151, 5170, Y190, Y210, C212, C213 and Y217. In certain embodiments, the mutation in the one or more amino acid residues of the ligand binding domain region of α7-nAChR (SEQ ID NO:4) or the ligand binding domain of a chimeric receptor that comprises the ligand binding domain region of α7-nAChR is a substitution at one or more amino acid residues selected from the group consisting of W77, Y94, Y115, N129, V130, L131, Q139, L141, Y151, 5170, Y190, Y210, C212, C213 and Y217.

As another example, it has been discovered that residues Y115, L131, L141, S170, W171, S172, C212, and Y217 of α7-nAChR (SEQ ID NO:4) mediate binding of acetylcholine and/or nicotine, and mutations at one or more of these residues will reduce binding of acetylcholine and/or nicotine. R101, Y115, L131, L141, W171, 5172, 5188, Y210, and Y217 of α7-nAChR mediate binding of the non-native ligand ABT126, and mutation of one or more of these residues is expected to increase the affinity of ABT126 and/or other ligands for α7-nAChR. R101, Y115, T128, N129, L131, L141, W171, 5172, Y210, C212, C213 and Y217 of α7-nAChR mediate binding of the non-native ligand TC6987, and mutation of one or more of these residues is expected to increase the affinity of TC6987 and/or other ligands for α7-nAChR. R101, N120, L131, L141, 5170, W171, 5172, Y210, and Y217 of α7-nAChR mediate binding of the non-native ligand Facinicline/RG3487, and mutation of one or more of these residues is expected to increase the affinity of Facinicline/RG3487 and/or other ligands for α7-nAChR. In some embodiments, the subject engineered receptor comprises a mutation in one or more amino acid residues of the ligand binding domain region of α7-nAChR or the ligand binding domain of a chimeric receptor that comprises the ligand binding domain region of α7-nAChR, where the one or more amino acid residues is selected from the group consisting of R101, Y115, T128, N120, N129, L131, L141, 5170, W171, 5172, 5188, Y210, C212, C213 and Y217. In some embodiments, the one or more amino acid residues alters the binding of acetylcholine and/or nicotine to α7-nAChR, wherein the amino acid is selected from the group consisting of Y115, L131, L141, S170, W171, S172, C212 and Y217 of α7-nAChR. In certain such embodiments, the amino acid is selected from C212 and S170. In some embodiments, the mutation in the one or more amino acid residues alters the binding of ABT126 to α7-nAChR, wherein one or more amino acid residues is selected from the group consisting of R101, Y115, L131, L141, W171, 5172, 5188, Y210, and Y217 of α7-nAChR. In certain such embodiments, the amino acid is selected from R101, 5188, and Y210. In some embodiments, the mutation in the one or more amino acid residues alters the binding of TC6987 to α7-nAChR, wherein one or more amino acid residues is selected from the group consisting of R101, Y115, T128, N129, L131, L141, W171, 5172, Y210, C212, C213 and Y217 of α7-nAChR. In certain such embodiments, the amino acid is selected from R101, T128, N129, Y210 and C213. In some embodiments, the mutation in the one or more amino acid residues alters the binding of Facinicline/RG3487 to α7-nAChR, wherein one or more amino acid residues is selected from the group consisting R101, N120, L131, L141, 5170, W171, 5172, Y210, and Y217 of α7-nAChR. In certain such embodiments, the amino acid is selected from Y210, R101, and N129.

As another example, it has been discovered that residues W85, R87, Y136, Y138, G146, N147, Y148, K149, S177, S178, L179, Y228, and Y229 of 5HT3 (SEQ ID NO:6) mediate binding of serotonin, and mutations at one or more of these residues will reduce binding of serotonin to 5HT3. D64, I66, W85, R87, Y89, N123, G146, Y148, T176, 5177, S178, W190, R191, F221, E224, Y228, Y229 and E231 of 5HT3 mediate binding of the non-native ligand Cilansetron, and mutation of one or more of these residues is expected to increase the affinity of Cilansetron and/or other ligands for 5HT3. In some embodiments, the subject engineered receptor comprises a mutation in one or more amino acid residues of the ligand binding domain region 5HT3A or the ligand binding domain of a chimeric receptor that comprises the ligand binding domain region of 5HT3, where the one or more amino acid residues is selected from the group consisting of D64, I66, W85, R87, Y89, N123, Y136, Y138, G146, N147, Y148, K149, T176, S177, S178, L179, W190, R191, F221, E224, Y228, Y229, and E231. In some embodiments, the mutation in the one or more amino acid residues alters the binding of serotonin to 5HT3, wherein the amino acid is selected from the group consisting of W85, R87, Y136, Y138, G146, N147, Y148, K149, S177, S178, L179, Y228, and Y229 of 5HT3A. In certain such embodiments, the amino acid is selected from Y136, Y138, N147, K149, and L179. In some embodiments, the mutation in the one or more amino acid residues alters the binding of Cilansetron to 5HT3 wherein one or more amino acid residues is selected from the group consisting of D64, I66, W85, R87, Y89, N123, G146, Y148, T176, S177, S178, W190, R191, F221, E224, Y228, Y229 and E231 of 5HT3A. In certain such embodiments, the amino acid is selected from D64, I66, Y89, N123, T176, W190, R191, F221, E224, and E231.

In some embodiment, the one or more mutations that affects the ability of a ligand to modulate the activity of the LGIC is located in the ion pore domain of the LGIC. For example, residue T279 of the serotonin receptor 5HT3A mediates the way in which the ligand modulates the activity of the channel, such that mutation of this residue to, e.g. serine (T279S), converts the effect from being antagonistic (i.e., reducing the activity of the LGIC) to agonistic (i.e. promoting the activity of the channel). In some embodiments, the subject ligand gated ion channel comprises a mutation in one or more amino acid residues of the ion pore domain of the human 5HT3A (SEQ ID NO:6) or the ion pore domain of a chimeric LGIC receptor that comprises the ion pore domain of 5HT3A, where the substitution is in an amino acid corresponding to 279 of SEQ ID NO:6. In certain embodiments, the substitution is a T279S substitution relative to SEQ ID NO:6.

The disclosure provides engineered receptors having two or more mutations, such as amino acid substitutions, as compared to the parental receptor. In some embodiments, the parental receptor is a chimeric receptor. In some embodiments, the parental receptor comprises an amino acid sequence of SEQ ID NO: 33. In some embodiments, the engineered receptors comprise two amino acid substitutions as compared to the parental receptor comprising an amino acid sequence of SEQ ID NO: 33.

In some embodiments, the two amino acid substitutions are at a pair of amino acid residues selected from the group consisting of L131 and S172, Y115 and S170, and Y115 and L131. In some embodiments, the ligand binding domain comprises two amino acid substitutions at a pair of amino acids residues selected from the group consisting of L131 and S172, Y115 and S170, and Y115 and L131. In some embodiments, the ligand binding domain comprises a pair of amino acid substitutions selected from the group consisting of L131S and S172D, L131T and S172D, L131D and S172D, Y115D and S170T, Y115D and L131Q, and Y115D and L131E. In some embodiments, the ligand binding domain comprises an amino acid substitution of L131E.

In some embodiments, the potency of the engineered receptor to acetylcholine is lower than the potency of the human α7 nicotinic acetylcholine receptor (α7-nAChR) to acetylcholine. In some embodiments, the potency of the engineered receptor to acetylcholine is at least about 1.5-fold (for example, about 2-fold lower, about 3-fold, about 4-fold, about 5-fold, about 6-fold, about 7-fold, about 8-fold, about 9-fold, about 10-fold, about 12-fold, about 15-fold, about 20-fold, about 30-fold, about 40-fold, about 50-fold, about 60-fold, about 70-fold, about 80-fold, about 90-fold, or about 100-fold, including all subranges and values that lie therebetween) lower than the potency of the human α7 nicotinic acetylcholine receptor (α7-nAChR) to acetylcholine.

In some embodiments, the potency of the engineered receptor to a non-native ligand is about the same as the potency of the human α7 nicotinic acetylcholine receptor (α7-nAChR) to the non-native ligand. In some embodiments, the potency of the engineered receptor to a non-native ligand is higher than the potency of the human α7 nicotinic acetylcholine receptor (α7-nAChR) to the non-native ligand. In some embodiments, the potency of the engineered receptor to the non-native ligand is at least about 1.5-fold (for example, about 2-fold lower, about 3-fold, about 4-fold, about 5-fold, about 6-fold, about 7-fold, about 8-fold, about 9-fold, about 10-fold, about 12-fold, about 15-fold, about 20-fold, about 30-fold, about 40-fold, about 50-fold, about 60-fold, about 70-fold, about 80-fold, about 90-fold, or about 100-fold, including all subranges and values that lie therebetween) higher than the potency of the human α7 nicotinic acetylcholine receptor (α7-nAChR) to the non-native ligand. In some embodiments, determining the potency comprises determining the EC50.

In some embodiments, the efficacy of the engineered receptor in the presence of a non-native ligand is higher than the efficacy the human α7 nicotinic acetylcholine receptor (α7-nAChR) in presence of the non-native ligand. In some embodiments, the efficacy of the engineered receptor in the presence of a non-native ligand is at least about 1.5-fold (for example, about 2-fold lower, about 3-fold, about 4-fold, about 5-fold, about 6-fold, about 7-fold, about 8-fold, about 9-fold, about 10-fold, about 12-fold, about 15-fold, about 20-fold, about 30-fold, about 40-fold, about 50-fold, about 60-fold, about 70-fold, about 80-fold, about 90-fold, or about 100-fold, including all subranges and values that lie therebetween) higher than the efficacy the human α7 nicotinic acetylcholine receptor (α7-nAChR) in presence of the non-native ligand. In some embodiments, determining the efficacy comprises determining the amount of current passed through the engineered receptor in vitro in the presence of the non-native ligand.

In some aspects, the subject ligand-gated ion channel comprises one or more non-desensitizing mutations. When used in the context of a ligand-gated ion channel, “desensitization” refers to the progressive reduction in ionic flux in the prolonged presence of agonist. This results in a progressive loss of responsiveness of the neuron to the ligand. By a non-desensitizing mutation, it is meant an amino acid mutation that prevents the LGIC from becoming desensitized to ligand, thereby preventing the neuron from becoming less responsive or nonresponsive to ligand. Non-desensitizing mutations can be readily identified by introducing the LGIC carrying the mutation into a neuron and analyzing the current flux over time during prolonged exposure to ligand. If the LGIC does not comprise a non-desensitizing mutation, the current will restore from peak to steady state during prolonged exposure, whereas if the LGIC comprises a non-desensitizing mutation, the current will remain at peak flux for the duration of exposure to ligand. Exemplary amino acid mutations that result in desensitization include a V322L mutation in the human GlyRα1 (V294L post-processing of the pro-protein to remove the signal peptide) and a L321V mutation in human GABA-A receptor GABRB3 (L296V post-processing of the pro-protein to remove the signal peptide). In some embodiments, the desensitizing mutation is the replacement of amino acid residues at or near the C-terminus of the LGIC with a desensitizing sequence, for example, a sequence having 90% identity or more to IDRLSRIAFPLLFGIFNLVYWATYLNREPQL (SEQ ID NO:53) derived from the C terminus of the protein encoded by GABAR1, e.g. the replacement of residues 455-479 in GABRR1 with IDRLSRIAFPLLFGIFNLVYWATYLNREPQL (SEQ ID NO:53). LGIC desensitization, methods for measuring desensitization of LGICs, and mutations that are non-desensitizing are well known in the art; see, e.g. Gielen et al. Nat Commun 2015 Apr. 20, 6:6829, and Keramidas et al. Cell Mol Life Sci. 2013 April; 70(7):1241-53, the full disclosures of which are incorporated herein by reference.

In some aspects, the subject ligand-gated ion channel comprises one or more conversion mutations. By a conversion mutation, it is meant a mutation that changes the permeability of the ion pore domain of the LGIC such that it becomes permissive to the conductance of a non-native ion, i.e. an ion that does not naturally allow to pass through. In some cases, the mutation converts the permeability from cation to anion, for example the replacement of amino acid residues 260-281 in human α7-nAChR (CHRNA7) (EKISLGITVLLSLTVFMLLVAE, SEQ ID NO:54) or the corresponding amino acids in another cation-permeable LGIC with the peptide sequence PAKIGLGITVLLSLTTFMSGVAN (SEQ ID NO:55). In some cases, the mutation converts the permeability from anion to cation, for example, the substitution of amino acid residue 279 of GLRA1 or the corresponding amino acid in another anion-permeable LGIC to glutamic acid (E), (which, as an A293E substitution in GLRA1 converts the LGIC from being anion-permissive to calcium-permissive), or the deletion of amino acid residue 278 of GLRA1 or the corresponding amino acid in another anion-permeable LGIC, the substitution of amino acid residue 279 of GLRA1 or the corresponding amino acid in another anion-permeable LGIC to glutamic acid (E), and the substitution of amino acid residue 293 of GLRA1 or the corresponding amino acid in another anion-permeable LGIC to valine (V) (which, as a P2784, A279E, T293V in GLRA1 converts the LGIC from being anion-permissive to cation-permissive).

Additional engineered receptors beyond those described herein can be readily identified by in vitro screening and validation methods. In some embodiments, a library of parental receptor mutants is generated from a limited number of parental receptors. The parental receptors can be mutated using methods known in the art, including error prone PCR.

In some embodiments, the library of parental receptor mutants is then transfected into yeast or mammalian cells and screened in high throughput to identify functional receptors (e.g., to identify parental receptor mutants that are capable of signaling in response to a binding agent or ligand). In some embodiments, the functional parental receptor mutants identified in this primary screen is then expressed in mammalian cells and screened for responsiveness to binding agents or ligands, e.g. by the plate reader and/or electrophysiology assays described herein. The parental receptor mutants that demonstrate either increased binding affinity for agonist binding agents, or that enable the use of antagonist or modulator binding agents as agonists in the secondary screen can then be selected and carried though further in vitro and/or in vivo validation and characterization assays. Such screening assays are known in the art, for example Armbruster, B. N. et al. (2007) PNAS, 104, 5163-5168; Nichols, C. D. and Roth, B. L. (2009) Front. Mol. Neurosci. 2, 16; Dong, S. et al. (2010) Nat. Protoc. 5, 561-573; Alexander, G. M. et al. (2009) Neuron 63, 27-39; Guettier, J. M. et al. (2009) PNAS 106, 19197-19202; Ellefson J. W. et al. (2014) Nat Biotechnol, 32(1):97-101; Maranhao A C and Ellington A D. (2017) ACS Synth Biol. 20; 6(1):108-119; Talwar S et al. (2013) PLoS One; 8(3):e58479; Gilbert D. F. et al. (2009) Front Mol Neurosci. 30; 2:17; Lynagh and Lynch, (2010), Biol Chem. 14:285(20), 14890-14897; Islam R. et al. (2016) ACS Chem Neurosci. 21; 7(12):1647-1657; and Myers et al. (2008) Neuron. 8:58(3): 362-373.

### D. Binding Agents

The terms “binding agent” or “agent” are used interchangeably herein and refer to exogenous drugs or compounds with a known mechanism of action on a mammalian cell (e.g., are known to act as an agonist, antagonist, or modulator of a receptor). Binding agents can include proteins, lipids, nucleic acids, and/or small molecules. In some embodiments, binding agents include drugs or compounds that have been approved by the US Food and Drug Administration (FDA) for clinical use in the treatment of a particular disease (e.g., a neurological disease). In some embodiments, binding agents include drugs or compounds that have not been approved by the FDA for clinical use, but have been tested in one or more clinical trials, are currently being tested in one or more clinical trials, and/or are anticipated to be tested in one or more clinical trials. In some embodiments, binding agents include drugs or compounds that have not been approved by the FDA for clinical use, but are routinely used in laboratory research. In some embodiments, the binding agent is an analog of one of the aforementioned agents. In particular embodiments, a binding agent is selected from any one of the agents in Tables 2-9. In some embodiments, the binding agent is selected from the group consisting of AZD0328, ABT-126, AQW-051, Cannabidiol, Cilansetron, PH-399733, FACINICLINE/RG3487/MEM-3454, TC-6987, APN-1125, and TC-5619/AT-101. In some embodiments, the binding agent is selected from the group consisting of ABT-126, AZD-0328, APN-1125, RG3487, TC-6987, and TC-5619.

In particular embodiments, the binding agent is an analog of Cilansetron, e.g. as described by one of the compound formulas 2-7 below in either its R or S enantiomer:

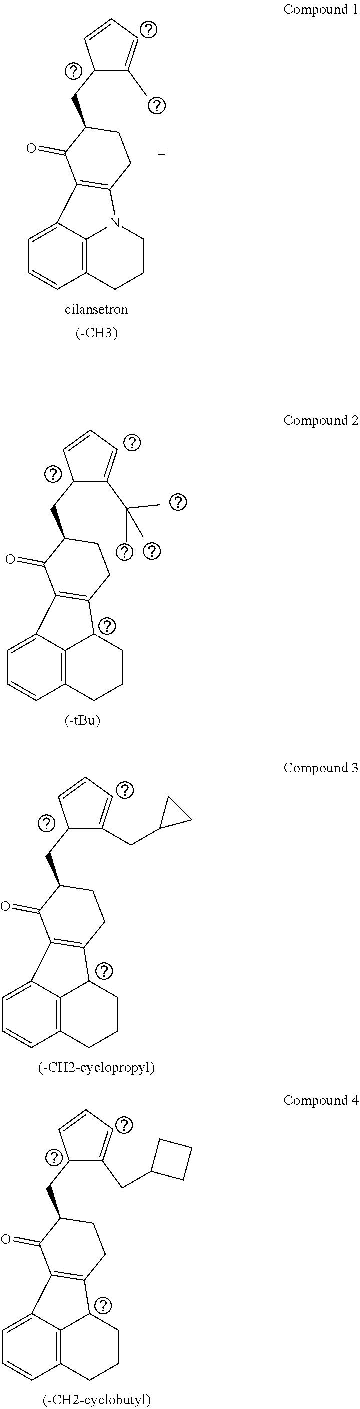 [SMILES: CC1=CC=CC1C[C@H]1CCc2c(c3cccc4c3n2CCC4)C1=O ; CC(C)(C)C1=CC=CC1C[C@H]1CCC2=C(C1=O)c1cccc3c1C2CCC3 ; O=C1C2=C(CC[C@@H]1CC1C=CC=C1CC1CC1)C1CCCc3cccc2c31 ; O=C1C2=C(CC[C@@H]1CC1C=CC=C1CC1CCC1)C1CCCc3cccc2c31]

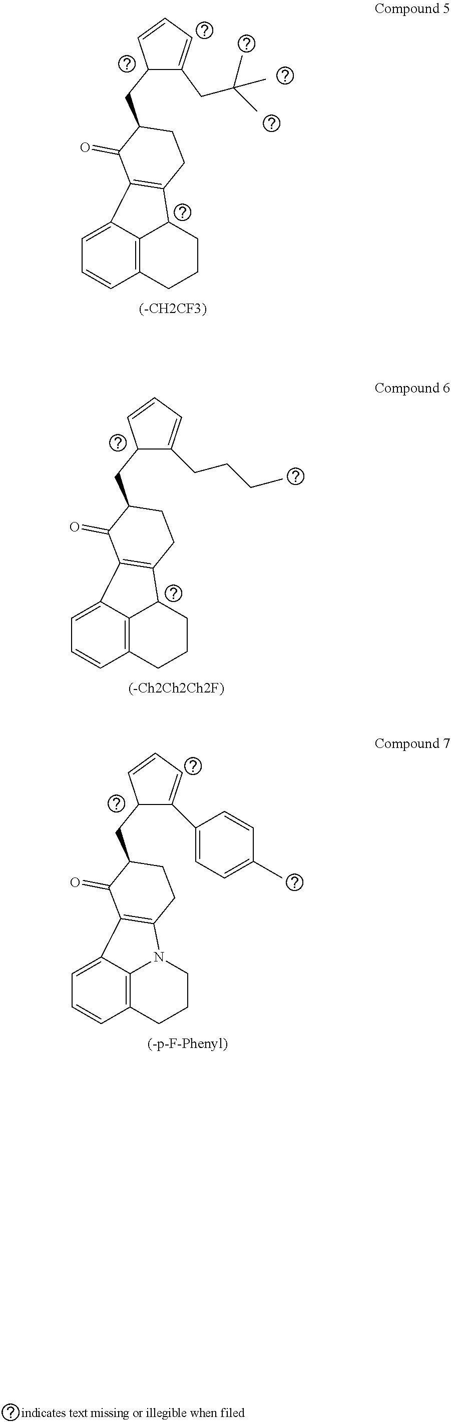 [SMILES: CC(C)(C)CC1=CC=CC1C[C@H]1CCC2=C(C1=O)c1cccc3c1C2CCC3 ; CCCCC1=CC=CC1C[C@H]1CCC2=C(C1=O)c1cccc3c1C2CCC3 ; Cc1ccc(C2=CC=CC2C[C@H]2CCc3c(c4cccc5c4n3CCC5)C2=O)cc1]

In some embodiments, the binding agent acts as an agonist. The term “agonist” as used herein refers to a ligand or binding agent that induces a signaling response. In some embodiments, the binding agent acts as an antagonist. The term antagonist is used herein to refer to an agent that inhibits a signaling response.

In some embodiments, the binding agent is an anxiolytic, anticonvulsant, antidepressant, antipsychotic, antiemetic, nootropic, antibiotic, antifungal, antiviral, or an antiparasitic.

TABLE 2
Binding agents for Glycine Receptor (GlyR)

| Agonists | | Modulators/Binders | Antagonists | |
|---|---|---|---|---|
| Bilobalide | L-Serine | Gavestinel | 468816 | Lindane |
| Cannabidiol | MDL-27531 | Halothane | ACEA-2085 | MDL-100748 |
| D-Alanine | Methoxyflurane | | Bicuculline | MDL-102288 |
| D-Serine | Milacemide | | Brucine | MDL-105519 |
| Desflurane | Moxidectin | | Caffeine | PD-165650 |
| Doramectin | NRX-1050 | | Gavestinel | Picrotoxin |
| Emamectin | NRX-1060 | | Ginkgo biloba | Strychnine |
| Eprinomectin | P-9939 | | GV-196771 | Thiocolchicoside |
| Ethanol | Quisqualamine | | GW 468816 | Tutin |
| Glycine | Rapastinel | | HMR-2371 | UK-315716 |
| Hypotaurine | S-18841 | | L-695902 | ZD-9379 |
| Isoflurane | Sarcosine | | L-701324 | |
| Ivermectin | Sevoflurane | | | |
| L-Alanine | Taurine | | | |
| L-Proline | β-Alanine | | | |

TABLE 3
Binding Agents for λ-Aminobutyric Acid A Receptor (GABA-A)

| Agonists | Modulators/Binders | | | Antagonists |
|---|---|---|---|---|
| 3-acyl-4- | (−)Epigallo- | Etifoxine | Pentobarbital | (±)-cis-(3- |
| quinolone | catechin-3- | | | Aminocyclopentyl) |
| | Gallate | | | butylphosphinic acid |
| Acamprosate | 10-Methoxy- | Etizolam | Petrichloral | (S)-(4-Aminocyclopent-1- |
| | yangonin | | | enyl)butylphosphinic acid |
| Alfadolone | 11-Hydroxy- | Etomidate | PF-4480682 | Amoxapine |
| | yangonin | | | |
| Bamaluzole | 11-Methoxy-12- | Evt-201 | Phenazepam | Bicuculline |
| | Hydroxy- | | | |
| | dehydrokavain | | | |
| Basmisanil | 11-Methoxy- | Fasiplon | Phenobarbital | CGP-36742 (3- |
| | yangonin | | | aminopropyl-n-butyl- |
| | | | | phosphinic acid) |
| Bretazenil | 123i-Iomazenil | Fg-8205 | Pinazepam | Flumazenil |
| CACA | 2-Oxoquazepam | Fletazepam | Pipequaline | Gabazine |
| CAMP | 3-Hydroxy- | Flubromazepam | Pivoxazepam | Ginkgo biloba |
| | phenazepam | | | |
| CP-409092 | 5-Hydroxykavain | Flubromazolam | Potassium | Lindane |
| | | | Bromide | |
| Doramectin | 5,6-Dehydro- | Fludiazepam | Prazepam | Methohexital |
| | methysticin | | | |
| Emamectin | 5,6-Dihydro- | Flumazenil | Premazepam | Picrotoxin |
| | yangonin | | | |
| Eprinomectin | 5,6,7,8- | Flunitrazepam | Primidone | SKF-97541 (3- |
| | Tetrahydro- | | | Aminopropyl |
| | yangonin | | | (methyl)phosphinic acid) |
| Eszopiclone | 7,8- | Flurazepam | Proflazepam | TPMPA |
| | Dihydrokavain | | | |
| Ethanol | 7,8-Dihydro- | Flutazolam | Propanidid | ZAPA ((Z)-3- |
| | methysticin | | | [(Aminoiminomethyl)thio] |
| | | | | prop-2-enoic acid) |
| Etomidate | 7,8-Dihydro- | Flutemazepam | Propofol | |
| | yangonin | | | |
| Flunitrazepam | Abecarnil | Flutoprazepam | PWZ-007A | |
| GABA | Adinazolam | Fosazepam | PWZ-009A1 | |
| Gabamide | Allobarbital | Fospropofol | Pwz-029 | |
| GABOB | Allo- | Ganaxolone | Pyrazolam | |
| | pregnanolone | | | |
| Gaboxadol | Alphaxolone | Gbld-345 | PZ-II-028 | |
| Gamma | Alphenal | Gedocarnil | PZ-II-029 | |
| Hydroxybutyric | | | | |
| Acid | | | | |
| Glutethimide | Alpidem | Gidazepam | Qh-Ii-66 | |
| Ibotenic acid | Alprazolam | Girisopam | Quazepam | |
| Imidazenil | Amentoflavone | Glutethimide | Quinidine | |
| | | | Barbiturate | |
| Isoflurane | Amobarbital | Gyki-52466 | Reclazepam | |
| Isoguvacine | Apigenin | Gyki-52895 | Remimazolam | |
| Isonipecotic | Aprobarbital | Halazepam | Rilmazafone | |
| acid | | | | |
| Ivermectin | Arfendazam | Haloxazolam | Ripazepam | |
| L-830982 | Avizafone | Heptabarbital | Ro15-4513 | |
| Meprobamate | AZD7325 | Hexobarbital | Ro48-6791 | |
| Methoxyflurane | Baicalein | Iclazepam | Ro48-8684 | |
| MK- | Baicalin | Imidazenil | Ro4938581 | |
| 0777/L83098 | | | | |
| Methyprylon | Barbital | Indiplon | Rwj-51204 | |
| Moxidectin | Barbituric Acid | Irazepine | Saripidem | |
| | Derivative | | | |
| Muscimol | Bentazepam | Kavain | Sarmazenil | |
| N4- | Brallobarbital | Kenazepine | Sb-205,384 | |
| Chloroacetyl- | | | | |
| cytosine | | | | |
| arabinoside | | | | |
| Pagoclone | Bretazenil | Ketazolam | Scutellarein | |
| Phenibut | Bromazepam | L-655708 | Secobarbital | |
| Picamilon | Brotizolam | L-838,417 | Sevoflurane | |
| Piperazine | Butalbital | Lanthanum | Sh-053-R-Ch3- | |
| | | | 2′F | |
| Piperidine-4- | Butethal | LAU 156 | Skull- | |
| sulfonic acid | | | capflavone II | |
| Progabide | Butobarbital | LAU 157 | Sl-651,498 | |
| QH-ii-066 | Camazepam | LAU 159 | Sodium Amytal | |
| Quisqualamine | Carburazepam | LAU 161 | Sodium | |
| | | | Pentothal | |
| Sevoflurane | Carisoprodol | LAU 162 | Stiripentol | |
| SL 75102 | CGS 20625 | LAU 163 | Sulazepam | |
| SL-651,498 | CGS 20625 | LAU 176 | Sulfonmethane | |
| Thiamylal | CGS 8216 | LAU 177 | Suproclone | |
| Thiomuscimol | CGS 9895 | LAU 206 | Suriclone | |
| Tolgabide | CGS 9896 | Lofendazam | Sx-3228 | |
| Topiramate | Chloral Hydrate | Lopirazepam | Talampanel | |
| Zolpidem | Chloralose | Loprazolam | Talbutal | |
| α5IA | Chlordiazepoxide | Lorazepam | Taniplon | |
| | Chlormezanone | Lorbamate | Temazepam | |
| | Chloroform | Loreclezole | Tetrazepam | |
| | Ciclotizolam | Lorediplon | Tetronal | |
| | Cinazepam | Lormetazepam | Thdoc | |
| | Cinolazepam | Meclonazepam | Theanine | |
| | Cl-218,872 | Medazepam | Thiamylal | |
| | Clazolam | Menitrazepam | Thieno- | |
| | | | diazepine | |
| | Climazolam | Mephobarbital | Thiopental | |
| | Clobazam | Meprobamate | Tofisopam | |
| | Clomethiazole | Metaclazepam | Tolufazepam | |
| | Clonazepam | Methaqualone | Tp-003 | |
| | Clonazolam | Metharbital | Tp-13 | |
| | Clorazepate | Methohexital | Tpa-023 | |
| | Clotiazepam | Methyl- | Triazolam | |
| | | phenobarbital | | |
| | Cloxazolam | Methyprylon | Triflubazam | |
| | Cp-1414s | Methysticin | Triflunor- | |
| | | | dazepam | |
| | CTP-354 | Metizolam | Trional | |
| | Cyclobarbital | Mexazolam | Tuclazepam | |
| | Cyprazepam | Midazolam | Uldazepam | |
| | Delorazepam | Motrazepam | Valerenic Acid | |
| | Demoxepam | N-Desalkyl- | Valeric Acid | |
| | | flurazepam | | |
| | Deschloro- | Necopidem | Wogonin | |
| | etizolam | | | |
| | Desmethoxy- | Nerisopam | XHe-II-006 | |
| | yangonin | | | |
| | Desmethyl- | Niacin | XHe-II-019 | |
| | flunitrazepam | | | |
| | Diazepam | Niacinamide | XHe-II-087c | |
| | Diclazepam | Nifoxipam | XHe-II-094 | |
| | Diethyl Ether | Nimetazepam | XHe-II-098b | |
| | Dihydro- | Nitrazepam | XHe-II-17 | |
| | ergotoxine | | | |
| | Dihydroquinidine | Nitrazepate | XHe-III-006c | |
| | Barbiturate | | | |
| | Diproqualone | Nitrazolam | XHe-III-063 | |
| | Divaplon | Nordazepam | XHe-III-24 | |
| | Doxefazepam | Nortetrazepam | XHe/ON-I | |
| | Elb-139 | Ns-2664 | Y-23684 | |
| | Elfazepam | Ns-2710 | Yangonin | |
| | Estazolam | Ocinaplon | Zaleplon | |
| | Eszopiclone | Oroxylin A | Zapizolam | |
| | Etaqualone | Oxazepam | Zinc | |
| | Etazepine | Oxazolam | Zk-93423 | |
| | Etazolate | Pagoclone | Zolazepam | |
| | Ethyl | Panadiplon | Zolpidem | |
| | Carfluzepate | | | |
| | Ethyl Dirazepate | Pazinaclone | Zomebazam | |
| | Ethyl Loflazepate | PB-XHe | Zopiclone | |

TABLE 4
Binding Agents for 5-Hydroxytryptamine Receptor (5-HT3)

| | Modulators/ | | |
|---|---|---|---|
| Agonists | Binders | Antagonists | |
| 2-methyl-5-HT | 5-chloroindole | 3-Tropanyl indole-3- | Mianserin |
| | | carboxylate | |
| Alpha- | Trimipramine | Adr-851 | Mirisetron |
| Methyltryptamine | | | Maleate |
| Bufotenin | | Adr-882 | Mirtazapine |
| Chlorophenyl- | | Alosetron | Ml-1035 |
| biguanide | | | |
| Cisapride | | Amoxapine | Mm-218 |
| DDP733 | | Anpirtoline | N-3256 |
| Ethanol | | Aripiprazole | Netupitant |
| Ethanol | | AS-8112 | Olanzapine |
| Ibogaine | | Azasetron | Ondansetron |
| Metoclopramide | | Batanopride | Org-4419 |
| Phenylbiguanide | | Bimu-1 | Org-4419-2 |
| Quipazine | | Chloroprocaine | Palonosetron |
| RS-56812 | | Cilansetron | Pancopride |
| SR-57227 | | Clozapine | Procaine |
| Tapentadol | | Cp-81386 | Quetiapine |
| Varenicline | | Cp-93318 | R-093777 |
| YM-31636 | | Cr-3124 | R-Zacopride |
| | | DA-9701 | Ramosetron |
| | | Daizac | Renzapride |
| | | Dat-582 | Rg-12915 |
| | | Dau-6285 | Ricasetron |
| | | Ddp-225 | Rocuronium |
| | | Dolasetron | Rs-16566 |
| | | E-3620 | Rs-33800 |
| | | Fabesetron | Rs-56532 |
| | | Facinicline/RG3487 | Rs-56812 |
| | | Hydrochloride | |
| | | Galdansetron | S-21007 |
| | | Gastroprokinetics | Sc-50410 |
| | | Gk-128 | Sc-52150 |
| | | Gr-65630 | Sc-52246 |
| | | Granisetron | Sc-52491 |
| | | Gyki-46903 | Sc-54750 |
| | | Itasetron | Sdz-Icm-567 |
| | | Kb-6806 | Sep-226332 |
| | | Kf-18259 | Srss-021 |
| | | Kf-20170 | Tedatioxetine |
| | | Kga-0941 | Thujone |
| | | L-683877 | Topiramate |
| | | Lamotrigine | Tropisetron |
| | | Lerisetron | Tubocurarine |
| | | Lintopride | Tzb-30878 |
| | | Litoxetine | Va-21B7 |
| | | Loxapine | Vi-0134 |
| | | Lurosetron | Vortioxetine |
| | | Ly-278584 | Vortioxetine |
| | | | Hydrobromide |
| | | M1, the major active | Way-100289 |
| | | metabolite of mosapride | |
| | | Mci-225 | Ym-114 |
| | | Mci-225 | Ym-26103-2 |
| | | Memantine | Ym-26308-2 |
| | | Menthol | Zacopride |
| | | Methadone | Zatosetron |
| | | Metoclopramide | Ziprasidone |

TABLE 5
Binding Agents for Nicotinic Acetylcholine Receptor (nAchR)

| Agonists | Modulators/Binders | Antagonists |
|---|---|---|
| (+)-N-(1-azabicyclo[2.2.2]oct-3- | A-867744 | (−)-7-methyl-2-exo-[3′-(6- |
| yl)benzo[b]furan-2-carboxamide | | [18F]fluoropyridin-2-yl)-5′- |
| | | pyridinyl]-7-azabicyclo[2.2.1]heptane |
| 3-Bromocytisine | AQW-051 | 2-fluoro-3-(4-nitro- |
| | | phenyl)deschloroepibatidine |
| A-366,833 | AVL-3288 | Acv-1 |
| A-582941 | Desformylflustrabromine | ACVx |
| A-82695 | Ethanol | Amobarbital |
| A-84,543 | Galantamine | Anandamide |
| ABT-089 | Ivermectin | Anq-9040 |
| ABT-126 | Nefiracetam | Aprobarbital |
| Abt-202 | NS-1738 | ATG-001 |
| ABT-418 | NS-9283 | ATG003 |
| Abt-418 | PNU-120,596 | Atracurium besylate |
| Abt-560 | Tetraethylammonium | Barbital |
| ABT-894 | | Barbituric acid derivative |
| Acetylcholine | | Biperiden |
| Altinicline | | Bupropion |
| Altinicline | | Butabarbital |
| Aminoethoxypyridine | | Butalbital |
| Anabasine | | Butethal |
| AR-R17779 | | Chloroprocaine |
| Asm-024 | | Cisatracurium Besilate |
| AZD-0328 | | Cisatracurium besylate |
| Bupropion Hydrochloride | | Coclaurine |
| Carbachol | | Dehydronorketamine |
| Choline | | Deuterated Bupropion |
| Cm-2433 | | Dextromethorphan |
| CP-601927 | | Doxacurium chloride |
| CP-601932 | | Ethanol |
| Cp-810123 | | Gallamine Triethiodide |
| Cytisine | | Heptabarbital |
| DBO-83 | | Hexobarbital |
| Decamethonium | | Hydroxybupropion |
| Dianicline | | Hydroxynorketamine |
| Dianicline | | Inaperisone |
| Encenicline | | Iptakalim |
| Epibatidine | | Isoflurane |
| EVP-6124 | | Ketamine |
| Evp-6124 | | Kynurenic acid |
| Galantamine | | Levomethadyl Acetate |
| GTS-21 | | Lobeline |
| Gts-21 | | Mecamylamine |
| ICH-3 | | Mecamylamine |
| Ispronicline | | Memantine |
| Levamisole | | Methadone |
| Lobeline | | Metharbital |
| Lobeline Sulphate | | Methyllycaconitine |
| Mem-3454 | | Methylphenobarbital |
| MEM-63908 | | Metocurine |
| Mem-63908 | | Metocurine Iodide |
| N-(3-pyridinyl)-bridged bicyclic | | Mivacurium |
| diamine | | |
| Nicotine | | Neramexane |
| PH-399733 | | Nic-002 |
| Ph-399733 | | Nitrous oxide |
| PHA-543,613 | | Norketamine |
| Pha-543613 | | Org-9991 |
| PHA-709829 | | Pancuronium |
| PNU-282,987 | | Pentobarbital |
| Pnu-282987 | | Pentolinium |
| Pozanicline | | Phenobarbital |
| Pozanicline | | Pipecuronium |
| Rivanicline | | Pipecuronium |
| Rivanicline | | PNU-120,596 |
| Rjr-1401 | | Primidone |
| Sar-130479 | | Procaine |
| Sazetidine A | | Quinolizidine (−)-1-epi-207I |
| Sib-1663 | | RJR-2531 |
| Sib-1765f | | Rocuronium |
| Sib-3182 | | RPI-78M |
| Sofinicline | | Secobarbital |
| SSR-180,711 | | Talbutal |
| Succinylcholine | | Thiopental |
| Suvn-F90101 | | Trimethaphan |
| Suvn-F91201 | | Tubocurarine |
| Tacrine-huperzine A | | U-2902 |
| TC-1698 | | Vecuronium |
| TC-1827 | | α-Bungarotoxin |
| Tc-2216 | | α-Conotoxin |
| Tc-2403 | | |
| Tc-2429 | | |
| Tc-2559 | | |
| Tc-2696 | | |
| TC-5619 | | |
| Tebanicline | | |
| Tropisetron | | |
| Ub-165 | | |
| Varenicline | | |
| WAY-317,538 | | |
| APN-1125 | | |

TABLE 6
Binding Agents for ATP-Gated P2X Receptor Cation Channel (P2X)

| | Modulators/ | | |
|---|---|---|---|
| Agonists | Binders | Antagonists | |
| ATP | Ivermectin | 4-benzoyl-1-substituted- | MK-801 |
| | | piperazin-2-ones | |
| BzATP | | 5-methyl-6, 7-dihydro- | MRS2159 |
| | | 5Hcyclopentapyrazine | |
| α,β-meATP | | 5-oxo-3- | NF023 |
| | | pyrrolidinecarboxamides | |
| | | 5,6,7,8-tetrahydropyrido [4,3,d]- | NF279 |
| | | pyrimidines | |
| | | A-317491 | NF449 |
| | | Amitripty line | Nortripty line |
| | | Azaindole-3-carboxamides | Oxoisoquinoline carboxamides |
| | | AZD-9056 | Paroxetine |
| | | Benzamide 6 | Piperazines |
| | | Benzofuro-1,4-diazepin-2-ones | Polycyclic guanines |
| | | Benzoimidazoles | PPADS |
| | | Biaryl benzamides | PPNDS |
| | | Carboxamides | Pyrazolo-[1,5,a]-pyridine |
| | | | carboxamides |
| | | CE-224535 | Pyridazinones |
| | | Desipramine | Pyridoxal-5-pho sphate |
| | | Diaminopyridines | Pyrrolinones |
| | | Doxepin | Pyrrolo-[2,3,b]-pyridine |
| | | | carboxamides |
| | | Eslicarbazepine acetate | Pyrrolopy rimidin-7-ones |
| | | EVT-401 | Quinoline carboxamides |
| | | Fluoxetine | RO-3 |
| | | Hyaluronic acid derivatives | RO-4 |
| | | Imipramine | RO-51 |
| | | Indole carboxamides | Spinorphins |
| | | Indole-3-carboxamides | Suramin |
| | | Ip5I | Tetrahydro-2H-1,2-thiazine |
| | | | 1,1-dioxides |
| | | Isoquinoline carboxamides | TNP-ATP |
| | | Isothiazolidine 1,1-dioxides | |

TABLE 7
Binding Agents for Inwardly Rectifying Potassium Channel (Kir)

| Agonists | Modulators/Binders | Antagonists |
|---|---|---|
| Diazoxide | | Acetohexamide |
| Iptakalim | | Carbutamide |
| Minoxidil | | Chlorpropamide |
| Nicorandil | | Glibenclamide |
| Phosphatidylinositol | | Glibenclamide |
| 4,5-Bisphosphate | | |
| Pinacidil | | Glibornuride |
| | | Gliclazide |
| | | Glimepiride |
| | | Glipizide |
| | | Gliquidone |
| | | Glisoxepide |
| | | Glyburide |
| | | Glyclopyramide |
| | | Glycyclamide |
| | | Metahexamide |
| | | Tolazamide |
| | | Tolbutamide |
| | | Tolhexamide |

TABLE 8
Binding Agents for Voltage Dependent Potassium Channel (KCNQ/Kv7)

| Agonists | Modulators/Binders | Antagonists |
|---|---|---|
| Diazoxide | | Azimilide |
| Flupirtine | | Amiodarone |
| Minoxidil | | Bretylium |
| Nicorandil | | Clofilium |
| Pinacidil | | Dalfampridine |
| Retigabine | | Dofetilide |
| | | E-4031 |
| | | Ibutilide |
| | | Nifekalant |
| | | Sematilide |
| | | Sotalol |
| | | Sulfonylureas |
| | | Tedisamil |

TABLE 9
Binding Agents for Cystic Fibrosis Transmembrane
Conductance Regulator (CFTR)

| Agonists | Modulators/Binders | Antagonists |
|---|---|---|
| | 8-cyclopentyl-1,3-dipropylxanthine | Bumetanide |
| | 8-methoxypsoralen | Crofelemer |
| | Apigenin | Glyburide |
| | CTP-656 | Ibuprofen |
| | Genistein | |
| | IB MX | |
| | Ivacaftor | |
| | Lumacaftor | |

### E. Polynucleotides

In various illustrative embodiments, the present disclosure contemplates, in part, polynucleotides, polynucleotides encoding engineered receptor polypeptides including LGICs, and subunits and muteins thereof, and fusion polypeptides, viral vector polynucleotides, and compositions comprising the same.

As used herein, the terms “polynucleotide,” “nucleotide,” “nucleotide sequence” or “nucleic acid” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. Polynucleotides may be deoxyribonucleic acid (DNA), ribonucleic acid (RNA) or DNA/RNA hybrids. Polynucleotides may be single-stranded or double-stranded. Polynucleotides include, but are not limited to: pre-messenger RNA (pre-mRNA), messenger RNA (mRNA), RNA, short interfering RNA (siRNA), short hairpin RNA (shRNA), microRNA (miRNA), ribozymes, synthetic RNA, genomic RNA (gRNA), plus strand RNA (RNA(+)), minus strand RNA (RNA(−)), synthetic RNA, genomic DNA (gDNA), PCR amplified DNA, complementary DNA (cDNA), synthetic DNA, or recombinant DNA. Polynucleotides refer to a polymeric form of nucleotides of at least 5, at least 10, at least 15, at least 20, at least 25, at least 30, at least 40, at least 50, at least 100, at least 200, at least 300, at least 400, at least 500, at least 1000, at least 5000, at least 10000, or at least 15000 or more nucleotides in length, either ribonucleotides or deoxynucleotides or a modified form of either type of nucleotide, as well as all intermediate lengths. It will be readily understood that “intermediate lengths” in this context, means any length between the quoted values, such as 6, 7, 8, 9, etc., 101, 102, 103, etc.; 151, 152, 153, etc.; 201, 202, 203, etc. In particular embodiments, polynucleotides or variants have at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference sequence described herein or known in the art, typically where the variant maintains at least one biological activity of the reference sequence unless otherwise stated.

As used herein, the term “gene” may refer to a polynucleotide sequence comprising enhancers, promoters, introns, exons, and the like. In particular embodiments, the term “gene” refers to a polynucleotide sequence encoding a polypeptide, regardless of whether the polynucleotide sequence is identical to the genomic sequence encoding the polypeptide.

As used herein, a “cis-acting sequence, “cis-acting regulatory sequence”, or “cis-acting nucleotide sequence” or equivalents refers to a polynucleotide sequence that is associated with the expression, e.g. transcription and/or translation, of a gene. In one embodiment, the cis-acting sequence regulates transcription because it is a binding site for a polypeptide that represses or decreases transcription or a polynucleotide sequence associated with a transcription factor binding site that contributes to transcriptional repression. Examples of cis-acting sequences that regulate the expression of polynucleotide sequences and that may be operably linked to the polynucleotides of the present disclosure to regulate the expression of the subject engineered receptors are well known in the art and include such elements as promoter sequences (e.g. CAG, CMV, SYN, CamKII, TRPV1), Kozak sequences, enhancers, posttranscriptional regulatory elements, miRNA binding elements, and polyadenylation sequences.

As one non-limiting example, a promoter sequence is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes. Various promoters may be used to drive the various vectors of the present invention. For example, the promoter may be a constitutively active promoter, i.e. a promoter that is active in the absence externally applied agents, e.g. the CMV IE1 promoter, the SV40 promoter, GAPDH promoter, Actin promoter. The promoter may be an inducible promoter, i.e. a promoter whose activity is regulated upon the application of an agent to the cell, e.g. doxycycline, the tet-on or tet-off promoter, the estrogen receptor promoter, etc. The promoter may be a tissue-specific promoter, i.e. a promoter that is active on certain types of cells.

In some embodiments, the promoter is active in an excitable cell. By an “excitable cell”, it is meant a cell that is activated by a change in membrane potential, e.g. a neuron or myocyte, e.g. a dorsal root ganglion neuron, a motor neuron, an excitatory neuron, an inhibitory neuron, or a sensory neuron. Promoters that are active in an excitable cell that would find use in the present polynucleotide compositions would include neuronal promoters, for example, the synapsin (SYN), TRPV1, Naᵥ1 0.7, Naᵥ1 0.8, Naᵥ1 0.9, CamKII, NSE, and Advillin promoters; myocyte promoters, e.g. the desmin (Des), alpha-myosin heavy chain (α-MHC), myosin light chain 2 (MLC-2) and cardiac troponin C (cTnC) promoters; and ubiquitous acting promoters, e.g. CAG, CBA, E1Fa, Ubc, CMV, and SV40 promoters.

As used herein, a “regulatory element for inducible expression” refers to a polynucleotide sequence that is a promoter, enhancer, or functional fragment thereof that is operably linked to a polynucleotide to be expressed and that responds to the presence or absence of a molecule that binds the element to increase (turn-on) or decrease (turn-off) the expression of the polynucleotide operably linked thereto. Illustrative regulatory elements for inducible expression include, but are not limited to, a tetracycline responsive promoter, an ecdysone responsive promoter, a cumate responsive promoter, a glucocorticoid responsive promoter, an estrogen responsive promoter, an RU-486 responsive promoter, a PPAR-γ promoter, and a peroxide inducible promoter.

A “regulatory element for transient expression” refers to a polynucleotide sequence that can be used to briefly or temporarily express a polynucleotide nucleotide sequence. In particular embodiments, one or more regulatory elements for transient expression can be used to limit the duration of a polynucleotide. In certain embodiments, the preferred duration of polynucleotide expression is on the order of minutes, hours, or days. Illustrative regulatory elements for transient expression include, but are not limited to, nuclease target sites, recombinase recognition sites, and inhibitory RNA target sites. In addition, to some extent, in particular embodiments, a regulatory element for inducible expression may also contribute to controlling the duration of polynucleotide expression.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion, substitution, or modification of at least one nucleotide. Accordingly, the terms “polynucleotide variant” and “variant” include polynucleotides in which one or more nucleotides have been added or deleted, or modified, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide. In particular embodiments, polynucleotides or variants have at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference sequence described herein or known in the art, typically where the variant maintains at least one biological activity of the reference sequence unless otherwise stated.

In one embodiment, a polynucleotide comprises a nucleotide sequence that hybridizes to a target nucleic acid sequence under stringent conditions. To hybridize under “stringent conditions” describes hybridization protocols in which nucleotide sequences at least 60% identical to each other remain hybridized. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Terms used to describe sequence relationships between two or more polynucleotides or polypeptides include “reference sequence,” “comparison window,” “sequence identity,” “percentage of sequence identity,” and “substantial identity.” A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., Current Protocols in Molecular Biology, John Wiley & Sons Inc, 1994-1998, Chapter 15.

An “isolated polynucleotide,” as used herein, refers to a polynucleotide that has been purified from the sequences which flank it in a naturally-occurring state, e.g., a DNA fragment that has been removed from the sequences that are normally adjacent to the fragment. In particular embodiments, an “isolated polynucleotide” refers to a complementary DNA (cDNA), a recombinant DNA, or other polynucleotide that does not exist in nature and that has been made by the hand of man.

Terms that describe the orientation of polynucleotides include: 5′ (normally the end of the polynucleotide having a free phosphate group) and 3′ (normally the end of the polynucleotide having a free hydroxyl (OH) group). Polynucleotide sequences can be annotated in the 5′ to 3′ orientation or the 3′ to 5′ orientation. For DNA and mRNA, the 5′ to 3′ strand is designated the “sense,” “plus,” or “coding” strand because its sequence is identical to the sequence of the pre-messenger (premRNA) [except for uracil (U) in RNA, instead of thymine (T) in DNA]. For DNA and mRNA, the complementary 3′ to 5′ strand which is the strand transcribed by the RNA polymerase is designated as “template,” “antisense,” “minus,” or “non-coding” strand. As used herein, the term “reverse orientation” refers to a 5′ to 3′ sequence written in the 3′ to 5′ orientation or a 3′ to 5′ sequence written in the 5′ to 3′ orientation.

The term “flanked” refers to a polynucleotide sequence that is in between an upstream polynucleotide sequence and/or a downstream polynucleotide sequence, i.e., 5′ and/or 3′, relative to the sequence. For example, a sequence that is “flanked” by two other elements (e.g., ITRs), indicates that one element is located 5′ to the sequence and the other is located 3′ to the sequence; however, there may be intervening sequences therebetween.

The terms “complementary” and “complementarity” refer to polynucleotides (i.e., a sequence of nucleotides) related by the base-pairing rules. For example, the complementary strand of the DNA sequence 5′ AGTC AT G 3′ is 3′ TCAGTAC 5′. The latter sequence is often written as the reverse complement with the 5′ end on the left and the 3′ end on the right, 5′ C A T G A C T 3′. A sequence that is equal to its reverse complement is said to be a palindromic sequence. Complementarity can be “partial,” in which only some of the nucleic acids' bases are matched according to the base pairing rules. Or, there can be “complete” or “total” complementarity between the nucleic acids.

The terms “nucleic acid cassette” or “expression cassette” as used herein refers to polynucleotide sequences within a larger polynucleotide, such as a vector, which are sufficient to express one or more RNAs from a polynucleotide. The expressed RNAs may be translated into proteins, may function as guide RNAs or inhibitory RNAs to target other polynucleotide sequences for cleavage and/or degradation. In one embodiment, the nucleic acid cassette contains one or more polynucleotide(s)-of-interest. In another embodiment, the nucleic acid cassette contains one or more expression control sequences operably linked to one or more polynucleotide(s)-of-interest. Polynucleotides include polynucleotide(s)-of-interest. As used herein, the term “polynucleotide-of-interest” refers to a polynucleotide encoding a polypeptide or fusion polypeptide or a polynucleotide that serves as a template for the transcription of an inhibitory polynucleotide, e.g., LGICs, and subunits and muteins thereof, as contemplated herein. In a particular embodiment, a polynucleotide-of-interest encodes a polypeptide or fusion polypeptide having one or more enzymatic activities, such as a nuclease activity and/or chromatin remodeling or epigenetic modification activities.

Vectors may comprise 1, 2, 3, 4, 5, 6, 7, 8, 9 or 10 or more nucleic acid cassettes. In a preferred embodiment of the disclosure, a nucleic acid cassette comprises one or more expression control sequences (e.g., a promoter or enhancer operable in a neuronal cell) operably linked to a polynucleotide encoding a engineered receptor, e.g., an LGIC, or subunit or muteins thereof. The cassette can be removed from or inserted into other polynucleotide sequences, e.g., a plasmid or viral vector, as a single unit.

In one embodiment, a polynucleotide contemplated herein comprises 1, 2, 3, 4, 5, 6, 7, 8, 9, or more nucleic acid cassettes any number or combination of which may be in the same or opposite orientations.

Moreover, it will be appreciated by those of ordinary skill in the art that, as a result of the degeneracy of the genetic code, there are many nucleotide sequences that may encode a polypeptide, or fragment of variant thereof, as contemplated herein. Some of these polynucleotides bear minimal homology to the nucleotide sequence of any native gene. Nonetheless, polynucleotides that vary due to differences in codon usage are specifically contemplated by the present disclosure, for example polynucleotides that are optimized for human and/or primate codon selection. In one embodiment, polynucleotides comprising particular allelic sequences are provided. Alleles are endogenous polynucleotide sequences that are altered as a result of one or more mutations, such as deletions, additions and/or substitutions of nucleotides.

### F. Vectors

In some aspects of the disclosure, a nucleic acid molecule, i.e., a polynucleotide encoding an engineered receptor is delivered to a subject. In some cases, the nucleic acid molecule encoding the engineered receptor is delivered to a subject by a vector. In various embodiments, a vector comprises a one or more polynucleotide sequences contemplated herein. The term “vector” is used herein to refer to a nucleic acid molecule capable of transferring or transporting another nucleic acid molecule. The transferred polynucleotide is generally linked to, e.g., inserted into, the vector nucleic acid molecule. A vector may include sequences that direct autonomous replication in a cell, or may include sequences sufficient to allow integration into host cell DNA. A vector can deliver a target polynucleotide to an organism, a cell or a cellular component. In some cases, the vector is an expression vector. An “expression vector” as used herein refers to a vector, for example, a plasmid, that is capable of promoting expression, as well as replication of a polynucleotide incorporated therein. Typically, the nucleic acid sequence to be expressed is operably linked to cis-acting regulatory sequence, e.g. a promoter and/or enhancer sequence, and is subject to transcription regulatory control by the promoter and/or enhancer. In particular cases, a vector is used to deliver a nucleic acid molecule encoding an engineered receptor of the disclosure to a subject.

In particular embodiments, any vector suitable for introducing an expression cassette or polynucleotide encoding an engineered receptor into a neuronal cell can be employed. Illustrative examples of suitable vectors include plasmids (e.g., DNA plasmids or RNA plasmids), transposons, cosmids, bacterial artificial chromosomes, and viral vectors. In some cases, the vector is a circular nucleic acid, for e.g., a plasmid, a BAC, a PAC, a YAC, a cosmid, a fosmid, and the like. In some cases, circular nucleic acid molecules can be utilized to deliver a nucleic acid molecule encoding an engineered receptor to a subject. For example, a plasmid DNA molecule encoding an engineered receptor can be introduced into a cell of a subject whereby the DNA sequence encoding the engineered receptor is transcribed into mRNA and the mRNA “message” is translated into a protein product. The circular nucleic acid vector will generally include regulatory elements that regulate the expression of the target protein. For example, the circular nucleic acid vector may include any number of promoters, enhancers, terminators, splice signals, origins of replication, initiation signals, and the like.

In some cases, the vector can include a replicon. A replicon may be any nucleic acid molecule capable of self-replication. In some cases, the replicon is an RNA replicon derived from a virus. A variety of suitable viruses (e.g. RNA viruses) are available, including, but not limited to, alphavirus, picornavirus, flavivirus, coronavirus, pestivirus, rubivirus, calcivirus, and hepacivirus.

In some embodiments, the vector is a non-viral vector. By a “non-viral vector”, it is meant any delivery vehicle that does not comprise a viral capsid or envelope, e.g. lipid nanoparticles (anionic (negatively charged), neutral, or cationic (positively charged)), heavy metal nanoparticles, polymer-based particles, plasmid DNA, minicircle DNA, minivector DNA, ccDNA, synthetic RNA, exosomes, and the like. Non-viral vectors may be delivered by any suitable method as would be well understood in the art, including, e.g., nanoparticle delivery, particle bombardment, electroporation, sonication, or microinjection. See, e.g. Chen et al. Mol. Therapy, Methods and Clinical Development. 2016 January; Vol 3, issue 1; and Hardy, C E et al. Genes (Basel). 2017 February; 8(2): 65

In other embodiments, the vector is a viral vector. By a “viral vector” it is meant a delivery vehicle that comprises a viral capsid or envelop surrounding a polynucleotide encoding an RNA or polypeptide of interest. In some cases, the viral vector is derived from a replication-deficient virus. Non-limiting examples of viral vectors suitable for delivering a nucleic acid molecule of the disclosure to a subject include those derived from adenovirus, retrovirus (e.g., lentivirus), adeno-associated virus (AAV), and herpes simplex-1 (HSV-1). Illustrative examples of suitable viral vectors include, but are not limited to, retroviral vectors (e.g., lentiviral vectors), herpes virus based vectors and parvovirus based vectors (e.g., adeno-associated virus (AAV) based vectors, AAV-adenoviral chimeric vectors, and adenovirus-based vectors).

The term “parvovirus” as used herein encompasses all parvoviruses, including autonomously-replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, mouse minute virus, bovine parvovirus, canine parvovirus, chicken parvovirus, feline panleukopenia virus, feline parvovirus, goose parvovirus, and B19 virus. Other autonomous parvoviruses are known to those skilled in the art. See, e.g., Fields et al., 1996 Virology, volume 2, chapter 69 (3d ed., Lippincott-Raven Publishers).

The genus Dependovirus contains the adeno-associated viruses (AAV), including but not limited to, AAV type 1, AAV type 2, AAV type 3, AAV type 4, AAV type 5, AAV type 6, AAV type 7, AAV type 8, AAV type 9, AAV type rh10, avian AAV, bovine AAV, canine AAV, equine AAV, and ovine AAV.

In a preferred embodiment, the vector is an AAV vector. In particular cases, the viral vector is an AAV-6 or AAV-9 vector.

The genomic organization of all known AAV serotypes is similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural (VP) proteins. The VP proteins (VP1, -2 and -3) form the capsid and contribute to the tropism of the virus. The terminal 145 nt ITRs are self-complementary and are organized so that an energetically stable intramolecular duplex forming a T-shaped hairpin may be formed. These hairpin structures function as an origin for viral DNA replication, serving as primers for the cellular DNA polymerase complex. Following wild-type (wt) AAV infection in mammalian cells the Rep genes are expressed and function in the replication of the viral genome.

In some cases, the outer protein “capsid” of the viral vector occurs in nature, e.g. AAV1, AAV2, AAV3, AAV4, AAV5, AAV6, AAV7, AAV8, AAV9, or AAV10. In particular cases, the capsid is synthetically engineered (e.g. through directed evolution or rational design) to possess certain unique characteristics not present in nature such as altered tropism, increased transduction efficiency, or immune evasion. An example of a rationally designed capsid is the mutation of one or more surface-exposed tyrosine (Y), serine (S), threonine (T), and lysine (K) residues on the VP3 viral capsid protein. Non-limiting examples of viral vectors whose VP3 capsid proteins have been synthetically engineered and are amenable for use with the compositions and methods provided herein include: AAV1(Y705+731F+T492V), AAV2(Y444+500+730F+T491V), AAV3(Y705+731F), AAV5(Y436+693+719F), AAV6(Y705+731F+T492V), AAV8(Y733F), AAV9(Y731F), and AAV10(Y733F). Non-limiting examples of viral vectors that have been engineered through directed evolution and are amenable for use with the compositions and methods provided herein include AAV-7m8 and AAV-ShH10.

A “recombinant parvoviral or AAV vector” (or “rAAV vector”) herein refers to a vector comprising one or more polynucleotides contemplated herein that are flanked by one or more AAV ITRs. Such polynucleotides are said to be “heterologous” to the ITRs, as such combinations do not ordinarily occur in nature. Such rAAV vectors can be replicated and packaged into infectious viral particles when present in an insect host cell that is expressing AAV rep and cap gene products (i.e., AAV Rep and Cap proteins). When an rAAV vector is incorporated into a larger nucleic acid construct (e.g., in a chromosome or in another vector such as a plasmid or baculovirus used for cloning or transfection), then the rAAV vector is typically referred to as a “pro-vector” which can be “rescued” by replication and encapsidation in the presence of AAV packaging functions and necessary helper functions.

In particular embodiments, any AAV ITR may be used in the AAV vectors, including ITRs from AAV1, AAV2, AAV3, AAV4, AAV5, AAV6, AAV7, AAV8, AAV9, AAV10, AAV11, AAV12, AAV13, AAV14, AAV15, and AAV16. In one preferred embodiment, an AAV vector contemplated herein comprises one or more AAV2 ITRs.

rAAV vectors comprising two ITRs have a payload capacity of about 4.4 kB. Self-complementary rAAV vectors contain a third ITR and package two strands of the recombinant portion of the vector leaving only about 2.1 kB for the polynucleotides contemplated herein. In one embodiment, the AAV vector is an scAAV vector.

Extended packaging capacities that are roughly double the packaging capacity of an rAAV (about 9 kB) have been achieved using dual rAAV vector strategies. Dual vector strategies useful in producing rAAV contemplated herein include, but are not limited to splicing (trans-splicing), homologous recombination (overlapping), or a combination of the two (hybrid). In the dual AAV trans-splicing strategy, a splice donor (SD) signal is placed at the 3′ end of the 5′-half vector and a splice acceptor (SA) signal is placed at the 5′ end of the 3′-half vector. Upon co-infection of the same cell by the dual AAV vectors and inverted terminal repeat (ITR)-mediated head-to-tail concatemerization of the two halves, trans-splicing results in the production of a mature mRNA and full-size protein (Yan et al., 2000). Trans-splicing has been successfully used to express large genes in muscle and retina (Reich et al., 2003; Lai et al., 2005). Alternatively, the two halves of a large transgene expression cassette contained in dual AAV vectors may contain homologous overlapping sequences (at the 3′ end of the 5′-half vector and at the 5′ end of the 3′-half vector, dual AAV overlapping), which will mediate reconstitution of a single large genome by homologous recombination (Duan et al., 2001). This strategy depends on the recombinogenic properties of the transgene overlapping sequences (Ghosh et al., 2006). A third dual AAV strategy (hybrid) is based on adding a highly recombinogenic region from an exogenous gene (i.e., alkaline phosphatase; Ghosh et al., 2008, Ghosh et al., 2011)) to the trans-splicing vectors. The added region is placed downstream of the SD signal in the 5′-half vector and upstream of the SA signal in the 3′-half vector in order to increase recombination between the dual AAVs.

A “hybrid AAV” or “hybrid rAAV” refers to an rAAV genome packaged with a capsid of a different AAV serotype (and preferably, of a different serotype from the one or more AAV ITRs), and may otherwise be referred to as a pseudotyped rAAV. For example, an rAAV type 1, 2, 3, 4, 5, 6, 7, 8, 9, 10, 11, 12, 13, 14, 15, or 16 genome may be encapsidated within an AAV type 1, 2, 3, 4, 5, 6, 7, 8, 9, 10, 11, 12, 13, 14, 15, or 16 capsid or variants thereof, provided that the AAV capsid and genome (and preferably, the one or more AAV ITRs) are of different serotypes. In certain embodiments, a pseudotyped rAAV particle may be referred to as being of the type “x/y”, where “x” indicates the source of ITRs and “y” indicates the serotype of capsid, for example a 2/5 rAAV particle has ITRs from AAV2 and a capsid from AAV6.

A “host cell” includes cells transfected, infected, or transduced in vivo, ex vivo, or in vitro with a recombinant vector or a polynucleotide of the disclosure. Host cells may include virus producing cells and cells infected with viral vectors. In particular embodiments, host cells in vivo are infected with viral vector contemplated herein. In certain embodiments, the term “target cell” is used interchangeably with host cell and refers to infected cells of a desired cell type.

High titer AAV preparations can be produced using techniques known in the art, e.g., as described in U.S. Pat. Nos. 5,658,776; 6,566,118; 6,989,264; and 6,995,006; U.S. 2006/0188484; WO98/22607; WO2005/072364; and WO/1999/011764; and Viral Vectors for Gene Therapy: Methods and Protocols, ed. Machida, Humana Press, 2003; Samulski et al., (1989) J. Virology 63, 3822; Xiao et al., (1998) J. Virology 72, 2224; lnoue et al., (1998) J. Virol. 72, 7024. Methods of producing pseudotyped AAV vectors have also been reported (e.g., WO 00/28004), as well as various modifications or formulations of AAV vectors, to reduce their immunogenicity upon in vivo administration (see e.g., WO 01/23001; WO 00/73316; WO 04/1 12727; WO 05/005610; WO 99/06562).

### Pharmaceutical Compositions

Also provided are pharmaceutical preparations, including pharmaceutical preparations of vector and pharmaceutical preparations of binding agent. Pharmaceutical preparations include the subject polynucleotide (RNA or DNA) encoding an engineered receptor, vector carrying a polynucleotide (RNA or DNA) encoding a subject engineered receptor, or binding agent present in a pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the disclosure is formulated for administration to a mammal. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a mammal, the compounds and compositions of the disclosure and pharmaceutically acceptable vehicles, excipients, or diluents may be sterile. In some instances, an aqueous medium is employed as a vehicle when the compound of the disclosure is administered intravenously, such as water, saline solutions, and aqueous dextrose and glycerol solutions.

Pharmaceutical compositions can take the form of capsules, tablets, pills, pellets, lozenges, powders, granules, syrups, elixirs, solutions, suspensions, emulsions, suppositories, or sustained-release formulations thereof, or any other form suitable for administration to a mammal. In some instances, the pharmaceutical compositions are formulated for administration in accordance with routine procedures as a pharmaceutical composition adapted for oral or intravenous administration to humans. Examples of suitable pharmaceutical vehicles and methods for formulation thereof are described in Remington: The Science and Practice of Pharmacy, Alfonso R. Gennaro ed., Mack Publishing Co. Easton, Pa., 19th ed., 1995, Chapters 86, 87, 88, 91, and 92, incorporated herein by reference.

The choice of excipient will be determined in part by the particular vector, as well as by the particular method used to administer the composition. Accordingly, there is a wide variety of suitable formulations of the pharmaceutical composition of the present disclosure.

For example, the vector may be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

As another example, the vector may be formulated into a preparation suitable for oral administration, including (a) liquid solutions, such as an effective amount of the compound dissolved in diluents, such as water, or saline; (b) capsules, sachets or tablets, each containing a predetermined amount of the active ingredient, as solids or granules; (c) suspensions in an appropriate liquid; and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can include the active ingredient in a flavor, usually sucrose and acacia or tragacanth, as well as pastilles including the active ingredient in an inert base, such as gelatin and glycerin, or sucrose and acacia, emulsions, gels, and the like containing, in addition to the active ingredient, such excipients as are described herein.

As another example, the subject formulations of the present disclosure can be made into aerosol formulations to be administered via inhalation. These aerosol formulations can be placed into pressurized acceptable propellants, such as dichlorodifluoromethane, propane, nitrogen, and the like. They may also be formulated as pharmaceuticals for non-pressured preparations such as for use in a nebulizer or an atomizer.

In some embodiments, formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Formulations suitable for topical administration may be presented as creams, gels, pastes, or foams, containing, in addition to the active ingredient, such carriers as are appropriate. In some embodiments the topical formulation contains one or more components selected from a structuring agent, a thickener or gelling agent, and an emollient or lubricant. Frequently employed structuring agents include long chain alcohols, such as stearyl alcohol, and glyceryl ethers or esters and oligo(ethylene oxide) ethers or esters thereof. Thickeners and gelling agents include, for example, polymers of acrylic or methacrylic acid and esters thereof, polyacrylamides, and naturally occurring thickeners such as agar, carrageenan, gelatin, and guar gum. Examples of emollients include triglyceride esters, fatty acid esters and amides, waxes such as beeswax, spermaceti, or carnauba wax, phospholipids such as lecithin, and sterols and fatty acid esters thereof. The topical formulations may further include other components, e.g., astringents, fragrances, pigments, skin penetration enhancing agents, sunscreens (i.e., sunblocking agents), etc.

A compound of the disclosure may be formulated for topical administration. The vehicle for topical application may be in one of various forms, e.g. a lotion, cream, gel, ointment, stick, spray, or paste. They may contain various types of carriers, including, but not limited to, solutions, aerosols, emulsions, gels, and liposomes. The carrier may be formulated, for example, as an emulsion, having an oil-in-water or water-in-oil base. Suitable hydrophobic (oily) components employed in emulsions include, for example, vegetable oils, animal fats and oils, synthetic hydrocarbons, and esters and alcohols thereof, including polyesters, as well as organopolysiloxane oils. Such emulsions also include an emulsifier and/or surfactant, e.g. a nonionic surfactant to disperse and suspend the discontinuous phase within the continuous phase.

Suppository formulations are also provided by mixing with a variety of bases such as emulsifying bases or water-soluble bases. Formulations suitable for vaginal administration may be presented as pessaries, tampons, creams, gels, pastes, foams.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may include the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present disclosure calculated in an amount sufficient to produce the desired effect in association with a pharmaceutically acceptable diluent, carrier or vehicle. The specifications for the novel unit dosage forms of the present disclosure depend on the particular compound employed and the effect to be achieved, and the pharmacodynamics associated with each compound in the host.

Dose levels can vary as a function of the specific compound, the nature of the delivery vehicle, and the like. Desired dosages for a given compound are readily determinable by a variety of means.

The dose administered to an animal, particularly a human, in the context of the present disclosure should be sufficient to effect a prophylactic or therapeutic response in the animal over a reasonable time frame, e.g., as described in greater detail below. Dosage will depend on a variety of factors including the strength of the particular compound employed, the condition of the animal, and the body weight of the animal, as well as the severity of the illness and the stage of the disease. The size of the dose will also be determined by the existence, nature, and extent of any adverse side-effects that might accompany the administration of a particular compound.

In pharmaceutical dosage forms, the ASC inducer compounds may be administered in the form of a free base, their pharmaceutically acceptable salts, or they may also be used alone or in appropriate association, as well as in combination, with other pharmaceutically active compounds.

### G. Clinical Applications and Methods of Treatment

The compositions and methods disclosed herein can be utilized to treat a neurological disease or disorder. In some aspects of the disclosure, a method of treating a neurological disease or disorder in a subject is provided, the method comprising the introducing an engineered receptor into a neuronal cell and providing a ligand that activates the engineered receptor in an effective amount to control the activity of the cell, thereby relieving pain in the subject. In some aspects, vectors or compositions disclosed herein are used in the manufacture of a medicament for treating a neurological disease or disorder.

In some cases, the methods and compositions of the disclosure are utilized to treat epilepsy. Compositions described herein may be used to prevent or control epileptic seizures. Epileptic seizures may be classified as tonic-clonic, tonic, clonic, myoclonic, absence or atonic seizures. In some cases, the compositions and methods herein may prevent or reduce the number of epileptic seizures experienced by a subject by about 5%, about 10%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or 100%.

In some cases, the methods and compositions of the disclosure are utilized to treat an eating disorder. An eating disorder may be a mental disorder defined by abnormal eating behaviors that negatively affect a subject's physical or mental health. In some cases, the eating disorder is anorexia nervosa. In other cases, the eating disorder is bulimia nervosa. In some cases, the eating disorder is pica, rumination disorder, avoidant/restrictive food intake disorder, binge eating disorder (BED), other specified feeding and eating disorder (OSFED), compulsive overeating, diabulimia, orthorexia nervosa, selective eating disorder, drunkorexia, pregorexia, or Gourmand syndrome. In some cases, the composition includes a G-protein coupled receptor that increases or decreases the production of one or more molecules associated with an eating disorder. In other cases, the composition includes a ligand-gated ion channel that alters the production of one or more molecules associated with an eating disorder. The one or more molecules associated with an eating disorder may include, without limitation, a molecule of the hypothalamus-pituitary-adrenal (HPA) axis, including vasopressin, corticotropin-releasing hormone (CRH), adrenocorticotropic hormone (ACTH), cortisol, epinephrine, or norepinephrine; as well as serotonin, dopamine, neuropeptide Y, leptin, or ghrelin.

In some cases, the compositions and methods are utilized to treat post-traumatic stress disorder (PTSD), gastroesophageal reflex disease (GERD), addiction (e.g., alcohol, drugs), anxiety, depression, memory loss, dementia, sleep apnea, stroke, urinary incontinence, narcolepsy, essential tremor, movement disorder, atrial fibrillation, cancer (e.g., brain tumors), Parkinson's disease, or Alzheimer's disease. Other non-limiting examples of neurological diseases or disorders that can be treated by the compositions and methods herein include: Abulia, Agraphia, Alcoholism, Alexia, Aneurysm, Amaurosis fugax, Amnesia, Amyotrophic lateral sclerosis (ALS), Angelman syndrome, Aphasia, Apraxia, Arachnoiditis, Arnold-Chiari malformation, Asperger syndrome, Ataxia, Ataxia-telangiectasia, Attention deficit hyperactivity disorder, Auditory processing disorder, Autism spectrum, Bipolar disorder, Bell's palsy, Brachial plexus injury, Brain damage, Brain injury, Brain tumor, Canavan disease, Capgras delusion, Carpal tunnel syndrome, Causalgia, Central pain syndrome, Central pontine myelinolysis, Centronuclear myopathy, Cephalic disorder, Cerebral aneurysm, Cerebral arteriosclerosis, Cerebral atrophy, Cerebral autosomal dominant arteriopathy with subcortical infarcts and leukoencephalopathy (CADASIL), Cerebral gigantism, Cerebral palsy, Cerebral vasculitis, Cervical spinal stenosis, Charcot-Marie-Tooth disease, Chiari malformation, Chorea, Chronic fatigue syndrome, Chronic inflammatory demyelinating polyneuropathy (CIDP), Chronic pain, Coffin-Lowry syndrome, Coma, Complex regional pain syndrome, Compression neuropathy, Congenital facial diplegia, Corticobasal degeneration, Cranial arteritis, Craniosynostosis, Creutzfeldt-Jakob disease, Cumulative trauma disorders, Cushing's syndrome, Cyclothymic disorder, Cytomegalic inclusion body disease (CIBD), Cytomegalovirus Infection, Dandy-Walker syndrome, Dawson disease, De Morsier's syndrome, Dejerine-Klumpke palsy, Dejerine-Sottas disease, Delayed sleep phase syndrome, Dementia, Dermatomyositis, Developmental coordination disorder, Diabetic neuropathy, Diffuse sclerosis, Diplopia, Down syndrome, Dravet syndrome, Duchenne muscular dystrophy, Dysarthria, Dysautonomia, Dyscalculia, Dysgraphia, Dyskinesia, Dyslexia, Dystonia, Empty sella syndrome, Encephalitis, Encephalocele, Encephalotrigeminal angiomatosis, Encopresis, Enuresis, Epilepsy, Epilepsy-intellectual disability in females, Erb's palsy, Erythromelalgia, Exploding head syndrome, Fabry's disease, Fahr's syndrome, Fainting, Familial spastic paralysis, Febrile seizures, Fisher syndrome, Friedreich's ataxia, Fibromyalgia, Foville's syndrome, Fetal alcohol syndrome, Fragile X syndrome, Fragile X-associated tremor/ataxia syndrome (FXTAS), Gaucher's disease, Generalized epilepsy with febrile seizures plus, Gerstmann's syndrome, Giant cell arteritis, Giant cell inclusion disease, Globoid Cell Leukodystrophy, Gray matter heterotopia, Guillain-Barré syndrome, Generalized anxiety disorder, HTLV-1 associated myelopathy, Hallervorden-Spatz disease, Head injury, Headache, Hemifacial Spasm, Hereditary Spastic Paraplegia, Heredopathia atactica polyneuritiformis, Herpes zoster oticus, Herpes zoster, Hirayama syndrome, Hirschsprung's disease, Holmes-Adie syndrome, Holoprosencephaly, Huntington's disease, Hydranencephaly, Hydrocephalus, Hypercortisolism, Hypoxia, Immune-Mediated encephalomyelitis, Inclusion body myositis, Incontinentia pigmenti, Infantile Refsum disease, Infantile spasms, Inflammatory myopathy, Intracranial cyst, Intracranial hypertension, Isodicentric 15, Joubert syndrome, Karak syndrome, Kearns-Sayre syndrome, Kinsbourne syndrome, Kleine-Levin Syndrome, Klippel Feil syndrome, Krabbe disease, Lafora disease, Lambert-Eaton myasthenic syndrome, Landau-Kleffner syndrome, Lateral medullary (Wallenberg) syndrome, Learning disabilities, Leigh's disease, Lennox-Gastaut syndrome, Lesch-Nyhan syndrome, Leukodystrophy, Leukoencephalopathy with vanishing white matter, Lewy body dementia, Lissencephaly, Locked-In syndrome, Lumbar disc disease, Lumbar spinal stenosis, Lyme disease—Neurological Sequelae, Machado-Joseph disease (Spinocerebellar ataxia type 3), Macrencephaly, Macropsia, Mal de debarquement, Megalencephalic leukoencephalopathy with subcortical cysts, Megalencephaly, Melkersson-Rosenthal syndrome, Menieres disease, Meningitis, Menkes disease, Metachromatic leukodystrophy, Microcephaly, Micropsia, Migraine, Miller Fisher syndrome, Mini-stroke (transient ischemic attack), Misophonia, Mitochondrial myopathy, Mobius syndrome, Monomelic amyotrophy, Motor skills disorder, Moyamoya disease, Mucopolysaccharidoses, Multi-infarct dementia, Multifocal motor neuropathy, Multiple sclerosis, Multiple system atrophy, Muscular dystrophy, Myalgic encephalomyelitis, Myasthenia gravis, Myelinoclastic diffuse sclerosis, Myoclonic Encephalopathy of infants, Myoclonus, Myopathy, Myotubular myopathy, Myotonia congenita, Narcolepsy, Neuro-Behcet's disease, Neurofibromatosis, Neuroleptic malignant syndrome, Neurological manifestations of AIDS, Neurological sequelae of lupus, Neuromyotonia, Neuronal ceroid lipofuscinosis, Neuronal migration disorders, Neuropathy, Neurosis, Niemann-Pick disease, Non-24-hour sleep-wake disorder, Nonverbal learning disorder, O'Sullivan-McLeod syndrome, Occipital Neuralgia, Occult Spinal Dysraphism Sequence, Ohtahara syndrome, Olivopontocerebellar atrophy, Opsoclonus myoclonus syndrome, Optic neuritis, Orthostatic Hypotension, Otosclerosis, Overuse syndrome, Palinopsia, Paresthesia, Parkinson's disease, Paramyotonia Congenita, Paraneoplastic diseases, Paroxysmal attacks, Parry-Romberg syndrome, PANDAS, Pelizaeus-Merzbacher disease, Periodic Paralyses, Peripheral neuropathy, Pervasive developmental disorders, Photic sneeze reflex, Phytanic acid storage disease, Pick's disease, Pinched nerve, Pituitary tumors, PMG, Polyneuropathy, Polio, Polymicrogyria, Polymyositis, Porencephaly, Post-Polio syndrome, Postherpetic Neuralgia (PHN), Postural Hypotension, Prader-Willi syndrome, Primary Lateral Sclerosis, Prion diseases, Progressive hemifacial atrophy, Progressive multifocal leukoencephalopathy, Progressive Supranuclear Palsy, Prosopagnosia, Pseudotumor cerebri, Quadrantanopia, Quadriplegia, Rabies, Radiculopathy, Ramsay Hunt syndrome type I, Ramsay Hunt syndrome type II, Ramsay Hunt syndrome type III, Rasmussen encephalitis, Reflex neurovascular dystrophy, Refsum disease, REM sleep behavior disorder, Repetitive stress injury, Restless legs syndrome, Retrovirus-associated myelopathy, Rett syndrome, Reye's syndrome, Rhythmic Movement Disorder, Romberg syndrome, Saint Vitus dance, Sandhoff disease, Schilder's disease, Schizencephaly, Sensory processing disorder, Septo-optic dysplasia, Shaken baby syndrome, Shingles, Shy-Drager syndrome, Sjögren's syndrome, Sleep apnea, Sleeping sickness, Snatiation, Sotos syndrome, Spasticity, Spina bifida, Spinal cord injury, Spinal cord tumors, Spinal muscular atrophy, Spinal and bulbar muscular atrophy, Spinocerebellar ataxia, Split-brain, Steele-Richardson-Olszewski syndrome, Stiff-person syndrome, Stroke, Sturge-Weber syndrome, Stuttering, Subacute sclerosing panencephalitis, Subcortical arteriosclerotic encephalopathy, Superficial siderosis, Sydenham's chorea, Syncope, Synesthesia, Syringomyelia, Tarsal tunnel syndrome, Tardive dyskinesia, Tardive dysphrenia, Tarlov cyst, Tay-Sachs disease, Temporal arteritis, Temporal lobe epilepsy, Tetanus, Tethered spinal cord syndrome, Thomsen disease, Thoracic outlet syndrome, Tic Douloureux, Todd's paralysis, Tourette syndrome, Toxic encephalopathy, Transient ischemic attack, Transmissible spongiform encephalopathies, Transverse myelitis, Traumatic brain injury, Tremor, Trichotillomania, Trigeminal neuralgia, Tropical spastic paraparesis, Trypanosomiasis, Tuberous sclerosis, Unverricht-Lundborg disease, Von Hippel-Lindau disease (VHL), Viliuisk Encephalomyelitis (VE), Wallenberg's syndrome, West syndrome, Whiplash, Williams syndrome, Wilson's disease, or Zellweger syndrome.

In some cases, the compositions and methods disclosed herein can be used to treat brain cancer or brain tumors. Non-limiting examples of brain cancers or tumors that may be amenable to treatment with vectors and compositions described herein include: gliomas including anaplastic astrocytoma (grade III glioma), astrocytoma (grade II glioma), brainstem glioma, ependymoma, ganglioglioma, ganglioneuroma, glioblastoma (grade IV glioma), glioma, juvenile pilocytic astrocytoma (JPA), low-grade astrocytoma (LGA), medullablastoma, mixed glioma, oligodendroglioma, optic nerve glioma, pilocytic astrocytoma (grade I glioma), and primitive neuroectodermal (PNET); skull base tumors including acoustic neuroma (vestibular schwannoma), acromegaly, adenoma, chondrosarcoma, chordoma, craniopharyngioma, epidermoid tumor, glomus jugulare tumor, infratentorial meningioma, meningioma, pituitary adenoma, pituitary tumor, Rathke's cleft cyst; metastatic cancer including brain metastasis, metastatic brain tumor; other brain tumors including brain cyst, choroid plexus papilloma, CNS lymphoma, colloid cyst, cystic tumor, dermoid tumor, germinoma, lymphoma, nasal carcinoma, naso-pharyngeal tumor, pineal tumor, pineoblastoma, pineocytoma, supratentorial meningioma, and vascular tumor; spinal cord tumors including astrocytoma, ependymoma, meningioma, and schwannoma.

The present disclosure contemplates, in part, compositions and methods for controlling, managing, preventing, or treating pain in a subject. “Pain” refers to an uncomfortable feeling and/or an unpleasant sensation in the body of a subject. Feelings of pain can range from mild and occasional to severe and constant. Pain can be classified as acute pain or chronic pain. Pain can be nociceptive pain (i.e., pain caused by tissue damage), neuropathic pain or psychogenic pain. In some cases, the pain is caused by or associated with a disease (e.g., cancer, arthritis, diabetes). In other cases, the pain is caused by injury (e.g., sports injury, trauma). Non-limiting examples of pain that are amenable to treatment with the compositions and methods herein include: neuropathic pain including peripheral neuropathy, diabetic neuropathy, post herpetic neuralgia, trigeminal neuralgia, back pain, neuropathy associated with cancer, neuropathy associated with HIV/AIDS, phantom limb pain, carpal tunnel syndrome, central post-stroke pain, pain associated with chronic alcoholism, hypothyroidism, uremia, pain associated with multiple sclerosis, pain associated with spinal cord injury, pain associated with Parkinson's disease, epilepsy, osteoarthritic pain, rheumatoid arthritic pain, visceral pain, and pain associated with vitamin deficiency; and nociceptive pain including pain associated with central nervous system trauma, strains/sprains, and burns; myocardial infarction, acute pancreatitis, post-operative pain, posttraumatic pain, renal colic, pain associated with cancer, pain associated with fibromyalgia, pain associated with carpal tunnel syndrome, and back pain.

The compositions and methods herein may be utilized to ameliorate a level of pain in a subject. In some cases, a level of pain in a subject is ameliorated by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least 99% or about 100%. A level of pain in a subject can be assessed by a variety of methods. In some cases, a level of pain is assessed by self-reporting (i.e., a human subject expresses a verbal report of the level of pain he/she is experiencing). In some cases, a level of pain is assessed by behavioral indicators of pain, for example, facial expressions, limb movements, vocalization, restlessness and guarding. These types of assessments may be useful for example when a subject is unable to self-report (e.g., an infant, an unconscious subject, a non-human subject). A level of pain may be assessed after treatment with a composition of the disclosure as compared to the level of pain the subject was experiencing prior to treatment with the composition.

In various embodiments, a method for controlling, managing, preventing, or treating pain in a subject comprises administering to the subject an effective amount of an engineered receptor contemplated herein. Without wishing to be bound by any particular theory, the present disclosure contemplates using the vectors disclosed herein to modulate neuronal activity to alleviate pain in the subject.

In various embodiments, a vector encoding an engineered receptor that activates or depolarizes neuronal cells is administered to (or introduced into) one or more neuronal cells that decrease pain sensation, e.g., inhibitory interneurons. In the presence of ligand the neuronal cell expressing the engineered receptor, is activated and decreases the sensitivity to pain potentiating the analgesic effect of stimulating these neuronal cells.

In various embodiments, a vector encoding an engineered receptor that deactivates or hyperpolarizes neuronal cells is administered to (or introduced into) one or more neuronal cells that increase pain sensation or sensitivity to pain, e.g., nociceptor, peripheral sensory neurons, C-fibers, Aδ fibers, Aβ fibers, DRG neurons, TGG neurons, and the like. In the presence of ligand the neuronal cell expressing the engineered receptor, is deactivated and decreases the sensitivity to pain and potentiating an analgesic effect.

Targeting expression of an engineered receptor to a sub-population of nociceptors can be achieved by one or more of: selection of the vector (e.g., AAV1, AAV1(Y705+731F+T492V), AAV2(Y444+500+730F+T491V), AAV3(Y705+731F), AAV5, AAV5(Y436+693+719F), AAV6, AAV6 (VP3 variant Y705F/Y731F/T492V), AAV-7m8, AAV8, AAV8(Y733F), AAV9, AAV9 (VP3 variant Y731F), AAV10(Y733F), and AAV-ShH10); selection of a promoter; and delivery means.

In particular embodiments, the compositions and methods contemplated herein are effective in reducing pain. Illustrative examples of pain that are amenable to treatment with the vectors, compositions, and methods contemplated herein, include but are not limited to acute pain, chronic pain, neuropathic pain, nociceptive pain, allodynia, inflammatory pain, inflammatory hyperalgesia, neuropathies, neuralgia, diabetic neuropathy, human immunodeficiency virus-related neuropathy, nerve injury, rheumatoid arthritic pain, osteoarthritic pain, burns, back pain, eye pain, visceral pain, cancer pain (e.g., bone cancer pain), dental pain, headache, migraine, carpal tunnel syndrome, fibromyalgia, neuritis, sciatica, pelvic hypersensitivity, pelvic pain, post herpetic neuralgia, post-operative pain, post stroke pain, and menstrual pain.

Pain can be classified as acute or chronic. “Acute pain” refers to pain that begins suddenly and is usually sharp in quality. Acute pain might be mild and last just a moment, or it might be severe and last for weeks or months. In most cases, acute pain does not last longer than three months, and it disappears when the underlying cause of pain has been treated or has healed. Unrelieved acute pain, however, may lead to chronic pain. “Chronic pain” refers to ongoing or recurrent pain, lasting beyond the usual course of acute illness or injury or lasting for more than three to six months, and which adversely affects the individual's well-being. In particular embodiments, the term “chronic pain” refers to pain that continues when it should not. Chronic pain can be nociceptive pain or neuropathic pain.

In some embodiments, the pain is expected or anticipated to develop in association with or as a result of an injury, an infection, or a medical intervention. In some embodiments, the infection causes nerve damage. In some embodiments, the medical intervention is a surgery, such as surgery to the central core of the body. In some embodiments, the medical intervention is a surgery to remove parts or whole of one or more tissues, tumors or organs in the body. In some embodiments, the medical intervention is an amputation. In particular embodiments, the compositions and methods contemplated herein are effective in reducing acute pain. In particular embodiments, the compositions and methods contemplated herein are effective in reducing chronic pain.

Clinical pain is present when discomfort and abnormal sensitivity feature among the patient's symptoms. Individuals can present with various pain symptoms. Such symptoms include: 1) spontaneous pain which may be dull, burning, or stabbing; 2) exaggerated pain responses to noxious stimuli (hyperalgesia); and 3) pain produced by normally innocuous stimuli (allodynia-Meyer et al., 1994, Textbook of Pain, 13-44). Although patients suffering from various forms of acute and chronic pain may have similar symptoms, the underlying mechanisms may be different and may, therefore, require different treatment strategies. Pain can also therefore be divided into a number of different subtypes according to differing pathophysiology, including nociceptive pain, inflammatory pain, and neuropathic pain.

In particular embodiments, the compositions and methods contemplated herein are effective in reducing nociceptive pain. In particular embodiments, the compositions and methods contemplated herein are effective in reducing inflammatory pain. In particular embodiments, the compositions and methods contemplated herein are effective in reducing neuropathic pain.

Nociceptive pain is induced by tissue injury or by intense stimuli with the potential to cause injury. Moderate to severe acute nociceptive pain is a prominent feature of pain from central nervous system trauma, strains/sprains, burns, myocardial infarction and acute pancreatitis, post-operative pain (pain following any type of surgical procedure), posttraumatic pain, renal colic, cancer pain and back pain. Cancer pain may be chronic pain such as tumor related pain (e.g., bone pain, headache, facial pain or visceral pain) or pain associated with cancer therapy (e.g., post chemotherapy syndrome, chronic postsurgical pain syndrome or post radiation syndrome). Cancer pain may also occur in response to chemotherapy, immunotherapy, hormonal therapy or radiotherapy. Back pain may be due to herniated or ruptured intervertebral discs or abnormalities of the lumber facet joints, sacroiliac joints, paraspinal muscles or the posterior longitudinal ligament. Back pain may resolve naturally but in some patients, where it lasts over 12 weeks, it becomes a chronic condition which can be particularly debilitating.

Neuropathic pain can be defined as pain initiated or caused by a primary lesion or dysfunction in the nervous system. Etiologies of neuropathic pain include, e.g., peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy, and vitamin deficiency.

Neuropathic pain can be related to a pain disorder, a term referring to a disease, disorder or condition associated with or caused by pain. Illustrative examples of pain disorders include arthritis, allodynia, a typical trigeminal neuralgia, trigeminal neuralgia, somatoform disorder, hypoesthesis, hypealgesia, neuralgia, neuritis, neurogenic pain, analgesia, anesthesia dolorosa, causlagia, sciatic nerve pain disorder, degenerative joint disorder, fibromyalgia, visceral disease, chronic pain disorders, migraine/headache pain, chronic fatigue syndrome, complex regional pain syndrome, neurodystrophy, plantar fasciitis or pain associated with cancer.

The inflammatory process is a complex series of biochemical and cellular events, activated in response to tissue injury or the presence of foreign substances, which results in swelling and pain. Arthritic pain is a common inflammatory pain.

Other types of pain that are amenable to treatment with the vectors, compositions, and methods contemplated herein, include but are not limited to pain resulting from musculoskeletal disorders, including myalgia, fibromyalgia, spondylitis, sero-negative (non-rheumatoid) arthropathies, non-articular rheumatism, dystrophinopathy, glycogenolysis, polymyositis and pyomyositis; heart and vascular pain, including pain caused by angina, myocardical infarction, mitral stenosis, pericarditis, Raynaud's phenomenon, scleredoma and skeletal muscle ischemia; head pain, such as migraine (including migraine with aura and migraine without aura), cluster headache, tension-type headache mixed headache and headache associated with vascular disorders; and orofacial pain, including dental pain, otic pain, burning mouth syndrome, and temporomandibular myofascial pain.

The effective amount of the compositions and methods contemplated herein to reduce the amount of pain experienced by a human subject can be determined using a variety of pain scales. Patient self-reporting can be used to assess whether pain is reduced; see, e.g., Katz and Melzack (1999) Surg. Clin. North Am. 79:231. Alternatively, an observational pain scale can be used. The LANSS Pain Scale can be used to assess whether pain is reduced; see, e.g., Bennett (2001) Pain 92:147. A visual analog pain scale can be used; see, e.g., Schmader (2002) Clin. J. Pain 18:350. The Likert pain scale can be used; e.g., where 0 is no pain, 5 is moderate pain, and 10 is the worst pain possible. Self-report pain scales for children include, e.g., Faces Pain Scale; Wong-Baker FACES Pain Rating Scale; and Colored Analog Scale. Self-report pain scales for adults include, e.g., Visual Analog Scale; Verbal Numerical Rating Scale; Verbal Descriptor Scale; and Brief Pain Inventory. Pain measurement scales include, e.g., Alder Hey Triage Pain Score (Stewart et al. (2004) Arch. Dis. Child. 89:625); Behavioral Pain Scale (Payen et al. (2001) Critical Care Medicine 29:2258); Brief Pain Inventory (Cleeland and Ryan (1994) Ann. Acad. Med. Singapore 23: 129); Checklist of Nonverbal Pain Indicators (Feldt (2000) Pain Manag. Nurs. 1: 13); Critical-Care Pain Observation Tool (Gelinas et al. (2006) Am. J. Crit. Care 15:420); COMFORT scale (Ambuel et al. (1992) J. Pediatric Psychol. 17:95); Dallas Pain Questionnaire (Ozguler et al. (2002) Spine 27:1783); Dolorimeter Pain Index (Hardy et al. (1952) Pain Sensations and Reactions Baltimore: The Williams & Wilkins Co.); Faces Pain Scale—Revised (Hicks et al. (2001) Pain 93:173); Face Legs Activity Cry Consolability Scale; McGill Pain Questionnaire (Melzack (1975) Pain 1:277); Descriptor Differential Scale (Gracely and Kwilosz (1988) Pain 35:279); Numerical 1 1 point Box (Jensen et al. (1989) Clin. J. Pain 5: 153); Numeric Rating Scale (Hartrick et al. (2003) Pain Pract. 3:310); Wong-Baker FACES Pain Rating Scale; and Visual Analog Scale (Huskisson (1982) J. Rheumatol. 9:768).

In particular embodiments, a method of relieving pain in a subject is provided, the method comprising introducing an engineered receptor into a neuronal cell and controlling the activity of the cell by providing an effective amount of a ligand that activates the engineered receptor, thereby relieving pain in the subject. The method provides significant analgesia for pain without off-target effects, such as general central nervous system depression. In certain embodiments, the method provides a 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more reduction in the neuropathic pain in a subject compared to an untreated subject. In some embodiments, the method comprises the step of measuring pain in the subject before and after the administration of the binding agent, wherein the pain in the subject is reduced 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or more. In such instances, the measuring may occur 4 hours or more after administration of the binding agent, e.g. 8 hours 12 hours, 16 hours, 24 hours, 36 hours, 48 hours, 3 days, or 4 days or more after administration of the binding agent.

In particular embodiments, the vectors contemplated herein are administered or introduced into one or more neuronal cells. The neuronal cells may be the same type of neuronal cells, or a mixed population of different types of neuronal cells. In one embodiment, the neuronal cell is a nociceptor or peripheral sensory neuron. Illustrative examples of sensory neurons include, but are not limited to, dorsal root ganglion (DRG) neurons and trigeminal ganglion (TGG) neurons. In one embodiment, the neuronal cell is an inhibitory interneuron involved in the neuronal pain circuit.

In some cases, a vector encoding an engineered receptor is administered to a subject in need thereof. Non-limiting examples of methods of administration include subcutaneous administration, intravenous administration, intramuscular administration, intradermal administration, intraperitoneal administration, oral administration, infusion, intracranial administration, intrathecal administration, intranasal administration, intraganglionic administration, intraspinal administration, cisterna magna administration and intraneural administration. In some cases, administration can involve injection of a liquid formulation of the vector. In other cases, administration can involve oral delivery of a solid formulation of the vector. In some cases, the oral formulation can be administered with food. In particular embodiments, a vector is parenterally, intravenously, intramuscularly, intraperitoneally, intrathecally, intraneurally, intraganglionicly, intraspinally, or intraventricularly administered to a subject in order to introduce the vector into one or more neuronal cells. In various embodiments, the vector is rAAV.

In one embodiment, AAV is administered to sensory neuron or nociceptor, e.g., DRG neurons, TGG neurons, etc. by intrathecal (IT) or intraganglionic (IG) administration. The IT route delivers AAV to the cerebrospinal fluid (CSF). This route of administration may be suitable for the treatment of e.g., chronic pain or other peripheral nervous system (PNS) or central nervous system (CNS) indications. In animals, IT administration has been achieved by inserting an IT catheter through the cisterna magna and advancing it caudally to the lumbar level. In humans, IT delivery can be easily performed by lumbar puncture (LP), a routine bedside procedure with excellent safety profile.

In a particular case, a vector may be administered to a subject by intraganglionic administration. Intraganglionic administration may involve an injection directly into one or more ganglia. The IG route may deliver AAV directly into the DRG or TGG parenchyma. In animals, IG administration to the DRG is performed by an open neurosurgical procedure that is not desirable in humans because it would require a complicated and invasive procedure. In humans, a minimally invasive, CT imaging-guided technique to safely target the DRG can be used. A customized needle assembly for convection enhanced delivery (CED) can be used to deliver AAV into the DRG parenchyma. In a non-limiting example, a vector of the disclosure may be delivered to one or more dorsal root ganglia and/or trigeminal ganglia for the treatment of chronic pain. In another non-limiting example, a vector of the disclosure may be delivered to the nodose ganglion (vagus nerve) to treat epilepsy.

In yet another particular case, a vector may be administered to the subject by intracranial administration (i.e., directly into the brain). In non-limiting examples of intracranial administration, a vector of the disclosure may be delivered into the cortex of the brain to treat e.g., an epileptic seizure focus, into the paraventricular hypothalamus to treat e.g., a satiety disorder, or into the amygdala central nucleus to treat e.g., a satiety disorder. In another particular case, a vector may be administered to a subject by intraneural injection (i.e., directly into a nerve). The nerve may be selected based on the indication to be treated, for example, injection into the sciatic nerve to treat chronic pain or injection into the vagal nerve to treat epilepsy or a satiety disorder. In yet another particular case, a vector may be administered to a subject by subcutaneous injection, for example, into the sensory nerve terminals to treat chronic pain.

A vector dose may be expressed as the number of vector genome units delivered to a subject. A “vector genome unit” as used herein refers to the number of individual vector genomes administered in a dose. The size of an individual vector genome will generally depend on the type of viral vector used. Vector genomes of the disclosure may be from about 1.0 kilobase, 1.5 kilobases, 2.0 kilobases, 2.5 kilobases, 3.0 kilobases, 3.5 kilobases, 4.0 kilobases, 4.5 kilobases, 5.0 kilobases, 5.5 kilobases, 6.0 kilobases, 6.5 kilobases, 7.0 kilobases, 7.5 kilobases, 8.0 kilobases, 8.5 kilobases, 9.0 kilobases, 9.5 kilobases, 10.0 kilobases, to more than 10.0 kilobases. Therefore, a single vector genome may include up to or greater than 10,000 base pairs of nucleotides. In some cases, a vector dose may be about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰ 9×10¹⁰ 1×10¹¹, 2×10¹¹, 3×10¹¹, 4×10¹¹, 5×10¹¹, 6×10¹¹, 7×10¹¹, 8×10¹¹, 9×10¹¹ 1×10¹² 2×10¹², 3×10¹², 4×10¹², 5×10¹², 6×10¹², 7×10¹², 8×10¹², 9×10¹², 1×10¹³ 2×10¹³ 3×10¹³, 4×10¹³, 5×10¹³, 6×10¹³, 7×10¹³, 8×10¹³, 9×10¹³, 1×10¹⁴, 2×10¹⁴ 3×10¹⁴ 4×10¹⁴, 5×10¹⁴, 6×10¹⁴, 7×10¹⁴, 8×10¹⁴, 9×10¹⁴, 1×10¹⁵, 2×10¹⁵, 3×10¹⁵ 4×10¹⁵ 5×10¹⁵, 6×10¹⁵, 7×10¹⁵, 8×10¹⁵, 9×10¹⁵, 1×10¹⁶, 2×10¹⁶, 3×10¹⁶, 4×10¹⁶ 5×10¹⁶ 6×10¹⁶, 7×10¹⁶, 8×10¹⁶, 9×10¹⁶, 1×10¹⁷, 2×10¹⁷, 3×10¹⁷, 4×10¹⁷, 5×10¹⁷, 6×10¹⁷, 7×10¹⁷, 8×10¹⁷, 9×10¹⁷, 1×10¹⁸, 2×10¹⁸, 3×10¹⁸, 4×10¹⁸, 5×10¹⁸, 6×10¹⁸, 7×10¹⁸, 8×10¹⁸, 9×10¹⁸, 1×10¹⁹, 2×10¹⁹, 3×10¹⁹, 4×10¹⁹, 5×10¹⁹, 6×10¹⁹, 7×10¹⁹, 8×10¹⁹, 9×10¹⁹, 1×10²⁰, 2×10²⁰, 3×10²⁰, 4×10²⁰, 5×10²⁰, 6×10²⁰, 7×10²⁰, 8×10²⁰, 9×10²⁰ or more vector genome units.

In particular embodiments, a vector contemplated herein is administered to a subject at a titer of at least about 1×10⁹ genome particles/mL, at least about 1×10¹⁰ genome particles/mL, at least about 5×10¹⁰ genome particles/mL, at least about 1×10¹¹ genome particles/mL, at least about 5×10¹¹ genome particles/mL, at least about 1×10¹² genome particles/mL, at least about 5×10¹² genome particles/mL, at least about 6×10¹² genome particles/mL, at least about 7×10¹² genome particles/mL, at least about 8×10¹² genome particles/mL, at least about 9×10¹² genome particles/mL, at least about 10×10¹² genome particles/mL, at least about 15×10¹² genome particles/mL, at least about 20×10¹² genome particles/mL, at least about 25×10¹² genome particles/mL, at least about 50×10¹² genome particles/mL, or at least about 100×10¹² genome particles/mL. The terms “genome particles (gp)” or “genome equivalents” or “genome copies” (gc) as used in reference to a viral titer, refer to the number of virions containing the recombinant AAV DNA genome, regardless of infectivity or functionality. The number of genome particles in a particular vector preparation can be measured by well understood methods in the art, for example, quantitative PCR of genomic DNA or for example, in Clark et al. (1999) Hum. Gene Ther., 10:1031-1039; Veldwijk et al. (2002) Mol. Ther., 6:272-278.

A vector of the disclosure may be administered in a volume of fluid. In some cases, a vector may be administered in a volume of about 0.1 mL, 0.2 mL, 0.3 mL, 0.4 mL, 0.5 mL, 0.6 mL, 0.7 mL, 0.8 mL, 0.9 mL, 1.0 mL, 2.0 mL, 3.0 mL, 4.0 mL, 5.0 mL, 6.0 mL, 7.0 mL, 8.0 mL, 9.0 mL, 10.0 mL, 11.0 mL, 12.0 mL, 13.0 mL, 14.0 mL, 15.0 mL, 16.0 mL, 17.0 mL, 18.0 mL, 19.0 mL, 20.0 mL or greater than 20.0 mL. In some cases, a vector dose may be expressed as a concentration or titer of vector administered to a subject. In this case, a vector dose may be expressed as the number of vector genome units per volume (i.e., genome units/volume).

In particular embodiments, a vector contemplated herein is administered to a subject at a titer of at least about 5×10⁹ infectious units/mL, at least about 6×10⁹ infectious units/mL, at least about 7×10⁹ infectious units/mL, at least about 8×10⁹ infectious units/mL, at least about 9×10⁹ infectious units/mL, at least about 1×10¹⁰ infectious units/mL, at least about 1.5×10¹⁰ infectious units/mL, at least about 2×10¹⁰ infectious units/mL, at least about 2.5×10¹⁰ infectious units/mL, at least about 5×10¹⁰ infectious units/mL, at least about 1×10¹¹ infectious units/mL, at least about 2.5×10¹¹ infectious units/mL, at least about 5×10¹¹ infectious units/mL, at least about 1×10¹² infectious units/mL, at least about 2.5×10¹² infectious units/mL, at least about 5×10¹² infectious units/mL, at least about 1×10¹³ infectious units/mL, at least about 5×10¹³ infectious units/mL, at least about 1×10¹⁴ infectious units/mL. The terms “infection unit (iu),” “infectious particle,” or “replication unit,” as used in reference to a viral titer, refer to the number of infectious and replication-competent recombinant AAV vector particles as measured by the infectious center assay, also known as replication center assay, as described, for example, in McLaughlin et al. (1988) J. Virol., 62:1963-1973.

In particular embodiments, a vector contemplated herein is administered to a subject at a titer of at least about 5×10¹⁰ transducing units/mL, at least about 1×10¹¹ transducing units/mL, at least about 2.5×10¹¹ transducing units/mL, at least about 5×10¹¹ transducing units/mL, at least about 1×10¹² transducing units/mL, at least about 2.5×10¹² transducing units/mL, at least about 5×10¹² transducing units/mL, at least about 1×10¹³ transducing units/mL, at least about 5×10¹³ transducing units/mL, at least about 1×10¹⁴ transducing units/mL. The term “transducing unit” (tu)” as used in reference to a viral titer, refers to the number of infectious recombinant AAV vector particles that result in the production of a functional transgene product as measured in functional assays such as described in, for example, in Xiao et al. (1997) Exp. Neurobiol., 144:113-124; or in Fisher et al. (1996) J. Virol., 70:520-532 (LFU assay).

The vector dose will generally be determined by the route of administration. In a particular example, an intraganglionic injection may include from about 1×10⁹ to about 1×10¹³ vector genomes in a volume from about 0.1 mL to about 1.0 mL. In another particular case, an intrathecal injection may include from about 1×10¹⁰ to about 1×10¹⁵ vector genomes in a volume from about 1.0 mL to about 12.0 mL. In yet another particular case, an intracranial injection may include from about 1×10⁹ to about 1×10¹³ vector genomes in a volume from about 0.1 mL to about 1.0 mL. In another particular case, an intraneural injection may include from about 1×10⁹ to about 1×10¹³ vector genomes in a volume from about 0.1 mL to about 1.0 mL. In another particular example, an intraspinal injection may include from about 1×10⁹ to about 1×10¹³ vector genomes in a volume from about 0.1 mL to about 1.0 mL. In yet another particular case, a cisterna magna infusion may include from about 5×10⁹ to about 5×10¹³ vector genomes in a volume from about 0.5 mL to about 5.0 mL. In yet another particular case, a subcutaneous injection may include from about 1×10⁹ to about 1×10¹³ vector genomes in a volume from about 0.1 mL to about 1.0 mL.

In some cases, a vector is delivered to a subject by infusion. A vector dose delivered to a subject by infusion can be measured as a vector infusion rate. Non-limiting examples of vector infusion rates include: 1-10 μL/min for intraganglionic, intraspinal, intracranial or intraneural administration; and 10-1000 μL/min for intrathecal or cisterna magna administration. In some cases, the vector is delivered to a subject by MRI-guided Convection Enhanced Delivery (CED). This technique enables increased viral spread and transduction distributed throughout large volumes of the brain, as well as reduces reflux of the vector along the needle path.

In various embodiments, a method is provided comprising administering a vector encoding a engineered receptor, that deactivates or hyperpolarizes neuronal cells, to one or more neuronal cells that increase pain sensation or sensitivity to pain, and administering a ligand that specifically binds the neuronal cell expressing the engineered receptor to the subject, thereby deactivating the cell, decreasing the sensitivity to pain and potentiating an analgesic effect.

In various embodiments, a method is provided comprising administering a vector encoding a engineered receptor, that activates or polarizes neuronal cells, to one or more neuronal cells that decrease pain sensation or sensitivity to pain, and administering a ligand that specifically binds the neuronal cell expressing the engineered receptor to the subject, thereby activating the cell, decreasing the sensitivity to pain and potentiating an analgesic effect.

Formulations of ligands may be administered to a subject by various routes. Non-limiting examples of methods of administration include subcutaneous administration, intravenous administration, intramuscular administration, transdermal administration, intradermal administration, intraperitoneal administration, oral administration, infusion, intracranial administration, intrathecal administration, intranasal administration, intraganglionic administration, and intraneural administration. In some cases, administration can involve injection of a liquid formulation of the ligand. In other cases, administration can involve oral delivery of a solid formulation of the ligand. In a particular case, a ligand is administered by oral administration (e.g., a pill, tablet, capsule and the like). In some cases, the oral composition can be administered with food. In another particular case, a ligand is administered by intrathecal injection (i.e., into the subarachnoid space of the spinal cord) for delivery to the cerebrospinal fluid (CSF) of the subject. In another particular case, a ligand is administered topically (e.g., dermal patch, cream, lotion, ointment and the like).

The dosages of the ligands administered to a subject are not subject to absolute limits, but will depend on the nature of the composition and its active ingredients and its unwanted side effects (e.g., immune response against the antibody), the subject being treated and the type of condition being treated and the manner of administration. Generally, the dose will be a therapeutically effective amount, such as an amount sufficient to achieve a desired biological effect, for example an amount that is effective to decrease or attenuate the level of pain experienced by the subject. In particular embodiments, the dose can also be a prophylactic amount or an effective amount. A therapeutically effective amount of ligand may depend on the route of administration, the indication being treated, and/or the ligand selected for use.

In one embodiment, the ligand is first administered to the subject prior to administration of the vector. A therapeutically effective amount of ligand may be administered to a subject at some time after delivery of a vector. Generally, after delivery of a vector, there will be a period of time required for one or more cells of the subject to generate a protein (i.e., engineered receptor) encoded by the vector. During this period of time, administration of a ligand to the subject may not be beneficial to the subject. In this situation, it may be suitable to administer the ligand after an amount of engineered receptor has been produced by one or more cells of the subject.

In one embodiment, the ligand is first administered to the subject at about the same time that the vector is administered to the subject.

In one embodiment, the ligand is first administered 1, 2, 3, 4, 5, 6, 7, 8, 9, 11, or 12 hours, days, weeks, months, or years after administration of the vector to the subject. In some cases, a therapeutically effective amount of a ligand may be administered to a subject at least one day, two days, three days, four days, five days, six days, seven days, eight days, nine days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more than 30 days after delivery of the vector. In a particular example, a therapeutically effective amount of a ligand is administered to a subject at least one week after delivery of a vector. In a further example, the therapeutically effective amount of ligand is administered to the subject daily for at least three consecutive days.

A therapeutically effective amount or dose of a ligand of the disclosure can be expressed as mg or μg of the ligand per kg of subject body mass. In some instances, a therapeutically effective amount of a ligand may be about 0.001 μg/kg, about 0.005 μg/kg, about 0.01 μg/kg, about 0.05 μg/kg, about 0.1 μg/kg, about 0.5 μg/kg, about 1 μg/kg, about 2 μg/kg, about 3 μg/kg, about 4 μg/kg, about 5 μg/kg, about 6 μg/kg, about 7 μg/kg, about 8 μg/kg, about 9 μg/kg, about 10 μg/kg, about 20 μg/kg, about 30 μg/kg, about 40 μg/kg, about 50 μg/kg, about 60 μg/kg, about 70 μg/kg, about 80 μg/kg, about 90 μg/kg, about 100 μg/kg, about 120 μg/kg, about 140 μg/kg, about 160 μg/kg, about 180 μg/kg, about 200 μg/kg, about 220 μg/kg, about 240 μg/kg, about 260 μg/kg, about 280 μg/kg, about 300 μg/kg, about 320 μg/kg, about 340 μg/kg, about 360 μg/kg, about 380 μg/kg, about 400 μg/kg, about 420 μg/kg, about 440 μg/kg, about 460 μg/kg, about 480 μg/kg, about 500 μg/kg, about 520 μg/kg, about 540 μg/kg, about 560 μg/kg, about 580 μg/kg, about 600 μg/kg, about 620 μg/kg, about 640 μg/kg, about 660 μg/kg, about 680 μg/kg, about 700 μg/kg, about 720 μg/kg, about 740 μg/kg, about 760 μg/kg, about 780 μg/kg, about 800 μg/kg, about 820 μg/kg, about 840 μg/kg, about 860 μg/kg, about 880 μg/kg, about 900 μg/kg, about 920 μg/kg, about 940 μg/kg, about 960 μg/kg, about 980 μg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, or greater than 10 mg/kg.

In particular embodiments, the dose of ligand administered to a subject is at least about 0.001 micrograms per kilogram (μg/kg), at least about 0.005 μg/kg, at least about 0.01 μg/kg, at least about 0.05 μg/kg, at least about 0.1 μg/kg, at least about 0.5 μg/kg, 0.001 milligrams per kilogram (mg/kg), at least about 0.005 mg/kg, at least about 0.01 mg/kg, at least about 0.05 mg/kg, at least about 0.1 mg/kg, at least about 0.5 mg/kg, at least about 1 mg/kg, at least about 2 mg/kg, at least about 3 mg/kg, at least about 4 mg/kg, at least about 5 mg/kg, at least about 5 mg/kg, at least about 6 mg/kg, at least about 7 mg/kg, at least about 8 mg/kg, at least about 8 mg/kg, at least about 9 mg/kg, or at least about 10 or more mg/kg.

In particular embodiments, the dose of ligand administered to a subject is at least about 0.001 μg/kg to at least about 10 mg/kg, at least about 0.01 μg/kg to at least about 10 mg/kg, at least about 0.1 μg/kg to at least about 10 mg/kg, at least about 1 μg/kg to at least about 10 mg/kg, at least about 0.01 mg/kg to at least about 10 mg/kg, at least about 0.1 mg/kg to at least about 10 mg/kg, or at least about 1 mg/kg to at least about 10 mg/kg, or any intervening range thereof.

In some aspects, a therapeutically effective amount of a ligand can be expressed as a molar concentration (i.e., M or mol/L). In some cases, a therapeutically effective amount of a ligand can be about 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 200 mM, 300 mM, 400 mM, 500 mM, 600 mM, 700 mM, 800 mM, 900 mM, 1000 mM or greater.

A therapeutically effective amount of a ligand can be administered once or more than once each day. In some cases, a therapeutically effective amount of a ligand is administered as needed (e.g., when pain relief is needed). The ligand may be administered serially (e.g., every day without a break for the duration of the treatment regimen). In some cases, the treatment regimen can be less than a week, a week, two weeks, three weeks, a month, or greater than a month. In some cases, a therapeutically effective amount of a ligand is administered for a day, at least two consecutive days, at least three consecutive days, at least four consecutive days, at least five consecutive days, at least six consecutive days, at least seven consecutive days, at least eight consecutive days, at least nine consecutive days, at least ten consecutive days, or at least greater than ten consecutive days. In a particular case, a therapeutically effective amount of a ligand is administered for three consecutive days. In some cases, a therapeutically effective amount of a ligand can be administered one time per week, two times per week, three times per week, four times per week, five times per week, six times per week, seven times per week, eight times per week, nine times per week, 10 times per week, 11 times per week, 12 times per week, 13 times per week, 14 times per week, 15 times per week, 16 times per week, 17 times per week, 18 times per week, 19 times per week, 20 times per week, 25 times per week, 30 times per week, 35 times per week, 40 times per week, or greater than 40 times per week. In some cases, a therapeutically effective amount of a ligand can be administered one time per day, two times per day, three times per day, four times per day, five times per day, six times per day, seven times per day, eight times per day, nine times per day, 10 times per day, or greater than 10 times per day. In some cases, a therapeutically effective amount of a ligand is administered at least every hour, at least every two hours, at least every three hours, at least every four hours, at least every five hours, at least every six hours, at least every seven hours, at least every eight hours, at least every nine hours, at least every 10 hours, at least every 11 hours, at least every 12 hours, at least every 13 hours, at least every 14 hours, at least every 15 hours, at least every 16 hours, at least every 17 hours, at least every 18 hours, at least every 19 hours, at least every 20 hours, at least every 21 hours, at least every 22 hours, at least every 23 hours, or at least every day. The dose of ligand may be administered to the subject continuously, or 1, 2, 3, 4, or 5 times a day; 1, 2, 3, 4, 5, 6, or 7 times a week, 1, 2, 3, or 4 times a month, once every 2, 3, 4, 5, or 6 months, or once a year, or at even longer intervals. The duration of treatment can last a day, 1, 2, or 3 weeks, 1, 2, 3, 4, 5, 7, 8, 9, 10, or 11 months, 1, 2, 3, 4, 5, or more years, or longer.

A subject treated by methods and compositions disclosed herein can be a human, or can be a non-human animal. The term “treat” and its grammatical equivalents used herein generally refer to the use of a composition or method to reduce, eliminate, or prevent symptoms of a disease and includes achieving a therapeutic benefit and/or a prophylactic benefit. By therapeutic benefit is meant slowing the progression of, halting the progression of, reversing the progression of, or eradication or amelioration of the symptoms of the disorder or condition being treated. A prophylactic benefit of treatment includes reducing the risk of a condition, retarding the progress of a condition, or decreasing the likelihood of occurrence of a condition.

Non-limiting examples of non-human animals include a non-human primate, a livestock animal, a domestic pet, and a laboratory animal. For example, a non-human animal can be an ape (e.g., a chimpanzee, a baboon, a gorilla, or an orangutan), an old world monkey (e.g., a rhesus monkey), a new world monkey, a dog, a cat, a bison, a camel, a cow, a deer, a pig, a donkey, a horse, a mule, a lama, a sheep, a goat, a buffalo, a reindeer, a yak, a mouse, a rat, a rabbit, or any other non-human animal. The compositions and methods as described herein are amenable to the treatment of a veterinary animal. A veterinary animal can include, without limitation, a dog, a cat, a horse, a cow, a sheep, a mouse, a rat, a guinea pig, a hamster, a rabbit, a snake, a turtle, and a lizard. In some aspects, contacting the tissue or cell population with a composition comprises administering the composition to a cell population or subject. In some embodiments, administration occurs in vitro, for example by adding the composition to a cell culture system. In some aspects, administration occurs in vivo, for example by administration through a particular route. Wherein more than one composition is to be administered, the compositions may be administered via the same route at the same time (e.g., on the same day), or via the same route at different times. Alternatively, the compositions may be administered via different routes at the same time (e.g., on the same day) or via different routes at different times.

The number of times a composition is administered to an subject in need thereof depends on the discretion of a medical professional, the disorder, the severity of the disorder, and the subject's response to the formulation. In some aspects, administration of a composition occurs at least once. In further aspects, administration occurs more than once, for example 2, 3, 4, 5, 6, 7, 8, 9, 10 or more times in a given period. The dosage of each administration and/or frequency of administrations may be adjusted as necessary based on the patient's condition and physiologically responses.

In some embodiments, compositions may be administered a sufficient amount of times to achieve a desired physiologic effect or improvement in a subject's condition. In the case wherein the subject's condition does not improve, upon the doctor's discretion the composition may be administered chronically, that is, for an extended period of time, including throughout the duration of the subject's life in order to ameliorate or otherwise control or limit the symptoms of the subject's disease or condition. In the case wherein the subject's status does improve, upon the doctor's discretion the composition may administered continuously; alternatively, the dose of drug being administered may be temporarily reduced or temporarily suspended for a certain length of time (i.e., a “drug holiday”). The length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. The dose reduction during a drug holiday may be from 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Where compositions are administered more than once, each administration may be performed by the same actor and/or in the same geographical location. Alternatively, each administration may be performed by a different actor and/or in a different geographical location.

With regard to human and veterinary treatment, the amount of a particular agent(s) that is administered may be dependent on a variety of factors, including the disorder being treated and the severity of the disorder; activity of the specific agent(s) employed; the age, body weight, general health, sex and diet of the patient; the time of administration, route of administration, and rate of excretion of the specific agent(s) employed; the duration of the treatment; drugs used in combination or coincidental with the specific agent(s) employed; the judgment of the prescribing physician or veterinarian; and like factors known in the medical and veterinary arts. Similarly, the effective concentration of a given composition may be dependent on a variety of factors including the age, sex, weight, genetic status, and overall health of the patient or subject.

Tables 2-8 below lists percent quench of YFP fluorescence following stimulation of the indicated engineered receptor by various doses of either acetylcholine or the non-native ligand.

TABLE 2

| Sequence of engineered | | | | |
|---|---|---|---|---|
| receptor | 50 nM abt | 100 nM abt | 300 nM abt | 1 uM abt |
| L131S_S172D in SEQ ID | −11.96 | −10.72 | 11.65 | −3.06 |
| NO: 33 | | | | |
| L131T_S172D in SEQ ID | −11.09 | −10.37 | −4 | 4.05 |
| NO: 33 | | | | |
| L131D_S172Din SEQ ID | −3.19 | 7.58 | −1.65 | 31.29 |
| NO: 33 | | | | |
| Y115D_S170Tin SEQ ID | −11.83 | −11.5 | −8.01 | −9.34 |
| NO: 33 | | | | |
| Y115D_L131Q in SEQ ID | −1.81 | −2.61 | −3.78 | −2.6 |
| NO: 33 | | | | |
| Y115D_L131E in SEQ ID | −1.68 | −2.25 | −2.37 | 3.68 |
| NO: 33 | | | | |

TABLE 3

| Sequence of engineered | | | | |
|---|---|---|---|---|
| receptor | 10 uM ach | 100 uM ach | 1 mM ach | 3 mM ach |
| L131S_S172D in SEQ ID | −11.3 | −27.53 | 2.84 | 14.65 |
| NO: 33 | | | | |
| L131T_S172D in SEQ ID | −12.24 | −26.23 | −1.16 | 2.97 |
| NO: 33 | | | | |
| L131D_S172D in SEQ ID | −12.91 | −20.88 | 79.85 | 79.98 |
| NO: 33 | | | | |
| Y115D_S170T in SEQ ID | −0.46 | −4.93 | −11.18 | −3.53 |
| NO: 33 | | | | |
| Y115D_L131Q in SEQ ID | 9.47 | 6.12 | −1.56 | 17.57 |
| NO: 33 | | | | |
| Y115D_L131E in SEQ ID | 15.45 | 13.77 | 27.42 | 53.62 |
| NO: 33 | | | | |

TABLE 4

| Sequence of engineered | | | |
|---|---|---|---|
| receptor | 100 nM apn | 1 uM apn | 30 uM apn |
| L131S_S172D in SEQ ID NO: | 14.26 | 45.55 | 64.93 |
| 33 | | | |
| L131T_S172D in SEQ ID NO: | 1.23 | 35.77 | 68.29 |
| 33 | | | |
| L131D_S172D in SEQ ID | 71.72 | 77.26 | 77.5 |
| NO: 33 | | | |
| Y115D_S170T in SEQ ID | −18.17 | −15.2 | −14 |
| NO: 33 | | | |
| Y115D_L131Q in SEQ ID | −10.03 | −3.33 | 6.96 |
| NO: 33 | | | |
| Y115D_L131E in SEQ ID | −5.71 | 13.03 | 25.2 |
| NO: 33 | | | |

TABLE 5

| Sequence of engineered | | | |
|---|---|---|---|
| receptor | 100 nM azd | 300 nM azd | 30 uM azd |
| L131S_S172D in SEQ ID | −9.32 | 4.94 | 60.61 |
| NO: 33 | | | |
| L131T_S172D in SEQ ID | −7.02 | −0.48 | 70.16 |
| NO: 33 | | | |
| L131D_S172Din SEQ ID | 51.44 | 77.12 | 79.26 |
| NO: 33 | | | |
| Y115D_S170T in SEQ ID | −0.71 | −1.73 | 47.39 |
| NO: 33 | | | |
| Y115D_L131Q in SEQ ID | 12.15 | 30.92 | 68.65 |
| NO: 33 | | | |
| Y115D_L131E in SEQ ID | 24.49 | 45.34 | 71.21 |
| NO: 33 | | | |

TABLE 6

| Sequence of | | | | | | | |
|---|---|---|---|---|---|---|---|
| engineered | 1 nM | 3 nM | 10 nM | 30 nM | 100 nM | 300 nM | 1 uM |
| receptor | brd | brd | brd | brd | brd | brd | brd |
| L131S_S172D in SEQ ID | −1.13 | −3.83 | −3.78 | | −2.03 | | 1.68 |
| NO: 33 | | | | | | | |
| L131D_S172D in SEQ ID | −1.24 | 0.25 | −2.57 | 0.78 | 4.51 | 30.97 | 44.41 |
| NO: 33 | | | | | | | |
| Y115D_S170T in SEQ ID | −9.78 | 12.06 | −9.02 | 16.3 | −7.44 | −10.93 | −4.55 |
| NO: 33 | | | | | | | |
| Y115D_L131Q in SEQ ID | 0.92 | −4.65 | 0.38 | 8.96 | 12.18 | −0.3 | 45.47 |
| NO: 33 | | | | | | | |
| Y115D_L131E in SEQ ID | −1.52 | −5.35 | −3.49 | 2.16 | 4.18 | 70.2 | 43.11 |
| NO: 33 | | | | | | | |

TABLE 7

| Sequence of engineered | 30 nM | 100 nM | 300 nM | 10 uM | 30 uM |
|---|---|---|---|---|---|
| receptor | fac | fac | fac | fac | fac |
| L131S_S172D in SEQ ID | −23.44 | | 2.75 | 69.71 | 61.44 |
| NO: 33 | | | | | |
| L131T_S172D in SEQ ID | −21.36 | | 4.17 | | 65.94 |
| NO: 33 | | | | | |
| L131D_S172D in SEQ ID | 24.15 | | 80.17 | 81.34 | 79.9 |
| NO: 33 | | | | | |
| Y115D_S170T in SEQ ID | 38.08 | −14.37 | 19.41 | 67.33 | 79.67 |
| NO: 33 | | | | | |
| Y115D_L131Q in SEQ ID | 41.32 | 59.51 | 74.86 | 78.53 | 84.45 |
| NO: 33 | | | | | |
| Y115D_L131E in SEQ ID | 45.76 | 63.92 | 79.43 | 80.1 | 84.91 |
| NO: 33 | | | | | |

TABLE 8

| Sequence of engineered | | | |
|---|---|---|---|
| receptor | 1 uM tc6 | 10 uM tc6 | 500 uM tc6 |
| L131S_S172D in SEQ ID NO: | −20.99 | −27.62 | −24.22 |
| 33 | | | |
| L131T_S172D in SEQ ID NO: | −21.8 | −27.08 | −21.56 |
| 33 | | | |
| L131D_S172D in SEQ ID NO: | −18.24 | −23.06 | −19.3 |
| 33 | | | |
| Y115D_S170T in SEQ ID NO: | 31.9 | 25.54 | 21.13 |
| 33 | | | |
| Y115D_L131Q in SEQ ID NO: | 38.83 | 31.59 | 29.83 |
| 33 | | | |
| Y115D_L131E in SEQ ID NO: | 36.07 | 31.35 | 28.18 |
| 33 | | | |

Tables 9-11 below list the EC50 of the indicated engineered receptor calculated by different techniques as indicated.

TABLE 9
EC50 values measured from YFP quench plate reader experiment

| SEQ ID | Sequence of the engineered | EC50 to | | EC50 to | |
|---|---|---|---|---|---|
| NO: | receptor | ACh | | TC-5619 | |
| 58 | L131D, S172D in SEQ ID NO: 33 | 359 | uM | 757 | nM |
| 59 | L131S, S172D in SEQ ID NO: 33 | >3 | mM | >1 | uM |
| 60 | L131T, S172D in SEQ ID NO: 33 | >3 | mM | NA | |
| 61 | Y115D, L131E in SEQ ID NO: 33 | 1.74 | mM | 149 | nM |
| 62 | Y115D, L131Q in SEQ ID NO: 33 | 4.61 | mM | 968 | nM |
| 63 | Y115D, S170T in SEQ ID NO: 33 | >3 | mM | >l | uM |

TABLE 10
EC50 values measured from high throughput electrophysiology experiment

| | EC50 (uM) | | | |
|---|---|---|---|---|
| Description of sequence | Acetylcholine | AZD-0328 | RG-3487 | TC-6987 |
| Wild type nAchRa7 | 47.9 | 1.2 | 2.20 | 3.50 |
| L131Sin | 1759 | 0.45 | 0.73 | 26.5 |
| SEQ ID NO: 33 | | | | |
| L13 IT in | 6015 | 2.92 | 1.76 | no |
| SEQ ID NO: 33 | | | | activity |
| L131D in | 532 | 0.33 | 0.41 | 5.42 |
| SEQ ID NO: 33 | | | | |
| S172D in | 1037 | 0.30 | 0.39 | no |
| SEQ ID NO: 33 | | | | activity |
| L131S, S172D in | 14260 | 3.89 | 0.95 | no |
| SEQ ID NO: 33 | | | | activity |
| L131T, S172D in | 22014 | 5.50 | 2.65 | no |
| SEQ ID NO: 33 | | | | activity |
| L131D, S172D in | 306 | 0.15 | 0.07 | no |
| SEQ ID NO: 33 | | | | activity |
| Y115D in | 26887 | no | 0.70 | no |
| SEQ ID NO: 33 | | activity | | activity |
| Y115E in | 2295 | no | 0.10 | no |
| SEQ ID NO: 33 | | activity | | activity |
| Y115D, S170T in | 30000 | no | 2.47 | no |
| SEQ ID NO: 33 | | activity | | activity |
| Y115D, L131Q in | 28386 | no | 0.17 | no |
| SEQ ID NO: 33 | | activity | | activity |
| Y115D, L131E in | 4636 | no | 0.13 | no |
| SEQ ID NO: 33 | | activity | | activity |

TABLE 11

| | | | Manual Patch Clamp | | Rheobase Shift; Rat | |
|---|---|---|---|---|---|---|
| Sequence | Manual Patch Clamp EC50 | | EC50 | | DRG Neurons | |
| of the | HEK Cell Line | | Rat DRG Neurons | | TC5619 @ EC90 | |
| engineered | Facinicline/ | | Facinicline/ | | | +TC- |
| receptor | RG3487 | Acetylcholine | RG3487 | Acetylcholine | Baseline | 5619 |
| Y115D, | 0.29 μM | >30,000 μM | 0.36 μM | >100,000 μM | 163 ± 28 | 625 ± 35 |
| L131Q | | | | | pA | pA |

| | | |
|---|---|---|
| Y115D, | 0.23 μM | 2200 μM |
| L131E | | |

Table A below lists the double mutants disclosed herein.

TABLE A

| SEQ ID NO: | Sequence |
|---|---|
| SEQ ID NO: 58 | L131D, S172D in SEQ ID NO: 33 |
| SEQ ID NO: 59 | L131S, S172D in SEQ ID NO: 33 |
| SEQ ID NO: 60 | L131T, S172D in SEQ ID NO: 33 |
| SEQ ID NO: 61 | Y115D, L131E in SEQ ID NO: 33 |
| SEQ ID NO: 62 | Y115D, L131Q in SEQ ID NO: 33 |
| SEQ ID NO: 63 | Y115D, S170T in SEQ ID NO: 33 |

All papers, publications and patents cited in this specification are herein incorporated by reference as if each individual paper, publication or patent were specifically and individually indicated to be incorporated by reference and are incorporated herein by reference to disclose and describe the methods and/or materials in connection with which the publications are cited. However, mention of any reference, article, publication, patent, patent publication, and patent application cited herein is not, and should not be taken as an acknowledgment or any form of suggestion that they constitute valid prior art or form part of the common general knowledge in any country in the world.

Unless the context indicates otherwise, it is specifically intended that the various features described herein can be used in any combination.

Unless otherwise defined, all technical and scientific terms used herein have the same meaning as commonly understood by one of ordinary skill in the art to which this disclosure belongs.

It is to be understood that the description above as well as the examples that follow are intended to illustrate, and not limit, the scope of the invention. Other aspects, advantages and modifications within the scope of the invention will be apparent to those skilled in the art to which the invention pertains.

### EXAMPLES

### Example 1. Discovery of Engineered Receptors Comprising Mutations in the Ligand Binding Domain

To generate LGICs that conduct anion current following exposure to non-native small molecule agonists of the human α7-nAChR, chimeric ligand gated ion channel (LGIC) receptors comprising the ligand binding domain derived from the human α7 nicotinic acetylcholine receptor (α7-nAChR) and the chloride-conducting ion pore domain derived from the human GlyR1α were genetically engineered. An engineered receptor with an amino acid sequence of SEQ ID NO: 33 was identified, which was approximately as sensitive to acetylcholine, ABT-126 and TC-6987 as wild type α7-nAChR, with TC-6987 showing partial agonist activity on SEQ ID NO:33 similar to wild type. SEQ ID NO:33 was approximate 2-fold less sensitive to nicotine and approximately 3-fold and 10-fold more sensitive to AZD-0328 and Facinicline/RG3487, respectively, than wild type.

Amino acid substitutions were introduced into the ligand binding domain of the engineered receptor with the amino acid sequence of SEQ ID NO: 33. The binding pocket of each ligand in the α7-nAChR was modeled, and the amino acid residues that form the binding pocket were mapped. Libraries of single, double and triple mutant chimeric LGICs were then generated, each mutant chimeric LGIC comprising substitutions in one or more amino acids of the ligand binding pocket of SEQ ID NO: 33. The parental chimera receptor (SEQ ID NO: 33) was cloned into pcDNA3.1(+) (Invitrogen) using BamHI and EcoRI sites by standard molecular biology techniques. Amino acid substitutions were introduced by site-directed mutagenesis.

All the resulting engineered receptors were analyzed for their potency to their native ligand, acetylcholine (Ach), and non-native ligands such as, AZD-0328 (adisinsight. springer. com/drugs/800018503), TC-6987 (drugbank.ca/drugs/DB 14854), ABT-126 (medchemexpress.com/Nelonicline.html), APN-1125 (clinicaltrials.gov/ct2/show/NCT02724917), TC-5619 (en.wikipedia.org/wiki/Bradanicline), and Facinicline/RG3487 (researchgate.net/figure/Molecular-structure-of-RG3487_fig1_47499934).

### Example 2: Characterization of the Engineered Receptors Using High Throughput Fluorescence Based Plate Screening

To screen these mutant LGICs for those having novel response profiles to ligands, an anion reporter assay was developed to assess the function of channels in a high throughput format. In this assay, cells expressing a YFP reporter whose fluorescence is quenched in the presence of anion are transfected with DNA encoding the channel of interest. Upon exposure to ligand, channels that are activated will flux anion, resulting in a dose-dependent quench of the YFP that can be detected on a plate reader.

Lenti-X 293T cells (LX293T, Clontech) were maintained in DMEM containing 10% FBS and 1% penicillin/streptomycin (Invitrogen). For plate reader assays, LX293T cells were infected with a lentivirus to create cells that stably express a mutant YFP (H148Q/I152L) reporter, which displays enhanced sensitivity to anions. Two days before assay, cells were split at a density of 20,000 cells/well in a 96-well tissue culture plate coated with poly-d lysine (Thermo Scientific). The next day, the cells were transiently transfected with 0.1 μg of DNA per well using standard Fugene protocol (Promega). On the day of assay, cells were washed 2 times in 1× extracellular solution (1×ECS: 140 mM NaCl, 5 mM KCl, 1 mM MgCl2, 2 mM CaCl2, 10 mM HEPES, 10 mM glucose, pH 7.2, mOsms 300). After the last wash, 100 μL of 1×ECS was added to the wells and the plate was incubated at 37° C. for 30 min. While incubating the plate, the drugs were diluted to a 2× concentration in 1×ECS-NaI (same components as 1×ECS except the 140 mM NaCl was replaced by 140 mM NaI). The plates were then read on a Flexstation3 (Molecular Devices). Each well, 8 wells at a time, of the plate is read for 2 min using a Flexstation3 (Molecular Devices) as follows: 1) a baseline YFP fluorescence is read for 17 sec, 2) 100 μL of ligand is added, and 3) the changes in YFP fluorescence are then measured every 1.3 sec for the remaining time.

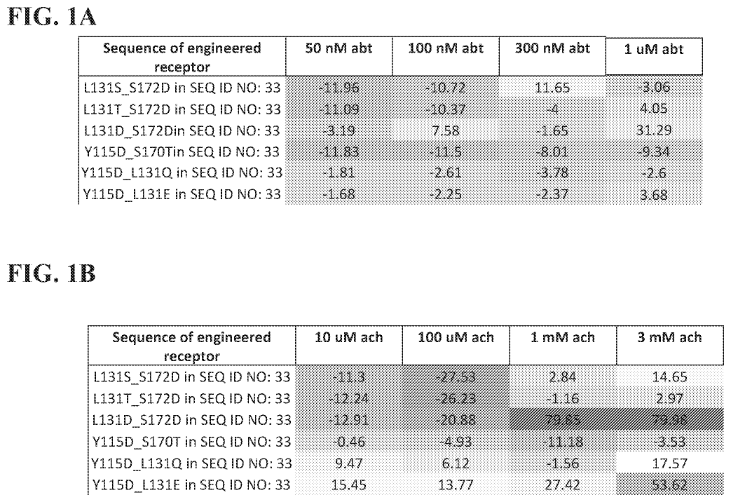

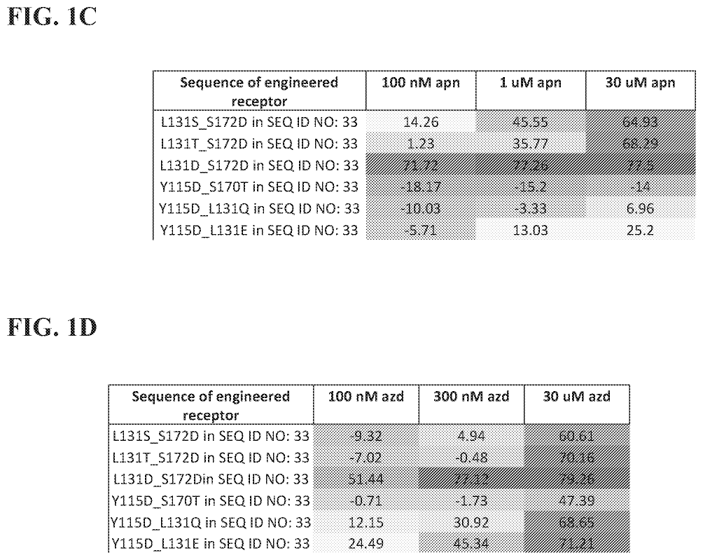

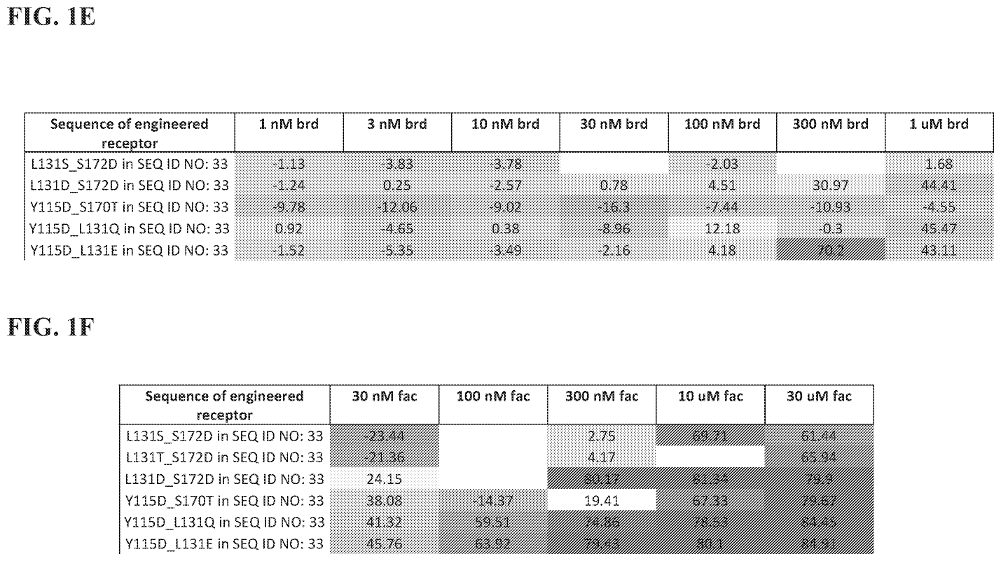

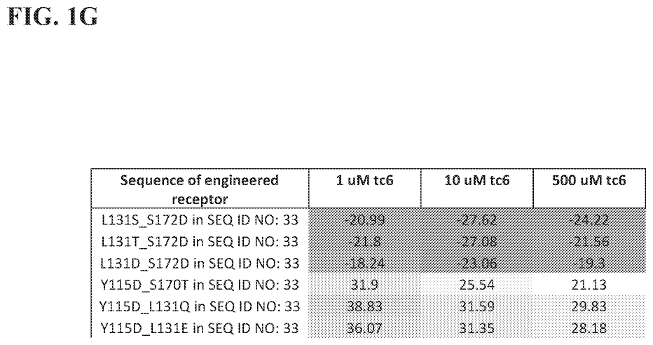

FIG. 1 provides heat maps of the percent quench of YFP fluorescence following stimulation by various doses of either acetylcholine or the non-native ligand as indicated in the Figure. A positive fluorescence signal, as indicated by blue shaded cells, indicate that the engineered receptor is activated by the non-native ligand at that concentration. The results indicate that the engineered receptors have varying potency towards the non-native ligands tested. Table 12 lists the EC50 values for the indicated double mutants to acetylcholine (Ach) and TC-5619 as determined from the YFP fluorescence plate reader experiment.

TABLE 12

| | EC50 to | | EC50 to | |
|---|---|---|---|---|
| Sequence of the engineered receptor | ACh | | TC-5619 | |
| L131D, S172Din SEQ ID NO: 33 | 359 | uM | 757 | nM |
| L131S, S172D in SEQ ID NO: 33 | > 3 | mM | >1 | uM |
| L131T, S172D in SEQ ID NO: 33 | >3 | mM | NA | |
| Y115D, L131E in SEQ ID NO: 33 | 1.74 | uM | 149 | nM |
| Y115D, L131Q in SEQ ID NO: 33 | 4.61 | uM | 968 | nM |
| Y115D, S170T in SEQ ID NO: 33 | >3 | mM | >1 | uM |

### Example 3: Characterization of the Engineered Receptors Using High Throughput Electrophysiology

To confirm the EC₅₀s determined by plate reader as well as develop a better understanding of maximum current flow, the engineered receptors were subjected to high throughput electrophysiology system, as described below. For HEK293T studies, cDNAs encoding ion channels were cloned into pcDNA3.1 using standard recombination techniques. HEK293T cells from Clontech (Lenti-X™ 293T Cell Line) were cultured in DMEM supplemented with 10% FBS and 1% Pen/Strep to 40-50% confluence using standard cell culture protocols, transfected with ion channel plasmids at a concentration of 18 μg per 15 cm dish using Fugene 6, and grown for an additional 24 hours. Cells were then assayed on a electrophysiological system (IonFluxHT and/or Mercury, Fluxion Biosciences) in which dose-response relationships may be assessed through a microfluidics-based platform for establishing whole cell configurations. Ensemble plates were primed with extracellular buffer (140 mM NaCl, 5 mM KCl, 2 mM CaCl₂), 1 mM MgCl₂, 10 mM HEPES, and 10 mM glucose, pH 7.2 with NaOH, mOsm 310), intracellular buffer (145 mM CsCl, 2 mM CaCl2, 2 mM MgCl2, 10 mM HEPES, and 10 mM EGTA, pH 7.2 with CsOH, mOsm 305), as adapted from Lynagh and Lynch, and test compounds (stocks prepared fresh) diluted in extracellular buffer. Cells were then released from the plate with Accutase, centrifuged, resuspended in extracellular buffer, and loaded into Ensemble plates. Cells were then subjected to a standard protocol for priming, trapping, breaking, and establishing whole cell configuration, with cells held at −60 mV throughout the recording. After recording baseline, progressive doses of test compounds were applied using the IonFlux software to assess dose response relationships. Data were then analyzed off-line using custom Python scripts to convert data to .csv format, re-plot traces, and apply QC measurements to reject unstable recordings (i.e. thresholding based on access resistance and/or standard deviation in baseline, as well as artifact rejection). Peak currents were then calculated and population data were fit using a 4-parameter logistic equation as described by the Hill Equation. Currents were measured on an automated patch clamp system (Fluxion Biosciences) following 1 second addition of drug and the EC50 values calculated are tabulated below in Table 13.

TABLE 13

| | EC50 (uM) | | | |
|---|---|---|---|---|
| Description of sequence | Acetylcholine | AZD-0328 | RG-3487 | TC-6987 |
| Wild type nAchRa7 | 47.9 | 1.2 | 2.20 | 3.50 |
| L131S in | 1759 | 0.45 | 0.73 | 26.5 |
| SEQ ID NO: 33 | | | | |
| L13 IT in | 6015 | 2.92 | 1.76 | no |
| SEQ ID NO: 33 | | | | activity |
| L131D in | 532 | 0.33 | 0.41 | 5.42 |
| SEQ ID NO: 33 | | | | |
| S172D in | 1037 | 0.30 | 0.39 | no |
| SEQ ID NO: 33 | | | | activity |
| L131S, S172D in | 14260 | 3.89 | 0.95 | no |
| SEQ ID NO: 33 | | | | activity |
| L131T, S172D in | 22014 | 5.50 | 2.65 | no |
| SEQ ID NO: 33 | | | | activity |
| L131D, S172D in | 306 | 0.15 | 0.07 | no |
| SEQ ID NO: 33 | | | | activity |
| Y115D in | 26887 | no activity | 0.70 | no |
| SEQ ID NO: 33 | | | | activity |
| Y115E in | 2295 | no activity | 0.10 | no |
| SEQ ID NO: 33 | | | | activity |
| Y115D, S170T in | 30000 | no activity | 2.47 | no |
| SEQ ID NO: 33 | | | | activity |
| Y115D, L131Q in | 28386 | no activity | 0.17 | no |
| SEQ ID NO: 33 | | | | activity |
| Y115D, L131E in | 4636 | no activity | 0.13 | no |
| SEQ ID NO: 33 | | | | activity |

These results show that all the engineered receptors have reduced potency to acetylcholine as compared to wild type nAchRa7. For instance, some of the engineered receptors have EC50 values that are several orders of magnitudes higher than the EC50 of wild type nAchRa7. Furthermore, the results show that some of the engineered receptors have increased potency to certain non-native ligands, as compared to the wild type receptor. For instance, the engineered receptor comprising the amino acid sequence of L131D, S172D in SEQ ID NO: 33 shows at least 10-fold increased potency towards AZD-0328 and RG-3487, as compared to the wild type control receptor. These results demonstrate that the engineered receptors may be used to decrease potency to acetylcholine, while simultaneously retaining or increasing the potency to synthetic small molecule nACha7 receptor agonists, which have been recognized as being safe and well tolerated in humans.

These results from electrophysiological methods provide confirmation of the EC50 values from the plate reader, and further confirm the decoupling of acetylcholine and non-native ligand responses of the engineered receptors disclosed herein.

### Example 4: Characterization of the Engineered Receptors Using Manual Patch Clamp Electrophysiology

Using whole cell manual patch clamp electrophysiology in HEK293 cells and rat DRG neurons, the EC50 of the various engineered receptors disclosed herein to Ach and non-native ligands was measured. The rheobase shift was also measured in rat DRG neurons, which reflects the efficacy of the receptors. To calculate the rheobase, first, the amount of current that can produce an action potential was determined. Then the ligand was added, followed by a step wise increase in the current injected up to 700 pA. The current injected after addition of the ligand was divided by, the original current injected to obtain an action potential, to calculate the rheobase. The EC50 was calculated using the Hill equation with peak current measurements from escalating doses of ligand.

The results are tabulated below in Table 14. These results confirm that the engineered receptors disclosed herein have very low potency to Ach, but higher potency and efficacy to non-native ligands such as RG3487 and TC5619. See also, Example 6.

TABLE 14

| | | | Manual Patch Clamp | | Rheobase Shift; Rat | |
|---|---|---|---|---|---|---|
| Sequence | Manual Patch Clamp EC50 | | EC50 | | DRG Neurons | |
| of the | HEK Cell Line | | Rat DRG Neurons | | TC5619 @ EC90 | |
| engineered | Facinicline/ | | Facinicline/ | | | +TC- |
| receptor | RG3487 | Acetylcholine | RG3487 | Acetylcholine | Baseline | 5619 |
| Y115D, | 0.29 μM | >30,000 μM | 0.36 μM | >100,000 μM | 163 ± 28 | 625 ± 35 |
| L131Q | | | | | pA | pA |

| | | |
|---|---|---|
| Y115D, | 0.23 μM | 2200 μM |
| L131E | | |

### Example 5: Localization of the Engineered Receptors

The efficiency with which the engineered receptors disclosed herein are localized to the surface of the cells was evaluated using two methods. First, HA-tagged engineered receptors were expressed in HEK293T cells and their surface expression was monitored using fluorescently tagged HA antibodies. Second, fluorescently labelled α-bungarotoxin, which specifically binds to amino acids on the engineered receptors (CHNRA7) was used to bind to the engineered receptors. The use of both these methods, followed by flow cytometry, allowed corroboration of the surface localization of the engineered receptors.

Monoclonal antibodies anti-HA-PeCy7 (16B12) were purchased from Biolegend (San Diego, Calif.). The monoclonal antibody clone name is listed in parentheses. alpha-bungarotoxin conjugated to biotin, Alexa Fluor 647 were all purchased from Thermo/Fisher (Waltham Mass.). Briefly, HEK-293T were plated at 200,00 cells per well and transfected with Fugene 6 the next day at a 1:3 ratio of DNA to Fugene. Cells were analyzed the day after transfection using flow cytometry. For flow cytometry analysis, transfected HEK293T cells were lifted using 0.05% Trypsin and 0.02% EDTA (Thermo/Fisher), washed and incubated in antibody (30 min, 1:100) or α-bungarotoxin at (1 hour. 1:1000) in FACS Buffer (2% BSA, 1×PBS without Ca+ and Mg+, and 1× Penicillin Streptomycin). Cells were then washed in FACS buffer and then analyzed on a Sony SH800 FACS sorter (San Jose, Calif.). Subsequent analysis was done using FlowJo (San Jose, Calif.). Data presented are normalized to the % of cells positive for HA-tag fluorescence or α-bungarotoxin staining; and the median fluorescent intensity of the parental chimeric receptor comprising the amino acid sequence of SEQ ID NO: 33. See Table 1.

FIG. 5 and Table 1 show % of HA-tag positive cells that are expressing the engineered receptors, normalized to control cells expressing the amino acid sequence of SEQ ID NO: 33 (“Average HA tag %”); and the % of α-bungarotoxin positive cells that are expressing the engineered receptors, normalized to control cells expressing the amino acid sequence of SEQ ID NO: 33 (“Normalized AB %”).

FIG. 5 and Table 15 also show the median fluorescent intensity (MFI) of a cell expressing the engineered receptors, normalized to control cells expressing the amino acid sequence of SEQ ID NO: 33, as evaluated using anti-HA antibodies (“Average HA MFI”) or fluorescently labeled α-bungarotoxin conjugated to Alexa Fluor 647 (“Normalized AB MFI”). The different point mutations can influence the detection of both HA and alpha-bungarotoxin by flow cytometry.

TABLE 15

| Sequence of the | Average | Average HA | | Normalized | Normalized | |
|---|---|---|---|---|---|---|
| Engineered Receptor | HA tag % | MFI | N | AB % | AB MFI | N |
| L131S, S172D in | 33 | 0.77 | 2 | 48 | 0.83 | 4 |
| SEQ ID NO: 33 | | | | | | |
| L131T, S172D in | 92 | 0.93 | 1 | 32 | 0.75 | 4 |
| SEQ ID NO: 33 | | | | | | |
| L131D, S172D in | 58 | 0.82 | 1 | 45 | 0.88 | 4 |
| SEQ ID NO: 33 | | | | | | |
| Y115D, S170T in | 0 | 0.80 | 1 | 17 | 0.64 | 1 |
| SEQ ID NO: 33 | | | | | | |
| Y115D, L131Q in | 62 | 0.76 | 2 | 35 | 0.72 | 3 |
| SEQ ID NO: 33 | | | | | | |
| Y115D, L131E in | 87 | 0.92 | 1 | 73 | 0.90 | 1 |
| SEQ ID NO: 33 | | | | | | |

*Abbreviations: MFI-Mean Fluorescence Intensity; AB-α-bungarotoxin

The results show that the mutations in the engineered receptors disclosed herein affect the localization to the surface of the cell. While the localization of some double mutants is comparable to that of the parental chimera (SEQ ID NO: 33), others have diminished cell surface localization compared to the parental chimera (SEQ ID NO: 33). For instance, the engineered receptor with the L131T, S172D mutations shows comparable localization to the parental chimera as evaluated by the HA tag. Moreover, the engineered receptor with the Y115D, L131E mutations shows comparable localization to the parental chimera by both techniques, that is, as evaluated by the HA tag as well as the α-bungarotoxin.

### Example 6: Characterization of the CR-11 Engineered Receptor

The high throughput electrophysiology platform indicated that the potency of the engineered receptor comprising an amino acid sequence with amino acid substitutions of Y115D and L131Q in SEQ ID NO: 33 to acetylcholine is over 500 fold reduced as compared to the wild type receptor, while its potency to RG-3487 is increased by over 10-fold.

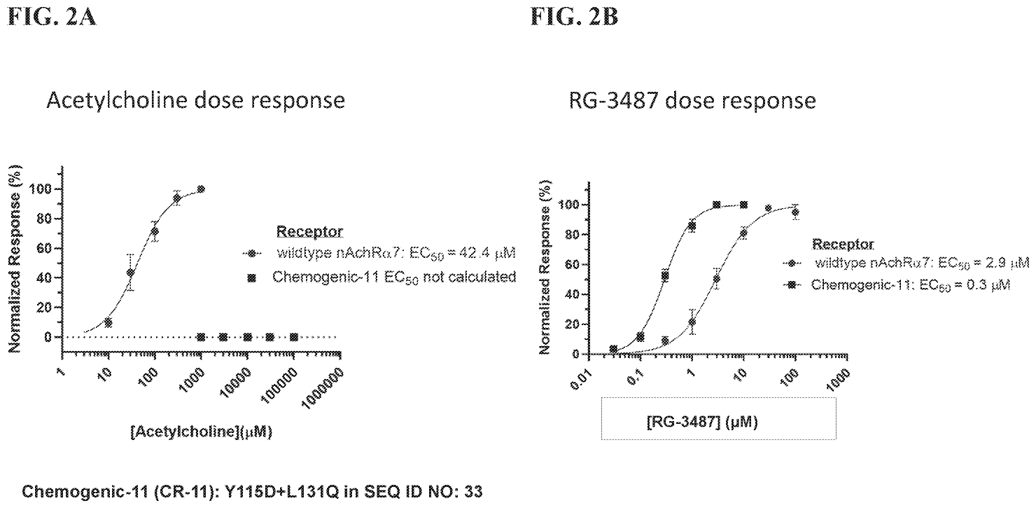

Using whole cell manual patch clamp electrophysiology, in both HEK293 cells and cultured rat dorsal root ganglion (DRG) sensory neurons, it was confirmed the engineered receptor comprising an amino acid sequence with amino acid substitutions of Y115D and L131Q in SEQ ID NO: 33 is essentially insensitive to acetylcholine, but way more sensitive to RG-3487, as compared to the wildtype nAchRa7 receptor. See FIG. 2.

In HEK293 cells, the EC₅₀ of the engineered receptor comprising an amino acid sequence with amino acid substitutions of Y115D and L131Q in SEQ ID NO: 33 to ACh could not be determined because almost no current could be generated, even at concentration of Ach up to 100 mM. In contrast, the EC₅₀ of the wildtype nAchRa7 receptor to ACh is 42.4 μM (FIG. 2A). Further confirming the high throughput data, the manual patch clamp electrophysiology results also shows that the engineered receptor comprising an amino acid sequence with amino acid substitutions of Y115D and L131Q in SEQ ID NO: 33 is more sensitive to RG-3487 (SA-2) (with an EC₅₀=0.3 μM), as compared to the wildtype nAchα7 receptor (EC₅₀=2.9 μM) (FIG. 2B).

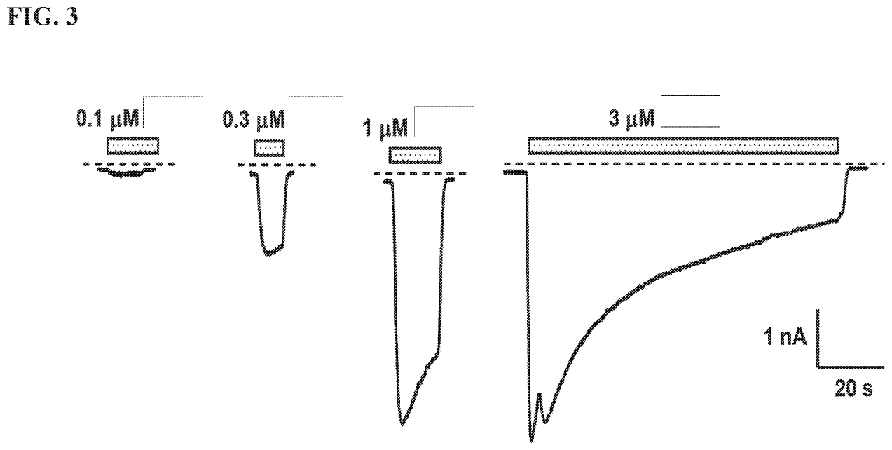

In cultured adult rat DRG neurons expressing the engineered receptor comprising an amino acid sequence with amino acid substitutions of Y115D and L131Q in SEQ ID NO: 33, application of RG-3487 (SA-2) generated chloride current in a dose dependent manner (FIG. 3), although such a current is not observed in non-transduced cells.

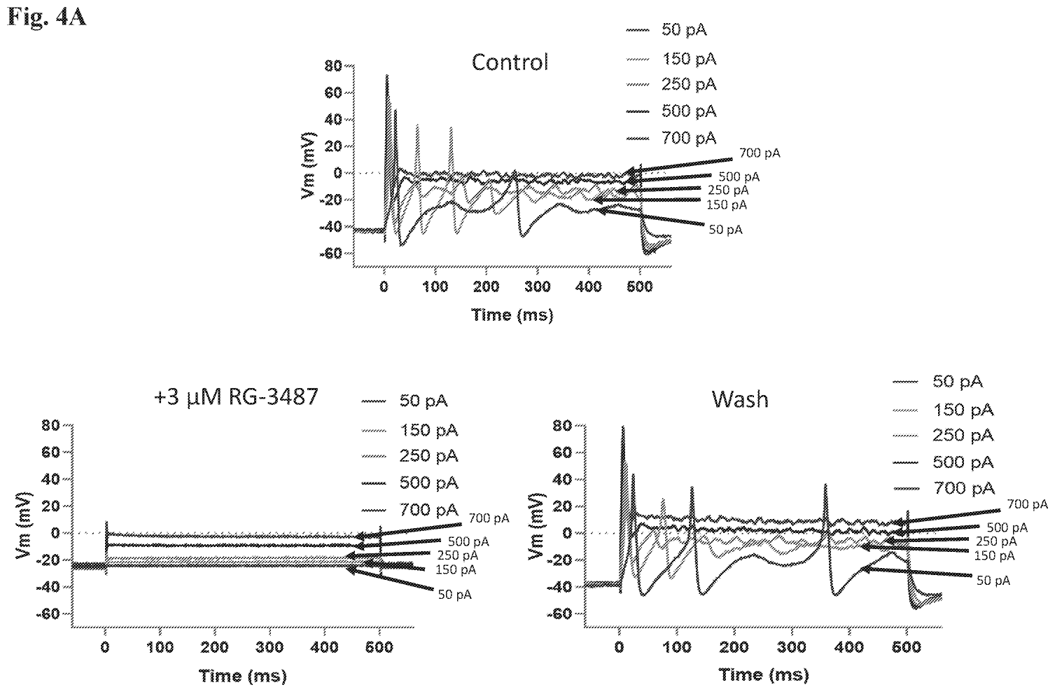

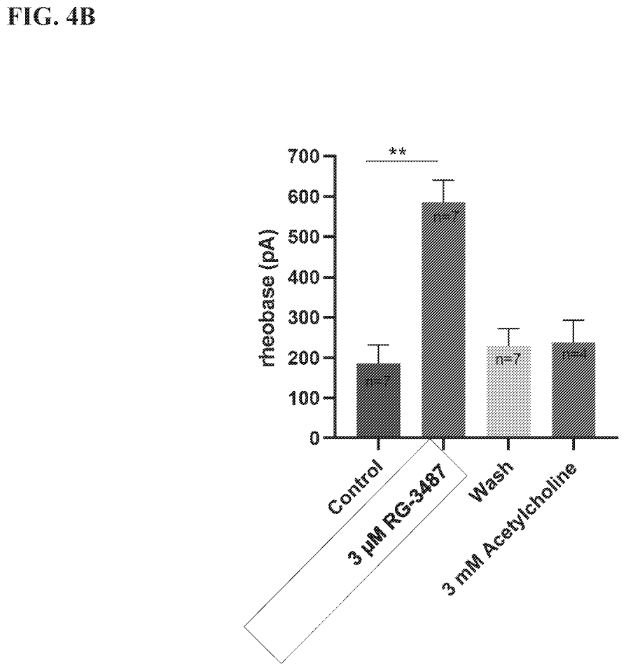

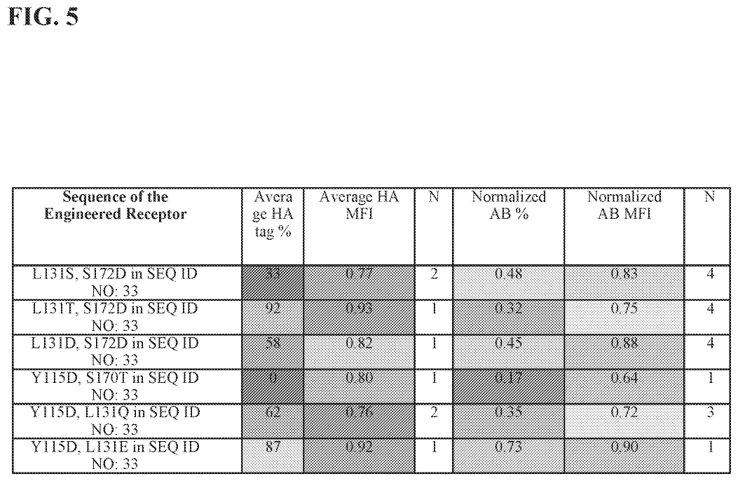

Additionally, activation of the engineered receptor comprising an amino acid sequence with amino acid substitutions of Y115D and L131Q in SEQ ID NO: 33 with RG-3487 (SA-2) at 3 μM concentration in transduced rat DRG neurons reversibly inhibits current injection evoked action potentials (FIG. 4A) with an approximate 3× increase in the current required to elicit action potentials (n=7). Acetylcholine at 3.0 mM had no effect on evoked action potentials in transduced neurons (n=4) (FIG. 4B). These results show that expression of the engineered receptor comprising an amino acid sequence with amino acid substitutions of Y115D and L131Q in SEQ ID NO: 33 in DRG neurons can inhibit evoked action potential.

### Example 7. Characterization of Engineered Receptors in IPSC-Derived Neurons (Prophetic)

Differentiation protocols to generate IPSC-derived AP neurons are developed. The following markers are used to define the cells as AP neurons; expression of Neurofilament 200 (NF200), which delineates myelinated primary afferent neurons (Basbaum et al, 2009), Piezo 2, a marker of Low Threshold Mechanoreceptor sensory neurons (LTMRs) (Ranade et al., 2014) and TLR5, a toll-like receptor that reportedly also marks Aβ fibers (Xu et al., 2015). The characterization will also include evaluation for absence of the nociceptor specific marker TrpV1, which is expressed in many C and Aβ fibers (Caterina et al., 1997), prostatic acid phosphatase, which delineates non-peptidergic unmyelinated afferents (Zylka et al., 2009), and NaV1.1, a marker of Aβ nociceptive neurons. IPSC-derived neurons that meet the above criteria will be further characterized as either rapidly adapting LTMRs, based on expression of c-Ret and MafA/C-Maf, or slowly adapting LTMRs, based on expression of TrkB and Shox2, in the absence of c-Ret expression (Koch et al., 2018).

(1) Confirm that cells meeting the above expression marker criteria of Aβ neurons have electrophysiology properties characteristic of non-nociceptive sensory neurons. These properties include generation of current in response to ligand and that direct current injection evokes action potentials.

(2) Confirm presence of chloride currents in neurons transduced with select chemogenetic receptors (engineered receptors). Cells are transduced with a lentivirus vector encoding an HA-tagged chemogenetic receptor(s) with an IRES GFP to identify transduced cells via GFP fluorescence. Chloride currents in response to Synthetic Agonists are detected in voltage clamp mode with NMGD+ as the internal/external fluid cation. As NMDG+ is impermeable to cation channels, its inclusion eliminates any endogenous nAchRa7 cation currents in response to the Synthetic Agonists studied. The EC50 for Chemogenetic Receptor-Synthetic Agonist pairs is then determined. Following recordings, expression and cell surface localization of the receptor(s) are evaluated by fluorescence microscopy in permeabilized and non-permeabilized cells using antibodies directed at the HA tag.

(3) Assess ability to inhibit current injection evoked action potentials. Cells are transduced with a lentivirus vector encoding an HA-tagged chemogenetic receptor(s) with an IRES GFP to identify transduced cells via GFP fluorescence. In current clamp mode, by escalating application of current until the cell membranes depolarizes, the rheobase in the presence and absence of the synthetic agonist is determined. Results are compared to cells that are transduced with GFP only.

(4) Assess impact on input resistance. Cells are transduced with a lentivirus vector encoding an HA-tagged chemogenetic receptor(s) with an IRES GFP to identify cells via GFP fluorescence. In current clamp mode, to calculate the input resistance, sub threshold current is injected and resulting change in membrane voltage is determined. Results are compared to cells that are transduced with GFP only.

(5) Assess impact on resting membrane potential in the presence and absence of synthetic agonist. Cells are transduced with a lentivirus vectors encoding an HA-tagged chemogenetic receptor(s) with an IRES GFP to identify transduced cells via GFP fluorescence. In voltage clamp mode the resting membrane potential is determined in the presence and absence of the synthetic agonist. Results are compared to cells that are transduced with GFP only.

The above electrophysiology properties in the IPSC-derived Aβ neurons are compared with those in IPSC-derived C-fiber neurons as well as adult rat DRG neurons. In addition, as biochemical changes in the injured Aβ fiber afferents can contribute to spontaneous pain in neuropathic states, the electrophysiological properties of the IPSC derived Aβ neurons is investigated following injury in vitro. To produce the injuries in vitro, cells are harvested and re-plated after processes have been extended in culture. The re-plating process severs the processes, mimicking an axonal injury. At various time point post injury the cells are evaluated for various electrophysiology properties including: generation of spontaneous action potentials, changes in resting membrane potentials and changes in the rheobase. The effect of the chemogenetic receptors are evaluated in the injured state as well.

### Example 8. Assessing the Efficacy of Engineered Receptors to Treat Disease in Animal Models

The engineered receptors disclosed herein are assessed for their ability to provide analgesia in a rat model of neuropathic pain following administration of a small molecule ligand. AAV expression cassettes containing a human synapsin-1 (hSYN) promoter linked to polynucleotides encoding either a wild type α7-nAChR, or any one of the engineered chimeric receptors disclosed herein are constructed using standard molecular biology techniques.

These AAV expression cassettes are subcloned into AAV bacmids, purified, transfected into Sf9 insect cells to produce recombinant baculovirus, and then amplified. Sf9 cells are double infected with the amplified recombinant baculovirus containing with either the wild type α7-nAChR, or any one of the engineered chimeric receptors cassettes described above, and another recombinant baculovirus containing the Rep and AAV6 (Y705+731F+T492V) Cap genes to produce recombinant AAV vectors. The viral vectors are purified, viral titer determined using qPCR, and SDS-PAGE used to verify the purity of AAV vectors.

Behavioral experiments and pain models: To produce mechanical hypersensitivity in a model that mimics a neuropathic pain condition, the spared nerve injury (SNI) model (a validated model of mechanical allodynia) is used (Shields et al., 2003, The Journal of Pain, 4, 465-470). This model is produced by the sectioning of the common peroneal and the sural nerves and isolating the tibial branch. Mechanical withdrawal threshold is assessed by placing rats on an elevated wire-mesh grid and stimulating the plantar surface of the hind paw with von Frey filaments.

AAV injection into the spinal cord of rats: A dorsal hemilaminectomy is made at the level of the lumbar enlargement to expose two segments (about 1.5-2 mm) of lumbar spinal cord, after which the dura mater is incised and reflected. The viral solution is loaded into a glass micropipette (prefilled with mineral oil). The micropipette is connected to a manual micro-injector mounted on a stereotactic apparatus. The viral solution is targeted to the dorsal horn (left side). Along the rostro-caudal axis within the exposed region, 6 injections of 240 nl each are performed, in an equidistant linear fashion. After each injection, 1 min of resting time is observed and then the muscle layer is sutured, the skin closed with staples, and the animals were allowed to recover with heated-pad before they were returned to their home cages. Animals are perfused for histological analysis after the last behavior test.

AAV intraganglionic injections into the dorsal root ganglion (DRG) of rats: The injection is performed with a borosilicate glass capillary (0.78/1 mm internal/external diameters) pulled to a fine point, attached by polyethylene tubing (0.4/0.8 mm internal/external diameters) to a syringe mounted in a microinjection pump. The needle is mounted on an extended arm of a stereotaxic frame swung to the outside (used to hold and manipulate the needle only). Tubing, syringe, and needle are all filled with water. One microliter air is taken up into the needle followed by 3 μL of the viral vector solution. The needle is loaded separately with this volume for each injection. Animals are anesthetized prior to surgery. Following an incision along the dorsal midline, the L4 and L5 DRG are exposed by removal of the lateral processes of the vertebrae. The epineurium lying over the DRG is opened, and the glass needle inserted into the ganglion, to a depth of 400 μm from the surface of the exposed ganglion. After a 3-minute delay to allow sealing of the tissue around the glass capillary tip, 1.1 μL virus solution was injected at a rate of 0.2 μL/minute. After a further delay of 2 minutes, the needle is removed. The L4 ganglion is injected first followed by the L5 ganglion. The muscles overlying the spinal cord are loosely sutured together with a 5-0 suture and the wound closed. Animals are allowed to recover at 37° C. and received postoperative analgesia.

AAV intrathecal injections in rats: Rats are first anesthetized and then placed vertically with their head fixed in a stereotaxic frame. An incision is made in the base of the neck to expose the groove in the nuchal crest. An incision is made (1-2 mm) in the cisternal membrane to a depth such that cerebrospinal fluid leaks out. A 4 cm 32 G intrathecal catheter is then slowly inserted in the direction of the lumbar spinal cord and skin is closed by suture around the catheter. The rats are then allowed to recover. Rats are then anesthetized and the vector (6 μL) is administered. The catheter is flushed with 6 μL of PBS and then removed and rats allowed to recover.

Effects of administration: This SNI model is produced by the sectioning of the common peroneal and the sural nerves and isolating the tibial branch of the rat. The up-down method of Chaplan & Yaksh is used to determine mechanical thresholds before the injection of the AAV.hSYN-α7-nAChR/GlyRα1 into the spinal cord, DRG, or intrathecal space. Three weeks after unilateral vector injection, animals are tested again to verify that their mechanical withdrawal thresholds do not change. Motor coordination is also tested before and after injection, using an accelerating rotarod (Stoelting, USA) at a maximum speed of 33 rpm. The duration that the rat spends on the rotarod is recorded, with a cut-off at 300 sec. Each rat goes through three training trials and is tested two hours later.

Subsequently, half of the rats in each chimera cohort are administered a single IP injection of AZD-0328 or Facinicline and mechanical thresholds tested using the up-down method at 1, 2, 5, 7, and 13 days post IP injection. On the third day, when the thresholds has returned to post-injury baseline, AZD-0328 is again injected IP and again a recovery to non-injury baseline thresholds is observed. These animals are followed for 48 hours. Animals are then perfused for histology.

### Example 9: Treatment of a Patient Suffering from Chronic Pain

In a non-limiting example, a patient suffering from chronic radicular pain is treated using the compositions and methods disclosed herein. The patient is treated on Day One with 10¹³ vector genomes of AAV.hSYN operably linked to a polynucleotide encoding any one of the engineered receptors disclosed herein in a volume of 1.0 mL delivered directly into one or more dorsal root ganglia (i.e., intraganglionic convection-enhanced delivery into lumbar, cervical, or thoracic DRGs). In this example, the AAV vector encodes any one of the engineered receptors disclosed herein under the control of the human Synapsin-1 (SYN1) promoter for selective neuronal expression. Two weeks post-injection, the patient returns to the clinic for a prescription for AZD-0328 or another non-native ligand. The patient self-administers 0.1 mg/kg AZD-0328 or another non-native ligand orally as needed (i.e., during a pain episode).

### Example 10. Treatment of a Patient Suffering from Chronic Pain

In a non-limiting example, a patient suffering from chronic craniofacial pain (e.g. trigeminal neuralgia or termporomandibular joint dysfunction) is treated using the compositions and methods disclosed herein. The patient is treated on Day One with 10¹³ vector genomes of AAV.hSYN operably linked to a polynucleotide encoding any one of the engineered receptors disclosed herein in a volume of 0.150 mL delivered directly into the trigeminal ganglion (i.e., intraganglionic convection enhanced delivery). In this example, the AAV vector encodes any one of the engineered receptors disclosed herein under the control of the human Synapsin-1 (SYN1) promoter for selective neuronal expression. Two weeks post-injection, the patient returns to the clinic for a prescription for AZD-0328 or another non-native ligand. The patient self-administers 0.1 mg/kg AZD-0328 or another non-native ligand orally as needed (i.e., during a pain episode).

### Example 11. Treatment of a Patient Suffering from Obesity

In a non-limiting example, a patient suffering from obesity is treated using the compositions and methods disclosed herein. The patient is treated on Day One with 10¹³ vector genomes of AAV. Ghrelin operably linked to a polynucleotide encoding any one of the engineered receptors disclosed herein in a volume of 1.0 mL delivered directly into the gastric branch of the vagus nerve (i.e., intraneural). In this example, the AAV vector encodes the engineered receptor under the control of the human Ghrelin promoter for selective neuronal expression. Two weeks post-injection, the patient returns to the clinic for a prescription for AZD-0328 or another non-native ligand. The patient self-administers 0.1 mg/kg AZD-0328 or another non-native ligand orally daily for excess weight loss (i.e. for apetitite suppression).

### Example 12. Treatment of a Patient Suffering from Obesity

In a non-limiting example, a patient suffering from obesity is treated using the compositions and methods disclosed herein. The patient is treated on Day One with 10¹³ vector genomes of AAV-TRPV1 operably linked to a polynucleotide encoding any one of the engineered receptors disclosed herein in a volume of 1.0 mL delivered directly into the dorsal root ganglia innervating the pancreas (i.e., intragangionic). In this example, the AAV vector encodes the engineered receptor under the control of the human TRPV1 promoter for selective neuronal expression in nociceptors. Two weeks post-injection, the patient returns to the clinic for a prescription for AZD-0328 or another non-native ligand. The patient self-administers 0.1 mg/kg AZD-0328 or another non-native ligand orally daily for excess weight loss.

### Example 13. Treatment of a Patient Suffering from Obesity

In a non-limiting example, a patient suffering from obesity is treated using the compositions and methods disclosed herein. The patient is treated on Day One with 10¹³ vector genomes of AAV-SIM1 operably linked to a polynucleotide encoding any one of the engineered receptors disclosed herein in a volume of 1.0 mL delivered directly into the paraventricular nucleus (PVH) in the hypothalamus (i.e., intracranial, convection enhanced delivery). In this example, the AAV vector encodes the engineered channel under the control of the human Single-Minded Family BHLH Transcription Factor 1 (SIM1) promoter for selective neuronal expression in pro-opiomelanocortin (POMC) neurons and ultimately stimulation of the anorexigenic pathway. Two weeks post-injection, the patient returns to the clinic for a prescription for AZD-0328 or another non-native ligand. The patient self-administers 0.15 mg/kg AZD-0328 or another non-native ligand orally daily for excess weight loss (i.e. for apetitite suppression).

### Example 14. Treatment of a Patient Suffering from PTSD

In a non-limiting example, a patient suffering from post-traumatic stress disorder (PTSD) is treated using the compositions and methods disclosed herein. The patient is treated on Day One with 10¹³ vector genomes of AAV-hSYN1 operably linked to a polynucleotide encoding any one of the engineered receptors disclosed herein in a volume of 1.0 mL delivered directly into the C6 stellate ganglion, (i.e., intraganglionic). In this example, the AAV vector encodes the engineered receptor under the control of the human Synapsin-1 (hSYN1) promoter for selective neuronal expression. Two weeks post-injection, the patient returns to the clinic for a prescription for AZD-0328 or another non-native ligand. The patient self-administers 0.15 mg/kg AZD-0328 or another non-native ligand orally daily for PTSD symptoms (i.e. for anxiety).

### Example 15. Treatment of a Patient Suffering from Depression

In a non-limiting example, a patient suffering from treatment-resistant depression (TRD) is treated using the compositions and methods disclosed herein. The patient is treated on Day One with 10¹³ vector genomes of AAV-hSYN1 operably linked to a polynucleotide encoding any one of the engineered receptors disclosed herein in a volume of 1.0 mL delivered directly into the vagus nerve, (i.e., intraneural). In this example, the AAV vector encodes the engineered receptor under the control of the human Synapsin-1 (hSYN1) promoter for selective neuronal expression. Two weeks post-injection, the patient returns to the clinic for a prescription for AZD-0328 or another non-native ligand. The patient self-administers 0.1 mg/kg AZD-0328 or another non-native ligand orally daily for depression symptoms.

### Example 16. Treatment of a Patient Suffering from GERD

In a non-limiting example, a patient suffering from gastroesophageal reflux disease (GERD) is treated using the compositions and methods disclosed herein. The patient is treated on Day One with 10¹³ vector genomes of AAV-hSYN1 operably linked to a polynucleotide encoding any one of the engineered receptors disclosed herein or AAV-CAG operably linked to a polynucleotide encoding any one of the engineered receptors disclosed herein in a volume of 1.0 mL delivered directly into the lower esophageal sphincter (LES) vagus nerve and myenteric plexus (i.e., intraneural) or smooth muscle (intramuscular), respectively. In this example, the AAV vector encodes the engineered receptor under the control of the human Synapsin-1 (hSYN1) promoter for selective neuronal expression or the CAG promoter for expression in LES myocytes. Two weeks post-injection, the patient returns to the clinic for a prescription for AZD-0328 or another non-native ligand. The patient self-administers 0.15 mg/kg AD-0328 or another non-native ligand orally daily for symptoms of GERD (i.e. acid reflux).

### Example 17. Treatment of a Patient Suffering from Epilepsy

In a non-limiting example, a patient suffering from seizures associated with epilepsy is treated using the compositions and methods disclosed herein. The patient is treated on Day One with 10¹³ vector genomes of AAV-CamKIIα operably linked to a polynucleotide encoding any one of the engineered receptors disclosed herein in a volume of 1.0 mL delivered directly into a pre-determined seizure focus such as the motor cortex (i.e., intracranial). In this example, the AAV vector encodes the engineered receptor under the control of the human Calcium/calmodulin-dependent protein kinase II α (CamKIIα) promoter for selective neuronal expression in excitatory neurons. Two weeks post-injection, the patient returns to the clinic for a prescription for AZD-0328. The patient self-administers 0.1 mg/kg AZD-0328 orally daily for epileptic symptoms (i.e. seizures).

### Example 18. Treatment of a Patient Suffering from a Movement Disorder

In a non-limiting example, a patient suffering from a movement disorder (e.g. Parkinsonian tremor) is treated using the compositions and methods disclosed herein. The patient is treated on Day One with 10¹³ vector genomes of AAV-CamKIIα operably linked to a polynucleotide encoding any one of the engineered receptors disclosed herein in a volume of 1.0 mL delivered directly into the subthalamic nucleus (i.e., intracranial STN). In this example, the AAV vector encodes the engineered receptor under the control of the human Calcium/calmodulin-dependent protein kinase II α (CamKIIα) promoter for selective neuronal expression in excitatory neurons. Two weeks post-injection, the patient returns to the clinic for a prescription for AZD-0328. The patient self-administers 0.1 mg/kg AZD-0328 orally daily for movement disorder symptoms (i.e. tremor).

### FURTHER NUMBERED EMBODIMENTS

Further embodiments of the instant invention are provided in the numbered embodiments below:
Embodiment 1. An engineered receptor, comprising:
   a ligand binding domain derived from the human α7 nicotinic acetylcholine receptor (α7-nAChR) and comprising a Cys-loop domain from the human Glycine receptor al subunit; and
   an ion pore domain derived from the human Glycine receptor α1 subunit, wherein the ligand binding domain comprises: (i) two amino acid substitutions at a pair of amino acids residues selected from the group consisting of L131 and S172, Y115 and S170, and Y115 and L131; or (ii) an amino acid substitution of L131E, wherein the amino acid residues correspond to the amino acid residues of α7-nAChR, wherein the engineered receptor is a chimeric ligand gated ion channel (LGIC) receptor.
Embodiment 1.1 The engineered receptor of embodiment 1, wherein the engineered receptor comprises an amino acid sequence of SEQ ID NO: 33, wherein the amino acid sequence further comprises the two amino acid substitutions at a pair of amino acids residues selected from the group consisting of L131 and S172, Y115 and S170, and Y115 and L131; or the amino acid substitution of L131E, wherein the amino acid residues correspond to the amino acid residues of α7-nAChR.
Embodiment 2. The engineered receptor of embodiment 1 or 1.1, wherein the ligand binding domain comprises two amino acid substitutions at a pair of amino acids residues selected from the group consisting of L131 and S172, Y115 and S170, and Y115 and L131.
Embodiment 3. The engineered receptor of any one of embodiments 1, 1.1, or 2, wherein the ligand binding domain comprises a pair of amino acid substitutions selected from the group consisting of L131S and S172D, L131T and S172D, L131D and S172D, Y115D and S170T, Y115D and L131Q, and Y115D and L131E.
Embodiment 3.1 The engineered receptor of any one of embodiments 1-3, wherein the engineered receptor comprises an amino acid sequence of SEQ ID NO: 33, wherein the amino acid sequence further comprises a pair of amino acid substitutions selected from the group consisting of L131S and S172D, L131T and S172D, L131D and S172D, Y115D and S170T, Y115D and L131Q, and Y115D and L131E.
Embodiment 4. The engineered receptor of embodiment 1 or 1.1, wherein the ligand binding domain comprises an amino acid substitution of L131E.
Embodiment 4.1 The engineered receptor of any one of claims 1-4, wherein the engineered receptor comprises an amino acid sequence of SEQ ID NO: 33, wherein the amino acid sequence further comprises an amino acid substitution of L131E.
Embodiment 5. The engineered receptor of any one of embodiments 1-4.1, wherein the Cys-loop domain comprises amino acids 166-172 of SEQ ID NO: 2.
Embodiment 6. The engineered receptor of any one of embodiments 1-4.1, wherein the Cys-loop domain comprises amino acids 166-180 of SEQ ID NO: 2.
Embodiment 7. The engineered receptor of any one of embodiments 1-6, wherein the receptor comprises a β1-2 loop domain from the human Glycine receptor α1 subunit.
Embodiment 8. The engineered receptor of embodiment 7, wherein the β1-2 loop domain comprises amino acids 81-84 of SEQ ID NO:2.
Embodiment 8.1: The engineered receptor of any one of embodiments 1-8, wherein the engineered receptor comprises an amino acid sequence selected from the group consisting of SEQ ID Nos. 58-63.
Embodiment 9. The engineered receptor of any one of embodiments 1-8, wherein the potency of the engineered receptor to acetylcholine is lower than the potency of the human α7 nicotinic acetylcholine receptor (α7-nAChR) to acetylcholine.
Embodiment 10. The engineered receptor of embodiment 9, wherein the potency of the engineered receptor to acetylcholine is at least 2-fold lower than the potency of the human α7 nicotinic acetylcholine receptor (α7-nAChR) to acetylcholine.
Embodiment 11. The engineered receptor of any one of embodiments 1-10, wherein the potency of the engineered receptor to a non-native ligand is about the same as the potency of the human α7 nicotinic acetylcholine receptor (α7-nAChR) to the non-native ligand.
Embodiment 12. The engineered receptor of any one of embodiments 1-11, wherein the potency of the engineered receptor to a non-native ligand is higher than the potency of the human α7 nicotinic acetylcholine receptor (α7-nAChR) to the non-native ligand.
Embodiment 13. The engineered receptor of embodiment 12, wherein the potency of the engineered receptor to the non-native ligand is at least 2-fold higher than the potency of the human α7 nicotinic acetylcholine receptor (α7-nAChR) to the non-native ligand.
Embodiment 14. The engineered receptor of any one of embodiments 9-13, wherein determining the potency comprises determining the EC50.
Embodiment 15. The engineered receptor of any one of embodiments 1-14, wherein the efficacy of the engineered receptor in the presence of a non-native ligand is higher than the efficacy the human α7 nicotinic acetylcholine receptor (α7-nAChR) in presence of the non-native ligand.
Embodiment 16. The engineered receptor of any one of embodiments 1-15, wherein the efficacy of the engineered receptor in the presence of a non-native ligand is at least 2-fold higher than the efficacy the human α7 nicotinic acetylcholine receptor (α7-nAChR) in presence of the non-native ligand.
Embodiment 17. The engineered receptor of any one of embodiments 15-16, wherein determining the efficacy comprises determining the amount of current passed through the engineered receptor in vitro in the presence of the non-native ligand.
Embodiment 18. The engineered receptor of any one of embodiments 11-17, wherein the non-native ligand is selected from the group consisting of AZD-0328, TC-6987, ABT-126, APN-1125, TC-5619, and Facinicline/RG3487.
Embodiment 19. The engineered receptor of embodiment 18, wherein the non-native ligand is selected from the group consisting of ABT-126, RG3487, and APN-1125.
Embodiment 20. The engineered receptor of embodiment 18, wherein the non-native ligand is TC-5619.
Embodiment 21. A polynucleotide, comprising a nucleic acid encoding the engineered receptor of any one of embodiments 1-20.
Embodiment 22. The polynucleotide of embodiment 21, wherein the polynucleotide comprises a promoter operably linked to the nucleic acid encoding the engineered receptor.
Embodiment 23. The polynucleotide of embodiment 22, wherein the promoter is a regulatable promoter.
Embodiment 24. The polynucleotide of embodiment 23, wherein the regulatable promoter is active in an excitable cell.
Embodiment 25. The polynucleotide of embodiment 24, wherein the excitable cell is a neuron or a myocyte.
Embodiment 26. The polynucleotide of embodiment 25, wherein the excitable cell is a neuron.
Embodiment 27. A vector comprising the polynucleotide of any one of embodiments 21-26.
Embodiment 28. The vector of embodiment 27, wherein the vector is a plasmid, or a viral vector.
Embodiment 29. The vector of embodiment 28, wherein the vector is a viral vector selected from the group consisting of an adenoviral vector, a retroviral vector, an adeno-associated viral (AAV) vector, and a herpes simplex-1 viral vector (HSV-1).
Embodiment 30. The vector of embodiment 29, wherein the viral vector is an AVV vector, and wherein the AAV vector is AAVS or a variant thereof, AAV6 or a variant thereof or AAV9 or a variant thereof.
Embodiment 31. A composition comprising the engineered receptor of any one of embodiments 1-20, the polynucleotide of any one of embodiments 21-26, or the vector of any one of embodiments 27-30.
Embodiment 32. A pharmaceutical composition comprising the engineered receptor of any one of embodiments 1-20, the polynucleotide of any one of embodiments 21-26, or the vector of any one of embodiments 27-30; and a pharmaceutically acceptable carrier.
Embodiment 33. A method of producing an engineered receptor in a neuron, comprising contacting the neuron with the polynucleotide of any one of embodiments 21-26, the vector of any one of embodiments 27-30, the composition of embodiment 31, or the pharmaceutical composition of embodiment 32.
Embodiment 34. The method of embodiment 33 or the polynucleotide of embodiment 26, wherein the neuron is a neuron of the peripheral nervous system.
Embodiment 35. The method of embodiment 33 or 34, or the polynucleotide of embodiment 26, wherein the neuron is a neuron of the central nervous system.
Embodiment 36. The method of any one of embodiments 33-35 or the polynucleotide of embodiment 26, wherein the neuron is a nociceptive neuron.
Embodiment 37. The method of any one of embodiments 33-36 or the polynucleotide of embodiment 26, wherein the neuron is a non-nociceptive neuron.
Embodiment 38. The method of any one of embodiments 33-37 or the polynucleotide of embodiment 26, wherein the neuron is a dorsal root ganglion (DRG) neuron, a trigeminal ganglion (TG) neuron, a motor neuron, an excitatory neuron, an inhibitory neuron, or a sensory neuron.
Embodiment 39. The method of any one of embodiments 33-38 or the polynucleotide of embodiment 26, wherein the neuron is an Aδ afferent fiber, a C fiber or an Aβ afferent fiber.
Embodiment 40. The method of embodiment 39 or the polynucleotide of embodiment 26, wherein the neuron is Aβ afferent fiber.
Embodiment 41. The method of embodiment 40 or the polynucleotide of embodiment 26, wherein Aβ afferent fiber is an injured Aβ afferent fiber.
Embodiment 42. The method of embodiment 40 or the polynucleotide of embodiment 26, wherein Aβ afferent fiber is an uninjured Aβ afferent fiber.
Embodiment 43. The method of any one of embodiments 33-42 or the polynucleotide of embodiment 26, wherein the neuron expresses neurofilament 200 (NF200), piezo 2, and TLR-5.
Embodiment 44. The method of any one of embodiments 33-43 or the polynucleotide of embodiment 26, wherein the neuron does not express TrpV1, prostatic acid phosphatase, NaV1.1.
Embodiment 45. The method of any one of embodiments 33-44, wherein the contacting step is performed in vitro, ex vivo, or in vivo.
Embodiment 46. The method of embodiment 45, wherein the contacting step is performed in vivo in a subject.
Embodiment 47. The method of embodiment 46, wherein the contacting step comprises administering the polynucleotide, the vector, the composition, or the pharmaceutical composition to the subject.
Embodiment 48. The method of embodiment 45, wherein the contacting step is performed in vitro or ex vivo.
Embodiment 49. The method of embodiment 48, wherein the contacting step comprises lipofection, nanoparticle delivery, particle bombardment, electroporation, sonication, or microinjection.
Embodiment 50. The method of any one of embodiments 33-49, wherein the engineered receptor is capable of localizing to the cell surface of the neuron.
Embodiment 51. A method of inhibiting the activity of a neuron, comprising (a) contacting the neuron with the engineered receptor of any one of embodiments 1-20, the polynucleotide of any one of embodiments 21-26, the vector of any one of embodiments 27-30, the composition of embodiment 31, or the pharmaceutical composition of embodiment 32, and (b) contacting the neuron with a non-native ligand of the engineered receptor.
Embodiment 51.1 The method of embodiment 51, wherein the neuron is a neuron of the peripheral nervous system.
Embodiment 51.2 The method of embodiment 51, wherein the neuron is a neuron of the central nervous system.
Embodiment 52. The method of any of the embodiments 51-51.2, wherein the neuron is a nociceptive neuron.
Embodiment 53. The method of any of the embodiments 51-51.2, wherein the neuron is a non-nociceptive neuron.
Embodiment 54. The method of any one of embodiments 51-53, wherein the neuron is a dorsal root ganglion (DRG) neuron, a trigeminal ganglion (TG) neuron, a motor neuron, an excitatory neuron, an inhibitory neuron, or a sensory neuron.
Embodiment 55. The method of any one of embodiments 51-54, wherein the neuron is an Aδ afferent fiber, a C fiber or an Aβ afferent fiber.
Embodiment 56. The method of embodiment 55, wherein the neuron is Aβ afferent fiber.
Embodiment 57. The method of embodiment 56, wherein Aβ afferent fiber is an injured Aβ afferent fiber.
Embodiment 58. The method of embodiment 56, wherein Aβ afferent fiber is an uninjured Aβ afferent fiber.
Embodiment 59. The method of any one of embodiments 51-58, wherein the neuron expresses neurofilament 200 (NF200), piezo 2, and TLR-5.
Embodiment 60. The method of any one of embodiments 51-59, wherein the neuron does not express TrpV1, prostatic acid phosphatase, NaV1.1.
Embodiment 61. The method of any one of embodiments 51-60, wherein the contacting step (a) is performed in vitro, ex vivo, or in vivo.
Embodiment 62. The method of any one of embodiments 51-61, wherein the contacting step (b) is performed in vitro, ex vivo, or in vivo.
Embodiment 63. The method of any one of embodiments 51-62, wherein the contacting steps (a) and/or (b) are performed in vivo in a subject.
Embodiment 64. The method of embodiment 63, wherein the contacting step (a) comprises administering the engineered receptor, the polynucleotide, the vector, or the pharmaceutical composition to the subject; and/or the contacting step (b) comprises administering the non-native ligand to the subject.
Embodiment 65. The method of any one of embodiments 51-64, wherein the contacting step (a) and/or (b) comprises lipofection, nanoparticle delivery, particle bombardment, electroporation, sonication, or microinjection.
Embodiment 66. The method of any one of embodiments 51-65, wherein the engineered receptor is capable of localizing to the cell surface of the neuron.
Embodiment 67. A method of treating and/or delaying the onset of a neurological disorder in a subject, in need thereof, comprising:
   administering to the subject, a therapeutically effective amount of the engineered receptor of any one of embodiments 1-20, the polynucleotide of any one of embodiments 21-26, the vector of any one of embodiments 27-30, the composition of embodiment 31, or the pharmaceutical composition of embodiment 32, and
   administering to the subject a non-native ligand of the engineered receptor.
Embodiment 68. The method of embodiment 67, wherein the subject is administered the non-native ligand after step (a).
Embodiment 69. The method of embodiment 67, wherein the subject is administered the non-native ligand concurrently with step (a).
Embodiment 70. The method of any one of embodiments 67-69, wherein the neurological disorder is a seizure disorder, a movement disorder, an eating disorder, a spinal cord injury, neurogenic bladder, allodynia, a spasticity disorder, pruritus, Alzheimer's disease, Parkinson's disease, post-traumatic stress disorder (PTSD), gastroesophageal reflux disease (GERD), addiction, anxiety, depression, memory loss, dementia, sleep apnea, stroke, narcolepsy, urinary incontinence, essential tremor, trigeminal neuralgia, burning mouth syndrome, or atrial fibrillation.
Embodiment 71. The method of embodiment 70, wherein the neurological disorder is allodynia.
Embodiment 72. The method of any one of embodiments 67-71, wherein the non-native ligand is selected from the group consisting of AZD-0328, ABT-126, TC6987, APN-1125, TC-5619, and Facinicline/RG3487.
Embodiment 73. The method of any one of embodiments 67-72, wherein the non-native ligand is administered orally, subcutaneously, topically, or intravenously.
Embodiment 74. The method of embodiment 73, wherein the non-native ligand is administered orally.
Embodiment 75. The method of any one of embodiments 67-74, wherein the engineered receptor, the polynucleotide, the vector, the composition, or the pharmaceutical composition is administered subcutaneously, orally, intrathecally, topically, intravenously, intraganglioncally, intraneurally, intracranially, intraspinally, or to the cisterna magna.
Embodiment 76. The method of any one of embodiments 67-75, wherein the engineered receptor, the polynucleotide, the vector, the composition, or the pharmaceutical composition is administered by transforaminal injection or intrathecally.
Embodiment 77. The method of any one of embodiments 67-76, wherein the subject suffers from trigeminal neuralgia, and wherein the engineered receptor, the polynucleotide, the vector, the composition, or the pharmaceutical composition is administered to the trigeminal ganglion (TG) of the subject.
Embodiment 78. The method of any one of embodiments 67-76, wherein the subject suffers from neuropathic pain, and wherein the engineered receptor, the polynucleotide, the vector, the composition, or the pharmaceutical composition is administered to the dorsal root ganglion (DRG) of the subject.
Embodiment 79. The method of any one of embodiments 67-78, wherein the subject is a human.
Embodiment 80. The method of any one of embodiments 67-79, wherein the therapeutically effectively amount diminishes the severity of a sign and/or or a symptom of the neurological disorder.
Embodiment 81. The method of any one of embodiments 67-80, wherein the therapeutically effectively amount delays the onset of a sign and/or or a symptom of the neurological disorder.
Embodiment 82. The method of any one of embodiments 67-81, wherein the therapeutically effectively amount eliminates a sign and/or or a symptom of the neurological disorder.
Embodiment 83. The method of any one of embodiments 80-82, wherein the sign of the neurological disorder is nerve damage, nerve atrophy, and/or seizure.
Embodiment 84. The method of embodiment 83, wherein the nerve damage is peripheral nerve damage.
Embodiment 85. The method of any one of embodiments 80-84, wherein the symptom of the neurological disorder is pain.
Embodiment 86. A method of treating and/or delaying the onset of pain in a subject, in need thereof, comprising:
   administering to the subject, a therapeutically effective amount of the engineered receptor of any one of embodiments 1-20, the polynucleotide of any one of embodiments 21-26, the vector of any one of embodiments 27-30, the composition of embodiment 31, or the pharmaceutical composition of embodiment 32, and
   administering to the subject a non-native ligand of the engineered receptor.
Embodiment 87. The method of embodiment 86, wherein the subject is administered the non-native ligand after step (a).
Embodiment 88. The method of embodiment 86, wherein the subject is administered the non-native ligand concurrently with step (a).
Embodiment 89. The method of any one of embodiments 86-88, wherein the non-native ligand is selected from the group consisting of AZD-0328, ABT-126, TC6987, APN-1125, TC-5619, and Facinicline/RG3487.
Embodiment 90. The method of any one of embodiments 86-89, wherein the non-native ligand is administered orally, subcutaneously, topically, or intravenously.
Embodiment 91. The method of embodiment 90, wherein the non-native ligand is administered orally.
Embodiment 92. The method of any one of embodiments 86-91, wherein the engineered receptor, the polynucleotide, the vector, the composition, or the pharmaceutical composition is administered subcutaneously, orally, intrathecally, topically, intravenously, intraganglioncally, intraneurally, intracranially, intraspinally, or to the cisterna magna.
Embodiment 93. The method of any one of embodiments 86-92, wherein the engineered receptor, the polynucleotide, the vector, the composition, or the pharmaceutical composition is administered by transforaminal injection or intrathecally.
Embodiment 94. The method of any one of embodiments 86-93, wherein the subject suffers from trigeminal neuralgia, and wherein the engineered receptor, the polynucleotide, the vector, the composition, or the pharmaceutical composition is administered to the trigeminal ganglion (TG) of the subject.
Embodiment 95. The method of any one of embodiments 86-94, wherein the subject suffers from neuropathic pain, and wherein the engineered receptor, the polynucleotide, the vector, the composition, or the pharmaceutical composition is administered to the dorsal root ganglion (DRG) of the subject.
Embodiment 96. The method of any one of embodiments 86-95, wherein the subject is a human.
Embodiment 97. The method of any one of embodiments 85-96, wherein the pain is neuropathic pain.
Embodiment 98. The method of any one of embodiments 85-97, wherein the pain is associated with, caused by, or resulting from chemotherapy.
Embodiment 99. The method of any one of embodiments 85-98, wherein the pain is associated with, caused by, or resulting from trauma.
Embodiment 100. The method of any of embodiments 85-99, wherein the subject suffers from allodynia.
Embodiment 101. The method of any one of embodiments 85-100, wherein the pain manifests after a medical procedure.
Embodiment 102. The method of any one of embodiments 85-101, wherein the pain is associated with, is caused by, or resulting from childbirth or Caesarean section.
Embodiment 103. The method of any one of embodiments 85-102, wherein the pain is associated with, is caused by, or resulting from migraine.
Embodiment 104. The method of any one of embodiments 85-103, wherein the therapeutically effectively amount diminishes pain in the subject transiently, diminishes pain in the subject permanently, prevents the onset of pain in the subject, and/or eliminates pain in the subject.
Embodiment 105. The method of any one of embodiments 85-104, wherein steps (a) and (b) are performed before the manifestation of pain in the subject.

The preceding merely illustrates the principles of the disclosure. It will be appreciated that those skilled in the art will be able to devise various arrangements which, although not explicitly described or shown herein, embody the principles of the disclosure and are included within its spirit and scope. Furthermore, all examples and conditional language recited herein are principally intended to aid the reader in understanding the principles of the disclosure and the concepts contributed by the inventors to furthering the art, and are to be construed as being without limitation to such specifically recited examples and conditions. Moreover, all statements herein reciting principles, aspects, and embodiments of the disclosure as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. Rather, the scope and spirit of the present disclosure is embodied by the appended claims.

What is claimed is:

1. An engineered receptor, comprising:
a. a ligand binding domain derived from the human α7 nicotinic acetylcholine receptor (α7-nAChR) and comprising a Cys-loop domain from the human Glycine receptor α1 subunit; and
b. an ion pore domain derived from the human Glycine receptor α1 subunit,
wherein the ligand binding domain comprises: (i) two amino acid substitutions at a pair of amino acids residues selected from the group consisting of L131 and S172, Y115 and S170, and Y115 and L131; or (ii) an amino acid substitution of L131E, wherein the amino acid residues correspond to the amino acid residues of α7-nAChR,
wherein the engineered receptor is a chimeric ligand gated ion channel (LGIC) receptor.

1.1. The engineered receptor of claim 1, wherein the engineered receptor comprises an amino acid sequence of SEQ ID NO: 33, wherein the amino acid sequence further comprises the two amino acid substitutions at a pair of amino acids residues selected from the group consisting of L131 and S172, Y115 and S170, and Y115 and L131; or the amino acid substitution of L131E, wherein the amino acid residues correspond to the amino acid residues of α7-nAChR.

2. The engineered receptor of claim 1 or 1.1, wherein the ligand binding domain comprises two amino acid substitutions at a pair of amino acids residues selected from the group consisting of L131 and S172, Y115 and S170, and Y115 and L131.

3. The engineered receptor of any one of claim 1, 1.1 or 2, wherein the ligand binding domain comprises a pair of amino acid substitutions selected from the group consisting of L131S and S172D, L131T and S172D, L131D and S172D, Y115D and S170T, Y115D and L131Q, and Y115D and L131E.

3.1. The engineered receptor of any one of claims 1-3, wherein the engineered receptor comprises an amino acid sequence of SEQ ID NO: 33, wherein the amino acid sequence further comprises a pair of amino acid substitutions selected from the group consisting of L131S and S172D, L131T and S172D, L131D and S172D, Y115D and S170T, Y115D and L131Q, and Y115D and L131E.

4. The engineered receptor of claim 1 or 1.1, wherein the ligand binding domain comprises an amino acid substitution of L131E.

4.1. The engineered receptor of any one of claims 1-4, wherein the engineered receptor comprises an amino acid sequence of SEQ ID NO: 33, wherein the amino acid sequence further comprises an amino acid substitution of L131E.

5. The engineered receptor of any one of claims 1-4.1, wherein the Cys-loop domain comprises amino acids 166-172 of SEQ ID NO: 2.

6. The engineered receptor of any one of claims 1-4.1, wherein the Cys-loop domain comprises amino acids 166-180 of SEQ ID NO: 2.

7. The engineered receptor of any one of claims 1-6, wherein the receptor comprises a β1-2 loop domain from the human Glycine receptor α1 subunit.

8. The engineered receptor of claim 7, wherein the β1-2 loop domain comprises amino acids 81-84 of SEQ ID NO:2.

8.1. The engineered receptor of any one of claims 1-8, wherein the engineered receptor comprises an amino acid sequence selected from the group consisting of SEQ ID Nos. 58-63.

9. The engineered receptor of any one of claims 1-8, wherein the potency of the engineered receptor to acetylcholine is lower than the potency of the human α7 nicotinic acetylcholine receptor (α7-nAChR) to acetylcholine.

10. The engineered receptor of claim 9, wherein the potency of the engineered receptor to acetylcholine is at least 2-fold lower than the potency of the human α7 nicotinic acetylcholine receptor (α7-nAChR) to acetylcholine.

11. The engineered receptor of any one of claims 1-10, wherein the potency of the engineered receptor to a non-native ligand is about the same as the potency of the human α7 nicotinic acetylcholine receptor (α7-nAChR) to the non-native ligand.

12. The engineered receptor of any one of claims 1-11, wherein the potency of the engineered receptor to a non-native ligand is higher than the potency of the human α7 nicotinic acetylcholine receptor (α7-nAChR) to the non-native ligand.

13. The engineered receptor of claim 12, wherein the potency of the engineered receptor to the non-native ligand is at least 2-fold higher than the potency of the human α7 nicotinic acetylcholine receptor (α7-nAChR) to the non-native ligand.

14. The engineered receptor of any one of claims 9-13, wherein determining the potency comprises determining the EC50.

15. The engineered receptor of any one of claims 1-14, wherein the efficacy of the engineered receptor in the presence of a non-native ligand is higher than the efficacy the human α7 nicotinic acetylcholine receptor (α7-nAChR) in presence of the non-native ligand.

16. The engineered receptor of any one of claims 1-15, wherein the efficacy of the engineered receptor in the presence of a non-native ligand is at least 2-fold higher than the efficacy the human α7 nicotinic acetylcholine receptor (α7-nAChR) in presence of the non-native ligand.

17. The engineered receptor of any one of claims 15-16, wherein determining the efficacy comprises determining the amount of current passed through the engineered receptor in vitro in the presence of the non-native ligand.

18. The engineered receptor of any one of claims 11-17, wherein the non-native ligand is selected from the group consisting of AZD-0328, TC-6987, ABT-126, APN-1125, TC-5619, and Facinicline/RG3487.

19. The engineered receptor of claim 18, wherein the non-native ligand is selected from the group consisting of ABT-126, RG3487, and APN-1125.

20. The engineered receptor of claim 18, wherein the non-native ligand is TC-5619.

21. A polynucleotide, comprising a nucleic acid encoding the engineered receptor of any one of claims 1-20.

22. The polynucleotide of claim 21, wherein the polynucleotide comprises a promoter operably linked to the nucleic acid encoding the engineered receptor.

23. The polynucleotide of claim 22, wherein the promoter is a regulatable promoter.

24. The polynucleotide of claim 23, wherein the regulatable promoter is active in an excitable cell.

25. The polynucleotide of claim 24, wherein the excitable cell is a neuron or a myocyte.

26. The polynucleotide of claim 25, wherein the excitable cell is a neuron.

27. A vector comprising the polynucleotide of any one of claims 21-26.

28. The vector of claim 27, wherein the vector is a plasmid, or a viral vector.

29. The vector of claim 28, wherein the vector is a viral vector selected from the group consisting of an adenoviral vector, a retroviral vector, an adeno-associated viral (AAV) vector, and a herpes simplex-1 viral vector (HSV-1).

30. The vector of claim 29, wherein the viral vector is an AVV vector, and wherein the AAV vector is AAVS or a variant thereof, AAV6 or a variant thereof or AAV9 or a variant thereof.

31. A composition comprising the engineered receptor of any one of claims 1-20, the polynucleotide of any one of claims 21-26, or the vector of any one of claims 27-30.

32. A pharmaceutical composition comprising the engineered receptor of any one of claims 1-20, the polynucleotide of any one of claims 21-26, or the vector of any one of claims 27-30; and a pharmaceutically acceptable carrier.

33. A method of producing an engineered receptor in a neuron, comprising contacting the neuron with the polynucleotide of any one of claims 21-26, the vector of any one of claims 27-30, the composition of claim 31, or the pharmaceutical composition of claim 32.

34. The method of claim 33 or the polynucleotide of claim 26, wherein the neuron is a neuron of the peripheral nervous system.

35. The method of claim 33 or 34, or the polynucleotide of claim 26, wherein the neuron is a neuron of the central nervous system.

36. The method of any one of claims 33-35 or the polynucleotide of claim 26, wherein the neuron is a nociceptive neuron.

37. The method of any one of claims 33-36 or the polynucleotide of claim 26, wherein the neuron is a non-nociceptive neuron.

38. The method of any one of claims 33-37 or the polynucleotide of claim 26, wherein the neuron is a dorsal root ganglion (DRG) neuron, a trigeminal ganglion (TG) neuron, a motor neuron, an excitatory neuron, an inhibitory neuron, or a sensory neuron.

39. The method of any one of claims 33-38 or the polynucleotide of claim 26, wherein the neuron is an Aδ afferent fiber, a C fiber or an Aβ afferent fiber.

40. The method of claim 39 or the polynucleotide of claim 26, wherein the neuron is Aβ afferent fiber.

41. The method of claim 40 or the polynucleotide of claim 26, wherein Aβ afferent fiber is an injured Aβ afferent fiber.

42. The method of claim 40 or the polynucleotide of claim 26, wherein Aβ afferent fiber is an uninjured Aβ afferent fiber.

43. The method of any one of claims 33-42 or the polynucleotide of claim 26, wherein the neuron expresses neurofilament 200 (NF200), piezo 2, and TLR-5.

44. The method of any one of claims 33-43 or the polynucleotide of claim 26, wherein the neuron does not express TrpV1, prostatic acid phosphatase, NaV1.1.

45. The method of any one of claims 33-44, wherein the contacting step is performed in vitro, ex vivo, or in vivo.

46. The method of claim 45, wherein the contacting step is performed in vivo in a subject.

47. The method of claim 46, wherein the contacting step comprises administering the polynucleotide, the vector, the composition, or the pharmaceutical composition to the subject.

48. The method of claim 45, wherein the contacting step is performed in vitro or ex vivo.

49. The method of claim 48, wherein the contacting step comprises lipofection, nanoparticle delivery, particle bombardment, electroporation, sonication, or microinjection.

50. The method of any one of claims 33-49, wherein the engineered receptor is capable of localizing to the cell surface of the neuron.

51. A method of inhibiting the activity of a neuron, comprising (a) contacting the neuron with the engineered receptor of any one of claims 1-20, the polynucleotide of any one of claims 21-26, the vector of any one of claims 27-30, the composition of claim 31, or the pharmaceutical composition of claim 32, and (b) contacting the neuron with a non-native ligand of the engineered receptor.

51.1. The method of claim 51, wherein the neuron is a neuron of the peripheral nervous system.

51.2. The method of claim 51, wherein the neuron is a neuron of the central nervous system.

52. The method of any of the claims 51-51.2, wherein the neuron is a nociceptive neuron.

53. The method of any of the claims 51-51.2, wherein the neuron is a non-nociceptive neuron.

54. The method of any one of claims 51-53, wherein the neuron is a dorsal root ganglion (DRG) neuron, a trigeminal ganglion (TG) neuron, a motor neuron, an excitatory neuron, an inhibitory neuron, or a sensory neuron.

55. The method of any one of claims 51-54, wherein the neuron is an Aδ afferent fiber, a C fiber or an Aβ afferent fiber.

56. The method of claim 55, wherein the neuron is Aβ afferent fiber.

57. The method of claim 56, wherein Aβ afferent fiber is an injured Aβ afferent fiber.

58. The method of claim 56, wherein Aβ afferent fiber is an uninjured Aβ afferent fiber.

59. The method of any one of claims 51-58, wherein the neuron expresses neurofilament 200 (NF200), piezo 2, and TLR-5.

60. The method of any one of claims 51-59, wherein the neuron does not express TrpV1, prostatic acid phosphatase, NaV1.1.

61. The method of any one of claims 51-60, wherein the contacting step (a) is performed in vitro, ex vivo, or in vivo.

62. The method of any one of claims 51-61, wherein the contacting step (b) is performed in vitro, ex vivo, or in vivo.

63. The method of any one of claims 51-62, wherein the contacting steps (a) and/or (b) are performed in vivo in a subject.

64. The method of claim 63, wherein the contacting step (a) comprises administering the engineered receptor, the polynucleotide, the vector, or the pharmaceutical composition to the subject; and/or the contacting step (b) comprises administering the non-native ligand to the subject.

65. The method of any one of claims 51-64, wherein the contacting step (a) and/or (b) comprises lipofection, nanoparticle delivery, particle bombardment, electroporation, sonication, or microinjection.

66. The method of any one of claims 51-65, wherein the engineered receptor is capable of localizing to the cell surface of the neuron.

67. A method of treating and/or delaying the onset of a neurological disorder in a subject, in need thereof, comprising:
a. administering to the subject, a therapeutically effective amount of the engineered receptor of any one of claims 1-20, the polynucleotide of any one of claims 21-26, the vector of any one of claims 27-30, the composition of claim 31, or the pharmaceutical composition of claim 32, and
b. administering to the subject a non-native ligand of the engineered receptor.

68. The method of claim 67, wherein the subject is administered the non-native ligand after step (a).

69. The method of claim 67, wherein the subject is administered the non-native ligand concurrently with step (a).

70. The method of any one of claims 67-69, wherein the neurological disorder is a seizure disorder, a movement disorder, an eating disorder, a spinal cord injury, neurogenic bladder, allodynia, a spasticity disorder, pruritus, Alzheimer's disease, Parkinson's disease, post-traumatic stress disorder (PTSD), gastroesophageal reflux disease (GERD), addiction, anxiety, depression, memory loss, dementia, sleep apnea, stroke, narcolepsy, urinary incontinence, essential tremor, trigeminal neuralgia, burning mouth syndrome, or atrial fibrillation.

71. The method of claim 70, wherein the neurological disorder is allodynia.

72. The method of any one of claims 67-71, wherein the non-native ligand is selected from the group consisting of AZD-0328, ABT-126, TC6987, APN-1125, TC-5619, and Facinicline/RG3487.

73. The method of any one of claims 67-72, wherein the non-native ligand is administered orally, subcutaneously, topically, or intravenously.

74. The method of claim 73, wherein the non-native ligand is administered orally.

75. The method of any one of claims 67-74, wherein the engineered receptor, the polynucleotide, the vector, the composition, or the pharmaceutical composition is administered subcutaneously, orally, intrathecally, topically, intravenously, intraganglioncally, intraneurally, intracranially, intraspinally, or to the cisterna magna.

76. The method of any one of claims 67-75, wherein the engineered receptor, the polynucleotide, the vector, the composition, or the pharmaceutical composition is administered by transforaminal injection or intrathecally.

77. The method of any one of claims 67-76, wherein the subject suffers from trigeminal neuralgia, and wherein the engineered receptor, the polynucleotide, the vector, the composition, or the pharmaceutical composition is administered to the trigeminal ganglion (TG) of the subject.

78. The method of any one of claims 67-76, wherein the subject suffers from neuropathic pain, and wherein the engineered receptor, the polynucleotide, the vector, the composition, or the pharmaceutical composition is administered to the dorsal root ganglion (DRG) of the subject.

79. The method of any one of claims 67-78, wherein the subject is a human.

80. The method of any one of claims 67-79, wherein the therapeutically effectively amount diminishes the severity of a sign and/or or a symptom of the neurological disorder.

81. The method of any one of claims 67-80, wherein the therapeutically effectively amount delays the onset of a sign and/or or a symptom of the neurological disorder.

82. The method of any one of claims 67-81, wherein the therapeutically effectively amount eliminates a sign and/or or a symptom of the neurological disorder.

83. The method of any one of claims 80-82, wherein the sign of the neurological disorder is nerve damage, nerve atrophy, and/or seizure.

84. The method of claim 83, wherein the nerve damage is peripheral nerve damage.

85. The method of any one of claims 80-84, wherein the symptom of the neurological disorder is pain.

86. A method of treating and/or delaying the onset of pain in a subject, in need thereof, comprising:
a. administering to the subject, a therapeutically effective amount of the engineered receptor of any one of claims 1-20, the polynucleotide of any one of claims 21-26, the vector of any one of claims 27-30, the composition of claim 31, or the pharmaceutical composition of claim 32, and
b. administering to the subject a non-native ligand of the engineered receptor.

87. The method of claim 86, wherein the subject is administered the non-native ligand after step (a).

88. The method of claim 86, wherein the subject is administered the non-native ligand concurrently with step (a).

89. The method of any one of claims 86-88, wherein the non-native ligand is selected from the group consisting of AZD-0328, ABT-126, TC6987, APN-1125, TC-5619, and Facinicline/RG3487.

90. The method of any one of claims 86-89, wherein the non-native ligand is administered orally, subcutaneously, topically, or intravenously.

91. The method of claim 90, wherein the non-native ligand is administered orally.

92. The method of any one of claims 86-91, wherein the engineered receptor, the polynucleotide, the vector, the composition, or the pharmaceutical composition is administered subcutaneously, orally, intrathecally, topically, intravenously, intraganglioncally, intraneurally, intracranially, intraspinally, or to the cisterna magna.

93. The method of any one of claims 86-92, wherein the engineered receptor, the polynucleotide, the vector, the composition, or the pharmaceutical composition is administered by transforaminal injection or intrathecally.

94. The method of any one of claims 86-93, wherein the subject suffers from trigeminal neuralgia, and wherein the engineered receptor, the polynucleotide, the vector, the composition, or the pharmaceutical composition is administered to the trigeminal ganglion (TG) of the subject.

95. The method of any one of claims 86-94, wherein the subject suffers from neuropathic pain, and wherein the engineered receptor, the polynucleotide, the vector, the composition, or the pharmaceutical composition is administered to the dorsal root ganglion (DRG) of the subject.

96. The method of any one of claims 86-95, wherein the subject is a human.

97. The method of any one of claims 85-96, wherein the pain is neuropathic pain.

98. The method of any one of claims 85-97, wherein the pain is associated with, caused by, or resulting from chemotherapy.

99. The method of any one of claims 85-98, wherein the pain is associated with, caused by, or resulting from trauma.

100. The method of any of claims 85-99, wherein the subject suffers from allodynia.

101. The method of any one of claims 85-100, wherein the pain manifests after a medical procedure.

102. The method of any one of claims 85-101, wherein the pain is associated with, is caused by, or resulting from childbirth or Caesarean section.

103. The method of any one of claims 85-102, wherein the pain is associated with, is caused by, or resulting from migraine.

104. The method of any one of claims 85-103, wherein the therapeutically effectively amount diminishes pain in the subject transiently, diminishes pain in the subject permanently, prevents the onset of pain in the subject, and/or eliminates pain in the subject.

105. The method of any one of claims 85-104, wherein steps (a) and (b) are performed before the manifestation of pain in the subject.
